SEQUENCE LISTING (102 sequences)

SEQ ID NO: 1 - DNA, 29 bp, Artificial Sequence
  Description of Artificial Sequence Synthetic oligonucleotide
aggggcaggctagctacaacgacgtcgtg

SEQ ID NO: 2 - DNA, 33 bp, Artificial Sequence
  Description of Artificial Sequence Synthetic oligonucleotide
tgaggggcaggctagctacaacgacgtcgtgac

SEQ ID NO: 3 - DNA, 33 bp, Artificial Sequence; identical to SEQ ID NO: 2
  Description of Artificial Sequence Synthetic oligonucleotide

SEQ ID NO: 4 - DNA, 31 bp, Artificial Sequence
  Description of Artificial Sequence Synthetic oligonucleotide
gaggggcaggctagctacaacgacgtcgtga

SEQ ID NO: 5 - DNA, 33 bp, Artificial Sequence; identical to SEQ ID NO: 2
  Description of Artificial Sequence Synthetic oligonucleotide

SEQ ID NO: 6 - DNA, 33 bp, Artificial Sequence
  Description of Artificial Sequence Synthetic oligonucleotide
tgaggggcaggctagctacaacgacgtcgcggg

SEQ ID NO: 7 - DNA, 25 bp, Artificial Sequence
  Description of Artificial Sequence Synthetic oligonucleotide
tgaggggcaggctagctacaacgat

SEQ ID NO: 8 - DNA, 10 bp, Artificial Sequence
  Description of Artificial Sequence Synthetic oligonucleotide
tgaggggcat

SEQ ID NO: 9 - DNA, 33 bp, Artificial Sequence; identical to SEQ ID NO: 2
  Description of Artificial Sequence Synthetic oligonucleotide

SEQ ID NO: 10 - DNA, 33 bp, Artificial Sequence; identical to SEQ ID NO: 2
  Description of Artificial Sequence Synthetic oligonucleotide

SEQ ID NO: 11 - DNA, 33 bp, Artificial Sequence; identical to SEQ ID NO: 2
  Description of Artificial Sequence Synthetic oligonucleotide

SEQ ID NO: 12 - DNA, 33 bp, Artificial Sequence; identical to SEQ ID NO: 2
  Description of Artificial Sequence Synthetic oligonucleotide

SEQ ID NO: 13 - DNA, 33 bp, Artificial Sequence; identical to SEQ ID NO: 2
  Description of Artificial Sequence Synthetic oligonucleotide

SEQ ID NO: 14 - DNA, 33 bp, Artificial Sequence
  Description of Artificial Sequence Synthetic oligonucleotide
tgaggggcaagcaacatcgatcggcgtcgtgac

SEQ ID NO: 15 - DNA, 25 bp, Artificial Sequence
  Description of Artificial Sequence Synthetic oligonucleotide
tgaggggcaagcaacatcgatcggt

SEQ ID NO: 16 - DNA, 35 bp, Artificial Sequence
  Description of Artificial Sequence Synthetic oligonucleotide
tgaggggcagnnnnnnnnnnnnnnnnnnnnnnnnn

SEQ ID NO: 17 - DNA, 35 bp, Artificial Sequence
  Description of Artificial Sequence Synthetic oligonucleotide
tgaggggcnnnnnnnnnnnnnnnnnnnnnnnnnnn

SEQ ID NO: 18 - DNA, 35 bp, Artificial Sequence
  Description of Artificial Sequence Synthetic oligonucleotide
tgagggnnnnnnnnnnnnnnnnnnnnnnnnnnnnn

SEQ ID NO: 19 - DNA, 35 bp, Artificial Sequence
  Description of Artificial Sequence Synthetic oligonucleotide
tgagnnnnnnnnnnnnnnnnnnnnnnnnnnnnnnn

SEQ ID NO: 20 - DNA, 34 bp, Artificial Sequence
  Description of Artificial Sequence Synthetic oligonucleotide
cgggaggaaggctagctacaacgagaggcgttgt

SEQ ID NO: 21 - DNA, 34 bp, Artificial Sequence
  Description of Artificial Sequence Synthetic oligonucleotide
cgggaggaaggctagctacaacaagaggcgttgt

SEQ ID NO: 22 - DNA, 25 bp, Artificial Sequence
  Description of Artificial Sequence Synthetic oligonucleotide
cgggaggaaagcaacatcgatcggt

SEQ ID NO: 23 - DNA, 34 bp, Artificial Sequence
  Description of Artificial Sequence Synthetic oligonucleotide
cgggaggaaggctagctacaagcagagggcttgt

SEQ ID NO: 24 - DNA, 34 bp, Artificial Sequence
  Description of Artificial Sequence Synthetic oligonucleotide
cgggaggaaggctagcacacagagggtcatggtt

SEQ ID NO: 25 - DNA, 33 bp, Artificial Sequence
  Description of Artificial Sequence Synthetic oligonucleotide
cgggaggaaggctagctacaacgagaggcgttg

SEQ ID NO: 26 - DNA, 34 bp, Artificial Sequence; identical to SEQ ID NO: 20
  Description of Artificial Sequence Synthetic oligonucleotide

SEQ ID NO: 27 - DNA, 34 bp, Artificial Sequence
  Description of Combined DNA/RNA Molecule Synthetic oligonucleotide
cgggaggaaggctagctacaacgagaggcgtugt

SEQ ID NO: 28 - DNA, 33 bp, Artificial Sequence; identical to SEQ ID NO: 25
  Description of Artificial Sequence Synthetic oligonucleotide

SEQ ID NO: 29 - DNA, 33 bp, Artificial Sequence; identical to SEQ ID NO: 25
  Description of Artificial Sequence Synthetic oligonucleotide

SEQ ID NO: 30 - DNA, 32 bp, Artificial Sequence
  Description of Artificial Sequence Synthetic oligonucleotide
gggaggaaggctagctacaacgagaggcgttt

SEQ ID NO: 31 - DNA, 34 bp, Artificial Sequence
  Description of Artificial Sequence Synthetic oligonucleotide
cgggaggaaggctacctacaacgagaggcgttgt

SEQ ID NO: 32 - DNA, 33 bp, Artificial Sequence
  Description of Artificial Sequence Synthetic oligonucleotide
tgggagccaggctagctacaacgaagcgaggct

SEQ ID NO: 33 - DNA, 20 bp, Artificial Sequence
  Description of Artificial Sequence Synthetic oligonucleotide
cgggaggaacgaggcgttgt

SEQ ID NO: 34 - DNA, 25 bp, Artificial Sequence
  Description of Artificial Sequence Synthetic oligonucleotide
cgggaggaaggctagctacaacgat

SEQ ID NO: 35 - DNA, 34 bp, Artificial Sequence; identical to SEQ ID NO: 20
  Description of Artificial Sequence Synthetic oligonucleotide

SEQ ID NO: 36 - DNA, 34 bp, Artificial Sequence; identical to SEQ ID NO: 20
  Description of Artificial Sequence Synthetic oligonucleotide

SEQ ID NO: 37 - DNA, 34 bp, Artificial Sequence; identical to SEQ ID NO: 20
  Description of Artificial Sequence Synthetic oligonucleotide

SEQ ID NO: 38 - DNA, 33 bp, Artificial Sequence
  Description of Artificial Sequence Synthetic oligonucleotide
caacgcctctcgttgtagctagccttcctcccg

SEQ ID NO: 39 - DNA, 30 bp, Artificial Sequence
  Description of Artificial Sequence Synthetic oligonucleotide
cgggaggaagnnnnnnnnnnnnnnnnnnnn

SEQ ID NO: 40 - DNA, 35 bp, Artificial Sequence
  Description of Artificial Sequence Synthetic oligonucleotide
cgggaggaagnnnnnnnnnnnnnnnnnnnnnnnnn

SEQ ID NO: 41 - DNA, 34 bp, Artificial Sequence
  Description of Artificial Sequence Synthetic oligonucleotide
cgggaggaagnnnnnnnnnnnnnnnnnnnnnnnt

SEQ ID NO: 42 - DNA, 34 bp, Artificial Sequence
  Description of Artificial Sequence Synthetic oligonucleotide
cgggaggaagtagtagtagtagtagtagtagtag

SEQ ID NO: 43 - DNA, 15 bp, Artificial Sequence
  Description of Artificial Sequence Synthetic oligonucleotide
cgggaggaagnnnnn

SEQ ID NO: 44 - DNA, 20 bp, Artificial Sequence
  Description of Artificial Sequence Synthetic oligonucleotide
cgggaggaagnnnnnnnnnn

SEQ ID NO: 45 - DNA, 25 bp, Artificial Sequence
  Description of Artificial Sequence Synthetic oligonucleotide
cgggaggaagnnnnnnnnnnnnnnn

SEQ ID NO: 46 - DNA, 33 bp, Artificial Sequence; identical to SEQ ID NO: 25
  Description of Artificial Sequence Synthetic oligonucleotide

SEQ ID NO: 47 - DNA, 35 bp, Artificial Sequence
  Description of Artificial Sequence Synthetic oligonucleotide
cgggaggannnnnnnnnnnnnnnnnnnnnnnnnnn

SEQ ID NO: 48 - DNA, 35 bp, Artificial Sequence
  Description of Artificial Sequence Synthetic oligonucleotide
cgggagnnnnnnnnnnnnnnnnnnnnnnnnnnnnn

SEQ ID NO: 49 - DNA, 35 bp, Artificial Sequence
  Description of Artificial Sequence Synthetic oligonucleotide
cgggnnnnnnnnnnnnnnnnnnnnnnnnnnnnnnn

SEQ ID NO: 50 - DNA, 34 bp, Artificial Sequence
  Description of Artificial Sequence Synthetic oligonucleotide
cgggaggaagaaaaaaaaaaaaaaaaaaaaaaat

SEQ ID NO: 51 - DNA, 35 bp, Artificial Sequence
  Description of Artificial Sequence Synthetic oligonucleotide
cgggaggaaghhhhhhhhhhhhhhhhhhhhhhhhh

SEQ ID NO: 52 - DNA, 34 bp, Artificial Sequence
  Description of Artificial Sequence Synthetic oligonucleotide
cgggaggaagtaggattaggattaggattaggat

SEQ ID NO: 53 - DNA, 10 bp, Artificial Sequence
  Description of Artificial Sequence Synthetic oligonucleotide
cgggaggaat

SEQ ID NO: 54 - DNA, 35 bp, Artificial Sequence
  Description of Artificial Sequence Synthetic oligonucleotide
gggaggaaagnnnnnnnnnnnnnnnnnnnnnnnnn

SEQ ID NO: 55 - DNA, 30 bp, Artificial Sequence
  Description of Artificial Sequence Synthetic oligonucleotide
gggaggaaagnnnnnnnnnnnnnnnnnnnn

SEQ ID NO: 56 - DNA, 25 bp, Artificial Sequence
  Description of Artificial Sequence Synthetic oligonucleotide
gggaggaaagnnnnnnnnnnnnnnn

SEQ ID NO: 57 - DNA, 20 bp, Artificial Sequence
  Description of Artificial Sequence Synthetic oligonucleotide
gggaggaaagnnnnnnnnnn

SEQ ID NO: 58 - DNA, 15 bp, Artificial Sequence
  Description of Artificial Sequence Synthetic oligonucleotide
gggaggaaagnnnnn

SEQ ID NO: 59 - DNA, 24 bp, Artificial Sequence
  Description of Artificial Sequence Synthetic oligonucleotide
agggagggaggaagggagggaggg

SEQ ID NO: 60 - DNA, 28 bp, Artificial Sequence
  Description of Artificial Sequence Synthetic oligonucleotide
agggagggaggaagggagggagggaggg

SEQ ID NO: 61 - DNA, 32 bp, Artificial Sequence
  Description of Artificial Sequence Synthetic oligonucleotide
agggagggaggaagggagggagggagggaggg

SEQ ID NO: 62 - DNA, 24 bp, Artificial Sequence
  Description of Artificial Sequence Synthetic oligonucleotide
agggagggagggagggagggaggg

SEQ ID NO: 63 - DNA, 20 bp, Artificial Sequence
  Description of Artificial Sequence Synthetic oligonucleotide
agggagggaggaagggaggg

SEQ ID NO: 64 - DNA, 16 bp, Artificial Sequence
  Description of Artificial Sequence Synthetic oligonucleotide
agggagggaggaaggg

SEQ ID NO: 65 - DNA, 33 bp, Artificial Sequence
  Description of Artificial Sequence Synthetic oligonucleotide
gagggggaaggctagctacaacgaagttcgtcc

SEQ ID NO: 66 - DNA, 33 bp, Artificial Sequence
  Description of Artificial Sequence Synthetic oligonucleotide
gcggggacaggctagctacaacgacagctgcat

SEQ ID NO: 67 - DNA, 33 bp, Artificial Sequence
  Description of Artificial Sequence Synthetic oligonucleotide
ggggcaggaagcaacatcgatcgggacttttga

SEQ ID NO: 68 - DNA, 36 bp, Artificial Sequence
  Description of Artificial Sequence Synthetic oligonucleotide
acgggaggaaggctagctacaacgagaggcgttgat

SEQ ID NO: 69 - DNA, 38 bp, Artificial Sequence
  Description of Artificial Sequence Synthetic oligonucleotide
gacgggaggaaggctagctacaacgagaggcgttgagt

SEQ ID NO: 70 - DNA, 18 bp, Artificial Sequence
  Description of Artificial Sequence Synthetic oligonucleotide
ttggagggggtggtgggg

SEQ ID NO: 71 - DNA, 29 bp, Artificial Sequence
  Description of Artificial Sequence Synthetic oligonucleotide
tttggtggtggtggttgtggtggtggtgg

SEQ ID NO: 72 - DNA, 20 bp, Artificial Sequence
  Description of Artificial Sequence Synthetic oligonucleotide
ctggaggaacgaggcgttgt

SEQ ID NO: 73 - DNA, 16 bp, Artificial Sequence
  Description of Artificial Sequence Synthetic oligonucleotide
caacgagaggcgttgt

SEQ ID NO: 74 - DNA, 34 bp, Artificial Sequence
  Description of Artificial Sequence Synthetic oligonucleotide
ccgctgccaggctagctacaacgacccggacgtt

SEQ ID NO: 75 - DNA, 34 bp, Artificial Sequence
  Description of Artificial Sequence Synthetic oligonucleotide
gccagccgcggctagctacaacgatggctccact

SEQ ID NO: 76 - DNA, 34 bp, Artificial Sequence
  Description of Artificial Sequence Synthetic oligonucleotide
gcgacgtgaggctagctacaacgagtggaggagt

SEQ ID NO: 77 - DNA, 18 bp, Artificial Sequence
  Description of Artificial Sequence Synthetic oligonucleotide
gtgccggggtcttcgggc

SEQ ID NO: 78 - DNA, 18 bp, Artificial Sequence
  Description of Artificial Sequence Synthetic oligonucleotide
tctcccagcgtgcgccat

SEQ ID NO: 79 - DNA, 34 bp, Artificial Sequence
  Description of Artificial Sequence Synthetic oligonucleotide
ctggaggaaggctagctacaacgagaggcgttgt

SEQ ID NO: 80 - DNA, 30 bp, Artificial Sequence
  Description of Artificial Sequence Synthetic oligonucleotide
ggaggaaggctagctacaacgagaggcgtt

SEQ ID NO: 81 - DNA, 25 bp, Artificial Sequence
  Description of Artificial Sequence Synthetic oligonucleotide
ttagggttagggttagggttagggt

SEQ ID NO: 82 - DNA, 16 bp, Artificial Sequence
  Description of Artificial Sequence Synthetic oligonucleotide
tcctggcggggaagtt

SEQ ID NO: 83 - DNA, 27 bp, Artificial Sequence
  Description of Artificial Sequence Synthetic oligonucleotide
tgtttgtttgtttgtttgtttgtttgt

SEQ ID NO: 84 - DNA, 36 bp, Artificial Sequence
  Description of Artificial Sequence Synthetic oligonucleotide
aagaggtggtggaggaggtggtggaggaggtggagg

SEQ ID NO: 85 - DNA, 16 bp, Artificial Sequence
  Description of Artificial Sequence Synthetic oligonucleotide
ggtgggtggtggtggg

SEQ ID NO: 86 - DNA, 15 bp, Artificial Sequence
  Description of Artificial Sequence Synthetic oligonucleotide
ggctagctacaacga

SEQ ID NO: 87 - DNA, 15 bp, Artificial Sequence
  Description of Artificial Sequence Synthetic oligonucleotide
agcaacatcgatcgg

SEQ ID NO: 88 - DNA, 15 bp, Artificial Sequence
  Description of Artificial Sequence Synthetic oligonucleotide
ggctasctacaacga

SEQ ID NO: 89 - DNA, 15 bp, Artificial Sequence
  Description of Artificial Sequence Synthetic oligonucleotide
agcaacatcsatcgg

SEQ ID NO: 90 - DNA, 23 bp, Artificial Sequence
  Description of Artificial Sequence Synthetic oligonucleotide
ggctagctacaacgagaggcgtt

SEQ ID NO: 91 - DNA, 21 bp, Artificial Sequence
  Description of Artificial Sequence Synthetic oligonucleotide
ggctagctacaacgacgttgc

SEQ ID NO: 92 - DNA, 24 bp, Artificial Sequence
  Description of Artificial Sequence Synthetic oligonucleotide
ggctagctacaacgacagctgcat

SEQ ID NO: 93 - DNA, 28 bp, Artificial Sequence
  Description of Artificial Sequence Synthetic oligonucleotide
gaaggctagctacaacgaagttcgtcct

SEQ ID NO: 94 - DNA, 27 bp, Artificial Sequence
  Description of Artificial Sequence Synthetic oligonucleotide
tcaggctagctacaacgacgtggtggt

SEQ ID NO: 95 - DNA, 28 bp, Artificial Sequence
  Description of Artificial Sequence Synthetic oligonucleotide
gaaggctacctacaacgagaggcgttgt

SEQ ID NO: 96 - DNA, 27 bp, Artificial Sequence
  Description of Artificial Sequence Synthetic oligonucleotide
gcaggctagctacaacgacgtcgtgac

SEQ ID NO: 97 - DNA, 20 bp, Artificial Sequence
  Description of Artificial Sequence Synthetic oligonucleotide
gctagctacaacgacgtcgc

SEQ ID NO: 98 - DNA, 24 bp, Artificial Sequence
  Description of Artificial Sequence Synthetic oligonucleotide
caggctagctacaacgacgtcgcg

SEQ ID NO: 99 - DNA, 26 bp, Artificial Sequence
  Description of Artificial Sequence Synthetic oligonucleotide
gcaggctagctacaacgacgtcgcgg

SEQ ID NO: 100 - DNA, 27 bp, Artificial Sequence
  Description of Artificial Sequence Synthetic oligonucleotide
gcaagcaacatcgatcggcgtcgtgac

SEQ ID NO: 101 - DNA, 10 bp, Artificial Sequence
  Description of Artificial Sequence Synthetic oligonucleotide
cgggaggaag

SEQ ID NO: 102 - DNA, 10 bp, Artificial Sequence
  Description of Artificial Sequence Synthetic oligonucleotide
gggaggaaag

(19) United States
(12) Patent Application Publication
(10) Pub. No.: US 2009/0312399 A1
(43) Pub. Date: Dec. 17, 2009

(54) GUANOSINE-RICH OLIGONUCLEOTIDES AS AGENTS FOR INDUCING CELL DEATH IN EUKARYOTIC CELLS

(75) Inventors: Laurent Pierre Rivory, Springwood (AU); Donald John Birkett, Mosman (AU)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: JOHNSON & JOHNSON RESEARCH PTY, LTD, Eveleigh (AU)

(21) Appl. No.: 11/993,537

(22) PCT Filed: Jun. 28, 2006
(86) PCT No.: PCT/IB06/02887

§ 371 Date: Dec. 21, 2007

Related U.S. Application Data
(60) Provisional application No. 60/694,745, filed on Jun. 28, 2005.

(51) Int. Cl. A61K 31/7088; C07H 21/02; C07H 21/04; C12N 5/02

(52) U.S. Cl. 514/44 R; 536/23.1; 435/375

(57) ABSTRACT

The present invention relates to guanosine-rich oligonucleotides having the capacity to induce cell death, having characteristics of programmed cell death, in non-quiescent cells of higher eukaryotic organisms. The invention also relates to therapeutic methods involving the administration of these nucleic acid molecules to subjects suffering from, or being predisposed to, disorders involving abnormal cell proliferation and migration. The invention also concerns pharmaceutical compositions comprising the guanosine-rich nucleic acid molecules, in association with suitable carriers.

### FIELD OF THE INVENTION

The present invention relates to guanosine-rich oligonucleotides having the capacity to induce cell death, having characteristics of programmed cell death, in non-quiescent cells of higher eukaryotic organisms. The invention also relates to therapeutic methods involving the administration of these nucleic acid molecules to subjects suffering from, or being predisposed to, disorders involving abnormal cell proliferation and migration. The invention also concerns pharmaceutical compositions comprising the guanosine-rich nucleic acid molecules, in association with suitable carriers.

### BACKGROUND AND PRIOR ART

Oligonucleotide (ON) drugs are nucleic acid molecules having therapeutic utility. They vary widely in composition, and bring about their biological responses in many different ways.

A first class of ON drugs has been actively developed to target gene-specific RNA sequences. These ON drugs generally demonstrate a complete or near-complete degree of complementarity with the target sequence. For example, catalytic nucleic acid molecules such as ribozymes, minizymes and DNAzymes, act by specifically binding to and cleaving the target RNA molecule in the cell. Antisense ONs form RNA:DNA heteroduplexes with their targets and may then trigger RNA degradation through activation of RNAseH or produce translational arrest.

Other types of synthetic ONs having therapeutic potential include double-stranded RNA (RNAi), and nucleic acid transcriptional decoys.

It has become clear over the years that many of the actions of synthetic ONs can be mediated by “pleiotropic”, non-antisense mechanisms. These include but are not limited to the binding of oligonucleotides, particularly those that have phosphorothioate-modified inkages, to heparin-binding proteins such as bFGF (Guvakova et al., 1995). This occurs largely through electrostatic interactions. Because oligonucleotides can self-assemble into complex tertiary structures (Wyatt et al., 1996), these effects may be highly sequence-specific. Indeed, synthetic ONs can be screened for their ability to bind to specific ligands. These so-called “aptamer” ONs may be useful therapeutically (Burgstaller et al., 2002).

Synthetic ONs may also bind to specific receptors involved in innate response. Specifically, unmethylated CpG motifs are relatively rare in eukaryotes but common in bacteria and are sensed as foreign DNA by toll-like receptor 9 (TLR-9) (Hemmi et al., 2000). In B-cells, stimulation of TLR-9 triggers a cascade culminating in the secretion of cytokines including TNF-alpha and IL-6. Subtle changes in the DNA sequences flanking the CpG motif and formation of tertiary structures (Wu et al., 2004) can dramatically affect both the magnitude of the response and the specific profile of cytokines involved. CpG and CpG-mimicking ONs may be of use therapeutically as adjuvants for vaccination.

Some ONs that are rich in guanosine bases have been shown to be able to block CpG activation of B-cells in cell culture (Lenert et al., 2001). These “inhibitory” ONs do not appear to have any effect on cells in isolation and only serve to reduce or inhibit cell response to CpG motifs.

Other G-rich ONs have been described that can inhibit cell proliferation significantly (Yaswen et al., 1993). This may be due in part to their ability to form G-quadruplexes in which alignment of G-rich strands results in the formation of coordinated guanosine tetrads. These quadruplexes may bind metal ions and DNA etc. In some cases, these quadruplexes are important for aptameric properties, such as the binding of the protein nucleolin (Jueliger and Bates, 2004).

There thus exists a considerable number of different types of synthetic oligonucleotides, capable of exerting a range of potentially therapeutic effects on cells of living organisms. However, in spite of this significant source of active molecules, it is not always straightforward to efficiently exploit these ONs when seeking therapeutic agents for a given pathology. Indeed, ONs such as antisense, ribozymes and DNAzymes, require complementarity with the target, and therefore in disease contexts where the precise target RNA is unknown, this type of technology is not readily applicable.

Moreover, the above-mentioned “pleiotropic” effects of synthetic oligonucleotides on cells, whilst being of great potential use therapeutically, depend to a large extent on the composition of the oligonucleotide and the system in which they are tested. Prediction of specific activities in cellular systems is difficult to make on the basis of sequence identity and the biological activity of each oligonucleotide needs to be evaluated on a case by case basis (Benimetskaya, 1997). This is explained by the fact that such oligonucleotides display a high degree of polymorphism. Also, the identities of many oligonucleotide-binding cellular proteins remain unknown. A rational approach to ON drug design based on pleiotropic effects is therefore not always feasible.

ON-drug treatment of disorders associated with abnormal cell proliferation is particularly challenging. Indeed, factors involved in cell-cycle progression and de-regulation are numerous and interactions are complex. Knowledge of potential cellular targets is to date still incomplete for many pathologies. In addition, conditions involving aberrant cell proliferation often respond more readily to cytotoxic therapy rather than cytostatic therapy. Consequently it is desirable to develop ON drugs which induce cell death rather than simply inhibiting cell proliferation. The cytotoxic effect must however be specific for the abnormally proliferating cells. The design of ON drugs for treatment of disorders involving aberrant cell proliferation can therefore be more complex than in areas where a defined target is involved.

For example, it has recently been reported that certain molecules belonging to the DNAzyme family of ONs inhibit vascular muscle and endothelial cell proliferation. In particular, workers investigating the properties of DNAzymes targeted to c-Jun mRNA showed that some of these ONs can cleave synthetic c-Jun mRNA in vitro. Inhibition, by such DNAzymes, of serum-inducible proliferation of human and porcine primary vascular smooth muscle cells (Khachigian et al, 2002), and human microvascular endothelial cells in vitro has also been shown (International patent application WO 03/072114; Zhang et al., 2004). The DNAzymes investigated did not all show anti-proliferative effects. It is reported that some of the c-Jun DNAzymes stimulated proliferation of smooth muscle cells, whilst others, which were able to cleave synthetic c-Jun mRNA in vitro, failed to modulate smooth muscle cell proliferation in either rat or human cells. Similarly, some catalytically active c-Jun DNAzymes were found to have little effect on proliferation of human microvascular endothelial cells. The authors conclude that mRNA cleavage alone is not a reliable performance indicator of DNAzyme efficacy in a biological system (Zhang et al., 2004). The capacity of the oligonucleotides to induce cell death was not investigated by these workers.

DNAzymes targeting c-myc oncogene mRNA have -also been reported to cleave synthetic c-myc mRNA In vitro. Inhibition of smooth muscle cell proliferation in rat SV40LT-SMC cell lines has also been observed with these agents (Sun et al., 1999). Inhibition of proliferation of human smooth muscle cells and induction of cell death was not reported.

There thus remains a need for ON molecules which are specific inducers of cell death in proliferating cells. Desirable molecules are suitable for use as therapeutic agents in the treatment and prevention of disorders involving aberrant cell proliferation, and for the manufacture of medicaments for use in such disorders.

It is an object of the present invention to provide such oligonucleotide molecules. In particular, it is an object of the present invention to provide a class of oligonucleotide molecules which induce cell death in proliferating cells of higher eukaryotic organisms. It is also an object of the invention to provide a class of oligonucleotide molecules which specifically induce cell death in proliferating cells without producing detrimental effects in non-proliferating cells.

These and other objects are achieved by the present invention as evidenced by the summary of the invention, description of the preferred embodiments and the claims.

### SUMMARY OF THE INVENTION

The aims of the invention are met by a new class of G-rich oligonucleotides having a novel combination of unique 5′ region sequences and total length requirements.

The present invention relates to a class of DNA-containing oligonucleotides characterized by a length of 20 to 50 nucleotides, for example 21 to 50, or 25 to 50 nucleotides, and a guanosine-rich region, constituting the 5′ segment of the molecule. The G-rich region has a length of from 6 to 9 nucleotides, and contains a purine tract comprising at least 4 consecutive purine nucleotides. Within the G-rich region, there is a triple G motif (G-G-G), the 5′ extremity of the triple G motif being positioned no more than three nucleotides from the 5′ extremity of the oligonucleotide. The 3′ region of the oligonucleotides can be essentially any nucleotide sequence, there being no particularly rigid sequence requirements in this part of the molecule. According to the invention, these oligonucleotides, which have been found to induce cell death having features typical of programmed cell death in dividing cells, are used in methods of treatment of disorders involving aberrant proliferation of cells, and in the preparation of medicaments for the treatment of such disorders.

### BRIEF DESCRIPTION OF THE DRAWINGS

FIG. 1 is a graph showing cytotoxic activity of several oligonucleotides against HMEC-1 cells when transfected with Fugene6 and assayed with Cell Titer Blue Assay. Fugene6 alone had no activity (not shown). Only those with the required 5′ G-rich sequence are active over the concentration range of 0-100 nM.

Key: Open square: Oligo 1;

Open triangle: Oligo 2;

Inverted open triangle: Oligo 3;

Open circle: Oligo 4;

Cross: Oligo 5;

Star: Oligo 6.

FIG. 2 is a graph showing the fully phosphorothioate modified bcl-2 and c-myb antisense molecules do not have concentration-dependent cytotoxicity to HMEC-1 cells of the sort demonstrated in FIG. 1.

Key: Open square: c-myb antisense (Oligo 32);

Open triangle: bcl-2 antisense (Oligo 33).

FIG. 3 is a graph illustrating the co-incubation of HMEC-1 cells with chloroquine (100 μM) does not block the activity of Oligo 4. Chloroquine is an inhibitor of endosomal maturation and TLR-9 signalling. TLR-9 signalling is involved in the biological activity of CpG oligonucleotides.

Key: Open square: Oligo 4+chloroquine;

Open circle: Oligo 4.

FIG. 4 is a graph illustrating oligonucleotides with either methyl cytosines in the CpG dinucleotide sequences (Oligo 9) or GpC sequences (i.e., reverse sequences, Oligo 10) are also cytotoxic to HMEC-1 cells.

Key: Open diamond: ^{m}CpG (Oligo 9);

Inverted Open triangle: Oligo 10.

FIG. 5 is a graph showing co-transfection of HMEC-1 cells with the so-called “inhibitory” oligonucleotides Oligo 36 or Oligo 37 does not inhibit the cytotoxic activity of Oligo 4. Furthermore, these oligonucleotides are not cytotoxic in their own right over the relevant range of concentrations.

Key: Open square: Oligo 4;

Open triangle: Oligo 36;

Inverted open triangle: Oligo 37;

Open diamond: Oligo 4+Oligo 36 (100 nM)

Open circle: Oligo 4+Oligo 37 (100 nM).

FIG. 6 is a bar graph illustrating HMEC-1 cell survival at 0.2 μM. Oligonucleotides without the requisite G-triplet in the 5′ terminus region (e.g. Oligos 34 and 35) have no cytotoxic activity even at concentrations as high as 200 nM.

FIG. 7 is a graph illustrating oligonucleotides in which the G-triplet has been modified through the substitution of one of the 3 consecutive guanosines with 7 deaza-guanosine have greatly reduced cytotoxic activity against HMEC-1 cells.

Key: Open square: Oligo 38;

Open triangle: Oligo 39;

Inverted open triangle: Oligo 40;

Open circle: Oligo 4.

FIG. 8 is a graph showing the cytotoxicity of pooled, synthetic, random-tailed oligonucleotides as a function of their overall length. All oligonucleotides shared the same sequence for the first 10 bases.

Key: Open square: CGGGAGGMG(N₅) (Oligo 41)

Open triangle: CGGGAGGAAG(N₁₀) (Oligo 42);

Inverted open triangle: CGGGAGGAAG(N₁₅) (Oligo 43)

Open diamond: CGGGAGGAAG(N₂₀) (Oligo 12)

Cross: CGGGAGGMG(N₂₅) (Oligo 13)

FIG. 9 is a graph illustrating the cytotoxic activity of Oligo 4 against several cell lines in culture when treated as for the HMEC-1 cells.

Key: Open square: 3T3;

Open triangle: Hela;

Inverted open triangle: HEK 293;

Open diamond: CaSki

Open circle: A549;

Cross: HMEC1;

Star: MDA-MB231.

FIG. 10 is a graph showing the cytotoxic activity of analogues of Oligo 1 in which phosphorothioate linkages have been introduced at the 5′ and 3′ ends of the molecule. Complete back-bone substitution greatly suppresses cytotoxic activity.

Key: Open square: 9+9 PS (Oligo 16);

Open triangle: 7+7 PS (Oligo 17);

Inverted open triangle: 5+5 PS (Oligo 18);

Open diamond: All PS (Oligo 44);

FIG. 11 is a graph illustrating the cytotoxicity of analogues of Oligo 1 with. terminal inverted bases against HMEC-1 cells.

Key: Open circle: 3′-3′C (Oligo 1)

Open square: 5′-5′T (Oligo 14)

Open triangle: 3′-3′C+5′-5′T (Oligo 15);

FIG. 12 is a graph showing the cytotoxic activity of analogues of Oligo 4. A totally unmodified phosphodiester oligonucleotide has comparable activity, whereas the introduction of 2′-O-methyl ribose modifications appears to reduce activity.

Key: Open square: 2+2 2′O Methyl (Oligo 20)

Open triangle: unmodified (Oligo 19);

Open circle: 3′-3′T (Oligo 4)

FIG. 13 is a graph illustrating the addition of bulky substitutions at the 3′ terminus of active oligonucleotides does not greatly diminish the cytotoxicity towards HMEC-1 cells.

Key: Open square: Oligo 4 3′ cholesteryl (Oligo 11)

Open circle: Oligo 4

FIG. 14 is a bar graph showing HMEC-1 Cell Cycle Profile. The addition of an active oligonucleotide (Oligo 4) to HMEC-1 cells appears not to cause cell-cycle arrest or accumulation of any particular cycle phase, as compared to the non-cytotoxic Oligo 7. Note, however, the increase in sub-G₀ cells, indicating that these have died.

Key: Non-shaded open bar: Sub G₀

Spotted bar: G₀/G₁

Horizontally striped bar: S

Vertically striped bar: G2/M

FIG. 15 are slides showing staining of HMEC-1 cells with propidium iodide (vertical axis) and Annexin V (horizontal axis) after treatment with Oligo 4 and Oligo 7 at 100 nM. The top row of slides shows 24 h post-transfection, and the bottom row shows 48 h post-transfection.

FIG. 16 is a bar graph illustrating the activation of caspases in HMEC-1 cells following treatment with Oligo 4 as a function of time. HMEC-1 cells were harvested and analysed by FACS after staining with a pan-caspase substrate.

Key: Non-shaded open bar: 24 hours

Shaded bar: 48 hours

FIG. 17 are slides showing the activation of caspase 8 in similar conditions to FIG. 16 at 48 hours post-transfection.

FIG. 18 is a graph illustrating the cytotoxic activity of purine-only oligonucleotides in HMEC-1 cells after transfection with Fugene6.

Key: Open square: Oligo 77

Open triangle: Oligo 79

Open diamond: Oligo 80

Cross: Oligo 81

Inverted open triangle: Oligo 82

FIG. 19. is a graph illustrating the cytotoxic activity of sequence variants of Oligo 4 with single base changes. Oligo 47 is predicted to self-hybridize at the 5′ end, whereas Oligo 27 is predicted to fold in the same manner as Oligo 4, in a way that does not involve the terminal 5′ region.

Key: Open triangle: Oligo 27

Inverted open triangle: Oligo 4

Open circle: Oligo 47

FIG. 20 is a graph showing the cytotoxicity of analogues of Oligo 1 in HMEC-1 cells.

Key: Open square: Oligo 23

Open triangle: Oligo 24

Inverted open triangle: Oligo 25

Open diamond: Oligo 1

FIG. 21 is a graph illustrating the further demonstration of active and inactive oligonucleotides when tested on HMEC-1 cell cultures:

Key: Open triangle: Oligo 48

Open diamond: Oligo 26

FIG. 22 is a graph showing the further demonstration of active and inactive oligonucleotides when tested on HMEC-1 cell cultures.

Key: Open square: Oligo 27

Open triangle: Oligo 50

Inverted open triangle: Oligo 51

Open diamond: Oligo 52

FIG. 23 is a graph showing the further demonstration of active and inactive oligonucleotides when tested on HMEC-1 cell cultures.

Key: Open triangle: Oligo 53

Inverted open triangle: Oligo 28

Open diamond: Oligo 54

Open circle: Oligo 1

FIG. 24 is a graph showing the further demonstration of active and inactive oligonucleotides when tested on HMEC-1 cell cultures.

Key: Open square: Oligo 1

Open triangle: Oligo 55

Inverted open triangle: Oligo 56

Open diamond: Oligo 57

FIG. 25 is a graph illustrating the further demonstration of active and inactive oligonucleotides when tested on HMEC-1 cell cultures.

Key: Inverted open triangle: Oligo 29

Open diamond: Oligo 30

Open circle: Oligo 58

FIG. 26 is a graph showing the further demonstration of active and inactive oligonucleotides when tested on HMEC-1 cell cultures. Note the activity of the 5′phosphorylated oligonucleotide.

Key: Open square: Oligo 59

Inverted open triangle: Oligo 60

Open diamond: Oligo 31

FIG. 27 is a graph showing the cytotoxicity of Oligo 4 as single stranded (ss) DNA and as a double-stranded (ds) duplex with its complementary sequence (Oligo 61). The duplex was annealed in vitro prior to transfection as per normal.

Key: Shaded triangle: ss Oligo 4

Shaded square: ss complement of Oligo 4

Inverted shaded triangle: ds Oligo 4

FIG. 28 is a graph showing the influence of the length of the defined G-rich region on the cytotoxicity in HMEC-1 cells.

Key: Shaded square: Oligo 66

Shaded triangle: Oligo 67

Inverted shaded triangle: Oligo 68

Shaded diamond: Oligo 69

Shaded circle: Oligo 25

FIG. 29 is a graph showing the influence of cell density and contact inhibition on the cytotoxicity of the oligonucleotides. Curves labeled “high” show the % survival of ARPE cells (human retinal pigmented epithelium) seeded at “high” densities (50,000 cells per well), 48 hours after transfection with the indicated oligonucleotides. Cells seeded at high densities rapidly reach a state of contact-inhibited quiescence. Curves labeled “low” show the % survival of ARPE cells seeded at “low” densities (4,000 cells per well), 48 hours after transfection with the same oligonucleotides. Cells seeded at low densities do not reach quiescence and continue to actively divide. The cytotoxic effect is abolished (at concentrations <0.2 microM) for cells that are quiescent. Cytotoxic oligonucleotides of the invention thus have potent activity against dividing cells but no appreciable activity against quiescent cells.

Key: Shaded square: Oligo 7 “high”

Shaded triangle: Oligo 4 “high”

Inverted shaded triangle: Oligo 7 “low”

Shaded diamond: Oligo 4 “low”

FIG. 30 are slides showing the mitochondrial depolarization with active and inactive oligonucleotides. A: JC-1 assessment of Ψm. When incubated at a concentration of 100 nM for 48 hours, Oligo 4 caused a large green shift in fluorescence of cells labeled with JC-1. Taxol (1 μM) was used as a positive control. B: is a bar graph showing the percentage of cells with depolarized mitochondria as a function of time of incubation with the oligonucleotides. All oligonucleotides were used at a concentration of 100 nM.

Key: Non-shaded open bar: mock

Stippled bar: Oligo 7

Fully Shaded bar: Oligo 4.

### DETAILED DESCRIPTION OF THE PREFERRED EMBODIMENTS

In the context of the present invention, the following terms have the following meanings:
Oligonucleotide: the term “oligonucleotide” (ON) refers to a polymer of single- or double-stranded nucleotides, having a relatively short length. In the context of the invention, “oligonucleotide” and its grammatical equivalents includes the full range of nucleic acids.
ODN: the term “ODN” signifies “oligodeoxynucleotide” i.e. a DNA-containing oligonucleotide. In the context of the invention, ODNs consist exclusively of deoxyribonucleotides, or comprise predominantly deoxyribonucleotides. Substitution of one or more deoxyribonucleotides by corresponding ribonucleotides or other nucleotide analogues and/or derivatives may be made, provided the cytotoxic properties of the ODN are not thereby adversely affected.
ODN of the invention: an ODN of the invention is an oligodeoxynucleotide consisting of a 5′ G-rich region and a 3′ tail region contiguous to the G-rich region, wherein the G-rich region meets the structural definition set out in at least one of the formulae 1 to 7 (as defined herein), and the said oligonucleotide has the capacity to induce cell death, having characteristics of programmed cell death, in cells of at least two higher eukaryotic organisms of different species. According to the invention, reference to Formulae 1 to 7 includes any or all of the following formulae as defined herein:

$$Formulae \text{:} \{ \begin{matrix}1 , 1 a ; \\ 2 , 3 , 4 , 5 , \\ 5.1 , 5.1 a , 5.1 b , \\ ( 5.1 .1 ) ; ( 5.1 .2 ) ; ( 5.1 .3 ) ; ( 5.1 .3 b ) ; ( 5.1 .4 ) ; ( 5.1 .4 b ) ; ( 5.1 .5 ) ; \\ 5.2 ; \\ 6 ; \\ 6.1 , ( 6.1 .1 ) ; ( 6.1 .2 ) ; ( 6.1 .3 ) ; ( 6.1 .4 ) ; ( 6.1 .5 ) ; \\ 6.2 ; \\ 7 , 7.1 , 7.2 , 7.3 , 7.4 , 7.5 , 7.6 , 7.7 , 7.8 , 7.9 , 7.10 .\end{matrix}$$

Purine base: nitrogenous heterocyclic base consisting of a six-membered and a five-membered nitrogen-containing ring, fused together. Adenine and guanine are the principal purine bases incorporated into nucleic acids.
Pyrimidine base: nitrogenous heterocyclic base consisting of a six-membered nitrogen-containing ring. Uracil, thymine and cytosine are the principal pyrimidine bases incorporated into nucleic acids.
Nucleoside: a compound consisting of a purine or pyrimidine base covalently linked to a pentose, usually ribose in ribonucleosides, and 2-deoxyribose in deoxyribonucleosides. Nucleosides containing the bases adenine, guanine, cytosine, uracil, thymine and hypoxanthine are referred to, respectively, as (deoxy)adenosine, (deoxy)guanosine, (deoxy)cytidine, (deoxy)uridine, (deoxy)thymidine and (deoxy)inosine.
Nucleotide: a nucleoside in which the sugar carries one or more phosphate groups. A nucleotide thus consists of a sugar moiety (pentose), a phosphate group, and a purine or pyrimidine base. Nucleotides are the sub-units of nucleic acids. In the context of the invention, the term “purine nucleotide” signifies a nucleotide in which the base is a purine base. Likewise, a “pyrimidine nucleotide” signifies a nucleotide in which the base is a pyrimidine base. A “guanosine nucleotide” signifies a nucleotide in which the base is guanine, and so on. Nucleotides containing the bases adenine, guanine, cytosine, uracil, thymine are referred to herein using the standard one-letter code A, G, C, U and T respectively. In the context of the invention, and unless otherwise specified, the use of these one-letter codes signifies deoxyribonucleotides, with the exception of U which generally represents a uracil-containing ribonucleotide.
Nucleotide Sequence: a sequence of nucleotides joined together by 3′-5′ phosphodiester bonds to form polynucleotides. According to the invention, nucleotide sequences are represented by formulae whose left to right orientation is in the conventional direction of 5′-terminus to 3′-terminus, unless otherwise specified.
Nucleotide analogue: a purine or pyrimidine nucleotide that differs structurally from one of the Adenosine (A)-, Thymidine (T)-, Guanosine (G)-, Cytidine (C)-, or Uridine (U)-containing nucleotides, but is sufficiently similar to substitute for one of these unaltered nucleotides in a nucleic acid molecule. In the context of the invention, the substitution of a nucleotide by an analogue gives rise to a change in the secondary properties of the nucleic acid, such as stability, bioavailability, solubility, transfectability, induction of side-effects etc, without modifying the primary property of cytotoxicity. The term “nucleotide analogue” encompasses altered bases, different or unusual sugars (i.e. sugars other than the “usual” pentose), altered phosphate backbones, or any combination of these alterations. A listing of exemplary analogues wherein the base has been altered is provided in Table A below:

TABLE A
Nucleotide Analogues

| Abbreviation | Description |
|---|---|
| ac4c | 4-acetylcytidine |
| chm5u | 5-(carboxyhydroxylmethyl)uridine |
| cm | 2′-O-methylcytidine |
| cmnm5s2u | 5-carboxymethylaminomethyl thiouridine |
| d | dihydrouridine |
| fm | 2′-O-methylpseudouridine |
| galq | β,D-galactosylqueosine |
| gm | 2′-O-methylguanosine |
| I | Inosine |
| i6a | N6-isopentenyladenosine |
| m1a | 1-methyladenosine |
| m1f | 1-methylpseudouridine |
| m1g | 1-methylguanosine |
| ml1 | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| mam5u | 5-methylaminomethyluridine |
| mam5s2u | 5-methoxyaminomethyl-2-thiouridine |
| manq | β,D-mannosylmethyluridine |
| mcm5s2u | 5-methoxycarbonylmethyluridine |
| mo5u | 5-methoxyuridine |
| ms2i6a | 2-methylthio-N6-isopentenyladenosine |
| ms2t6a | N-((9-β-ribofuranosyl-2-methylthiopurine- |
| | 6-yl)carbamoyl)threonine |
| mt6a | N-((9-β-ribofuranosylpurine-6-yl)N-methyl- |
| | carbamoyl)threonine |
| mv | uridine-5-oxyacetic acid methylester |
| o5u | uridine-5-oxyacetic acid (v) |
| osyw | wybutoxosine |
| p | pseudouridine |
| q | Queosine |
| s2c | 2-thiocytidine |
| s2t | 5-methyl-2-thiouridine |
| s2u | 2-thiouridine |
| s4u | 4-thiouridine |
| t | 5-methyluridine |
| t6a | N-((9-β-D-ribofuranosylpurine-6- |
| | yl)carbamoyl)threonine |
| tm | 2′-O-methyl-5-methyluridine |
| um | 2′-O-methyluridine |
| yw | wybutosine |
| x | 3-(3-amino-3-carboxypropyl)uridine, (acp3)u |
| araU | β,D-arabinosyl |
| araT | β,D-arabinosyl |

G-rich region: The guanosine-rich region (or “G-rich region”) of the oligonucleotides of the invention is the stretch of nucleotides which constitutes the 5′ extremity of the oligonucleotide, having a minimum length of 6 nucleotides and a maximum length of 9 nucleotides. At least 50% of the nucleotides in the G-rich region are guanosine nucleotides. This region is not composed exclusively of guanosine nucleotides. It contains a purine tract comprising at least 4 consecutive purine nucleotides, within which there is a triple G motif (G-G-G) or “G-triplet”. The 5′ extremity of the triple G motif is separated from the 5′ extremity of the oligonucleotide by, at most, three nucleotides. In other words, the first nucleotide of the G triplet in a 5′ to 3′ direction, is situated at position 1, 2, 3 or 4 of the oligonucleotide. The nucleotide defining the 3′ extremity of the G-rich region is always a guanosine nucleotide. The G-rich region may contain pyrimidine nucleotides, provided that the total number of pyrimidine nucleotides does not exceed 2. When the G-rich region contains 2 pyrimidine nucleotides, they are not consecutive to each other. For the purposes of the invention, the length of the G-rich region is the length of the shortest stretch of nucleotides which simultaneously meets the triple requirement of:
   having a length of 6 to 9 nucleotides,
   having at least 50% guanosine nucleotides.
   ending at the 3′ extremity in a guanosine nucleotide.
Quiescent: a quiescent cell is a cell which is metabolically active but not undergoing either proliferation or death. This state corresponds to the G₀ phase of the cell cycle. Growth and replication stops. Most of the cells in the adult body remain in a quiescent, non-proliferating state, which corresponds to G₀ in the cell cycle. Examples of normally quiescent cell populations in the body are neurons and muscle cells. Cells in G₀ may re-enter the G1 phase of the cell-cycle in response to particular signals, or may die. In vitro, depending on the cell-type, quiescence can be induced by serum starvation, or by contact-inhibition once the cells have reached a certain degree of confluence.
Non-quiescent: a cell which is non-quiescent is in one of the active phases of the cell cycle (G₁, S, G₂ or M) i.e. a cell which is in a state of growth and division. Proliferating cells are thus non-quiescent. In an adult organism, some cell populations such as intestinal epithelial cells and dermal cells are normally proliferating. These populations are however subject to stringent growth control mechanisms. Cancer is an abnormal state in which uncontrolled proliferation of one or more cell populations interferes with normal biological functioning. Cancer cells are therefore also examples of cells which are usually non-quiescent in vivo. The in vitro correlate of cancer is called cellular transformation, exemplified by transformed cell-lines such as transformed human embryonic kidney cells (HEK 293). Such cells are normally proliferative in vitro unless specific measures are taken to arrest growth, such as serum starvation or contact inhibition.
Cell death: Cell death can occur in either a programmed manner (for example apoptosis) or in a non-programmed manner (for example necrosis). Cell death induced by the oligonucleotides of the invention is death having at least one characteristic of programmed cell death, with or without associated necrosis. Programmed cell death: programmed cell death is an active, orderly, and cell-type specific death. As a result of genetic reprogramming of the cell in response to a series of endogenous cell-type-specific signals, biochemical and morphological changes occur within the cell, resulting in its death and elimination. In addition, a variety of exogenous cell damaging treatments (e.g., radiation, chemicals and viruses) can activate this pathway if sufficient injury to the cell occurs. Characteristics of programmed cell death include mitochondrial depolarization, activation of caspases, and positive staining with Annexin V. Unless otherwise specified, the terms “programmed cell death” or “cell death” in the context of the invention signifies cell death having at least one characteristic of programmed cell death, such as mitochondrial depolarization, activation of caspases, or positive staining with Annexin V, with or without associated necrosis. Programmed cell death induced by cytotoxic oligonucleotides of the invention is mediated by mechanisms intrinsic to the cell, not by the suppression of gene products encoded by genes of infectious agents such as viruses or bacteria.
Apoptosis: apoptosis is the principal example of genetically programmed cell death. Apoptosis occurs in response to specific genetically programmed physiological signals, and is characterized by a cellular pattern of chromatin condensation, membrane blebbing (formation of cell membrane-bound vesicles) and single-cell death. Fragmentation of genomic DNA (DNA ladder formation) is the irreversible event that commits the cell to die and occurs before changes in plasma and internal membrane permeability. Visible morphological changes in apoptosis include nuclear chromatin condensation, cytoplasm shrinking, dilation of the endoplasmic reticulum, and membrane blebbing. Dead cells are ingested by neighbouring cells.
Necrosis: Necrotic death can be elicited by any of a large series of nonspecific factors that result in a change in the plasma membrane permeability. This increased plasma membrane permeability results in cellular swelling, organelle disruption and the eventual osmotic lysis of the cell. In necrotic cell death, the cell has a passive role in initiating the process of cell death (i.e. the cell is killed by its hostile microenvironment). Dead cells are ingested by phagocytes. Necrotic cell death can be present in populations of cells undergoing programmed cell death.
Cytotoxic: generally speaking, a cytotoxic substance is one which has a toxic effect on living cells, the cells being thereby injured or killed. In the specific context of the invention, the term “cytotoxic” signifies that the substance in question induces cell death. Unless otherwise specified, cell death induced by a “cytotoxic” substance of the invention is cell death having at least one characteristic element of programmed cell death, with or without accompanying necrosis. According to the invention, oligonucleotides are considered to be cytotoxic (or “active”) when they reproducibly demonstrate significant concentration-dependent cyt6toxicity over the range 0-200 nM in non-confluent vascular endothelial or smooth muscle cells of two different species, whereby a reduction of at least 20% in cell survival, at concentrations of 100 nM compared to mock-transfected controls is considered to represent significant cytotoxicity. Preferably, the reduction in cell. survival is at least 25%, preferably at least 30% and more preferably at least 40% at concentrations of 100 nM. Such a cytotoxicity profile is preferably accompanied by a reduction in cell survival of at least 50% at concentrations of 200 nM, compared to mock-transfected controls.
Cytostatic: a cytostatic substance is one which inhibits or prevents the proliferation and or growth of living cells. A cytostatic substance does not per se induce cell death. Cytostatic agents are also described as “anti-proliferative”.
Higher eukaryotic organism: multicellular eukaryotic organism of the animal or plant kingdom. Preferred organisms are vertebrates, particularly mammals, including humans, and plants.

Turning now more particularly to the cytotoxic G-rich oligonucleotides of the invention, they consist of two contiguous regions, namely:
i) a 5′ G-rich region having 6 to 9 nucleotides, and
ii) a 3′ tail region,

the combined length of the G-rich region and the 3′ tail region being from 20 to 50 nucleotides, particularly 25 to 50 nucleotides.

The 5′ G-rich region of the oligonucleotides of the invention has the formula 1:

Formula 1 Seq. ID No. 86

5′ [X¹-X²-(R¹-R²-R³-R⁴)-X³-X⁴-X⁵-X⁶-X⁷] 3′

wherein:
(R′-R²-R³-R⁴) represents a tract of four consecutive purine nucleotides, each R representing a purine nucleotide,
each of X¹, X², X³, X⁴, X⁵, X⁶ and X⁷ independently represents a nucleotide which may be present or absent, such that the total number of nucleotides in the G-rich region is from 6 to 9,
each of X¹, X² X³, X⁴, X⁵, X⁶ and X⁷ independently represents a purine or pyrimidine nucleotide, such as A, C, T or G,

provided that:
at least 50% of the nucleotides in the G-rich region are guanosine nucleotides,
the portion of the G-rich region represented by X²-(R¹-R²-R³-R⁴) contains a triple guanosine motif (G-G-G),
the portion of the G-rich region represented by X³-X⁴-X⁵-X⁵-X⁶-X⁷ does not contain a thymidine nucleotide downstream of a guanosine nucleotide,
the G-rich region is not composed exclusively of guanosine nucleotides,
the nucleotide defining the 3′ extremity of the G-rich region is a guanosine nucleotide,
the total number of pyrimidine nucleotides in the G-rich region does not exceed 2, and these pyrimidine nucleotides are not consecutive to each other.

In the above Formula 1, each of X¹, X² X³, X⁴, X⁵, X⁶ and X⁷ independently represents a purine or pyrimidine nucleotide, particularly A, C, G or T, provided that the stretch represented by X³-X⁴-X⁵-X⁶-X⁷ does not contain a thymidine nucleotide downstream (i.e. 3′) of a guanosine nucleotide. In other words, the portion of the G-rich region represented by X³-X⁴-X⁵-X⁶-X⁷ is free of GT motifs. Preferably, the whole of the G-rich region is free of GT dinucleotide motifs. In a further embodiment the portion of the G-rich region represented by X³-X⁴-X⁵-X⁶-X⁷ contains no thymidine nucleotides, i.e. each of X³, X⁴, X⁵, X⁶and X⁷ independently represents a guanosine, adenosine or cytosine nucleotide, subject to the conditions imposed by the provisos defined in Formula 1.

When determining the length of the G-rich region of an oligonucleotide according to the invention, the length is the shortest stretch of nucleotides which simultaneously meets the triple requirement of
having a length of 6 to 9 nucleotides,
having at least 50% guanosine nucleotides.
ending at the 3′ extremity in a guanosine nucleotide.

For example, in the case of an oligonucleotide having a 5′ extremity having the sequence 5′-GAGGGGCAG-3′, the G-rich region has 6 nucleotides and consists of the sequence of GAGGGG. This rule for defining the length of the G-rich region applies to each of Formulae 1 to 7 as defined herein.

The 3′ tail region of the oligonucleotides is essentially any nucleotide sequence i.e. there are no stringent sequence requirements for this part of the molecule.

Within the main class of molecules whose G-rich region is defined by Formula 1 above, a number of preferred sub-classes can be distinguished. The G-rich regions of these preferred sub-groups of oligonucleotides are also defined by a series of Formulae 1a to 7, presented below.

According to a first sub-class, oligonucleotides of the invention have a G-rich region having the Formula 1a:

Formula 1a Seq. ID No. 87

5′ [X¹-X²-(R¹-R²-R³-R⁴)-X³-X⁴-X⁵-X⁶-X⁷] 3′

wherein R, X¹, X³, X⁴, X⁵ and X⁶ have the meanings defined in Formula 1, with the additional proviso that if the first 4 nucleotides at the 5′ end of the G-rich region are 4 consecutive guanosine nucleotides, the fifth nucleotide of the G-rich region is a cytosine nucleotide.

According to a second sub-class, oligonucleotides of the invention wherein the purine tract of Formula 1 is immediately flanked by a pyrimidine nucleotide on the 5′ side, have G-rich regions defined by Formula 2:

Formula 2 Seq. ID No. 88

5′ [X¹-Py-(R¹-R²-R³-R⁴)-X³-X⁴-X⁵-X⁶] 3′

wherein R, X², X³, X⁴, X⁵ and X⁶ have the meanings defined in Formula 1, Py represents a pyrimidine nucleotide,
the triple G motif (G-G-G) Is present in the (R¹-R²-R³-R⁴) purine tract,
X¹ is present or absent, and
X³, X⁴, X⁵ and X⁶ may be present or absent such that the total number of nucleotides in the G-rich region is from 6 to 9.

According to a particularly preferred embodiment of the invention, Py in Formula 2 is a cytosine nucleotide. Such embodiments include oligoriucleotides wherein the G-rich region comprises the sequence:

5′ GCGGGG 3′

An example of a cytotoxic oligonucleotide of the invention having this type of G-rich region is:

| | |
|---|---|
| Oligo 100 | GCGGGGACAGGCTAGCTACAACGACAGCTGCAT |

Alternatively, oligonucleotides of the invention wherein the purine tract of Formula 1 is immediately flanked by a pyrimidine nucleotide on the 3′ side, and wherein X¹ and X² in Formula 1 above are both absent, have G-rich regions defined by Formula 3:

Formula 3 Seq. ID No. 89

5′ [(R¹-R²-R³-R⁴)-C-X⁴-X⁵-X⁶-X⁷] 3′

wherein R, X⁴, X⁵, X⁶ and X⁷ have the meanings defined in Formula 1,
the triple G motif (G-G-G) is present in the (R¹-R²-R³-R⁴) purine tract and
X³, X⁴ X⁵, X⁶ and X⁷ may be present or absent such that the total number of nucleotides in the G-rich region is from 6 to 9.

An example of an oligonucleotide of the invention having a G-rich region according to Formula 3 is one in which the G-rich region has the sequence

5′ GGGGCAG 3′,

for example the following oligonucleotide:

| | |
|---|---|
| Oligo 26 | GGGGCAGGAAGCAACATCGATCGGGACTTTTGA. |

According to another sub-class of the invention, the purine tract defined in Formula 1 above is flanked on at least one side by a further purine nucleotide, thereby creating a tract of at least 5 consecutive purine nucleotides. A first example of oligonucleotides of the invention having a purine tract of at least 5 nucleotides are those in which the 5′ G-rich region has the Formula 4:

Formula 4 Seq. ID No. 90

5′ [X¹-(R⁵-R¹-R²-R³-R⁴)-X³-X⁴-X⁵-X⁶] 3′

wherein:
R, X¹, X³, X⁴, X⁵ and X⁶ have the meanings defined in Formula 1,
R⁵ is a purine nucleotide,
(R⁵-R¹-R²-R³-R⁴) represents a tract of five consecutive purine nucleotides,
the triple G motif (G-G-G) is present in the (R⁵-R¹-R²-R³-R⁴) purine tract, and
X¹, X³, X^{4,} X⁵ and X⁶ may be present or absent such that the total number of nucleotides in the G-rich region is from 6 to 9.

An example of a sub-class of oligonucleotides of the invention having a purine tract of at least 5 nucleotides according to Formula 4, are those in which the 5′ G-rich region has the Formula 5:

Formula 5 Seq. ID. No. 91

5′ [X¹-(R⁵-R¹-R²-R³-R⁴)-X³-X⁴-X⁵] 3′

wherein:
R, X¹, X³, X⁴ and X⁵ have the meanings defined in Formula 1,
X¹ is present,
(R⁵-R¹-R²-R³-R⁴) represents a tract of five consecutive purine nucleotides,
the triple G motif (G-G-G) is present in the (R⁵-R¹-R²-R³-R⁴) purine tract, and
X³, X⁴ and X⁵ may be present or absent such that the total number of nucleotides in the G-rich region is from 6 to 9.

A third example of oligonucleotides of the invention having a purine tract of at least 5 nucleotides are those in which the 5′ G-rich region has the Formula 6:

Formula 6 Seq. ID. No. 92

5′ [(R⁵-R¹-R²-R³-R⁴)-X³-X⁴-X⁵-X⁶] 3′

wherein:
R, X³, X⁴, X⁵ and X⁶ have the meanings defined in Formula 1,
(R⁵-R¹-R²-R³-R⁴) represents a tract of five consecutive purine nucleotides,
the triple G motif (G-G-G) is present in the (R⁵-R¹-R²-R³-R⁴) purine tract, and
X³, X⁴, X⁵ and X⁶ may be present or absent such that the total number of nucleotides in the G-rich region is from 6 to 9.

Within the preferred sub-classes of molecules having G-rich regions defined by Formulae 4, 5 and 6, there are further preferred groupings according to whether the (R⁵-R¹-R²-R³-R⁴) purine tract is adenosine-containing or not.

More specifically, a preferred group of oligonucleotides having G-rich regions according to Formula 5 are those wherein the (R⁵-R¹-R²-R³-R⁴) purine tract is adenosine-containing, the G-rich region thereby having the formula 5.1

Formula 5.1 Seq. ID. No. 93
5′ [X¹-(R⁵-R¹-R²-R³-R⁴)-X³-X⁴-X⁵] 3′

wherein at least one of R⁵, R¹, R³ and R⁴ represents A,
X¹ is present and represents a purine or pyrimidine nucleotide (A, C, T or G) and
X³, X⁴, and X⁵ have the meanings defined in Formula 1, and may be present or absent such that the total number of nucleotides in the G-rich region is from 6 to 9.

A particularly preferred sub-group of oligonucleotides having a G-rich region in accordance with Formula 5.1 are those wherein at least one of R⁵ and R¹ is an adenosine nucleotide, and the G-rich region has the formula 5.1a:

Formula 5.1a Seq. ID. No. 94

5′ [X¹-(R⁵-R¹-R²-R³-R⁴)-X³-X⁴-X⁵] 3′

wherein (R⁵-R¹-R²-R³-R⁴) again represents a tract of five consecutive purine nucleotides containing a triple guanosine (G-G-G) motif, at least one of R⁵ and R¹ represents A, X¹ represents a purine or pyrimidine nucleotide, and X³, X⁴, and X⁵ have the meanings defined in Formula 1, and may be present or absent such that the total number of nucleotides in the G-rich region is from 6 to 9.

Another preferred sub-group of oligonucleotides having a G-rich region in accordance with Formula 5.1 are those wherein at least one of R³ and R⁴ is an adenosine nucleotide, and X¹ is any nucleotide other than G. According to this variant of the invention, the G-rich region has the formula 5.1b

Formula 5.1b Seq. ID. No. 95

5′ [X¹-(R⁵-R¹-R²-R³-R⁴)-X³-X⁴-X⁵] 3′

wherein (R⁵-R¹-R²-R³-R⁴) again represents a tract of five consecutive purine nucleotides containing a triple guanosine (G-G-G) motif, at least one of R³ and R⁴ represents A, X¹ represents A, C or T and X³, X⁴, and X⁵ have the meanings defined in Formula 1, and may be present or absent such that the total number of nucleotides in the G-rich region is from 6 to 9.

Typical examples of molecules having G-rich regions according to Formula 5.1 are those wherein the 5′ G-rich region has 6 nucleotides and is chosen from the group consisting of:

| | |
|---|---|
| 5′ [X¹-(AGGGG)] 3′ | Formula 5.1.1 Seq. ID. No. 96 |
| 5′ [X¹-(GAGGG)] 3′ | Formula 5.1.2 Seq. ID. No. 97 |
| 5′ [X¹-(GGGAG)] 3′ | Formula 5.1.3 Seq. ID. No. 98 |

wherein A represents an adenosine nucleotide, and G represents a guanosine nucleotide, and X¹ represents a purine or pyrimidine nucleotide, e.g. A, C, T or G.

Further examples of molecules according to Formula 5.1 are those wherein the 5′ G-rich region has 7 to 9 nucleotides and is chosen from the group consisting of:

Formula 5.1.4 Seq. ID. No. 99

5′ [X¹-(GGGGA)-X³-X⁴-X⁵] 3′

Formula 5.1.5 Seq. ID. No. 100

5′ [X¹-(AGGGA)-X³-X⁴-X⁵] 3′

wherein A represents adenosine and G represents guanosine,

X¹ represents a purine or pyrimidine nucleotide e.g. A, C, T or G,

X³, X⁴, and X⁵ have the meanings defined in Formula 1, and

X⁴ and X⁵ may be present or absent such that the total number of nucleotides In the G-rich region is 7, 8 or 9.

The nucleotide X³ in any one of Formulae 5.1.4 or 5.1.5 may be chosen from any of A, C, G or T. If X³ represents A, C or T, the G-rich region has 8 or 9 nucleotides.

Preferred variants of oligonucleotides having G-rich regions according-to Formula 5.1.3 and Formula 5.1.4 are those wherein X¹ represents any nucleotide other than G. According to these variants, the 5′ G-rich region is chosen from the group consisting of:

Formula (5.1.3b) Seq. ID. No. 101

5′ [X¹-(GGGAG)] 3′

Formula (5.1.4b) Seq. ID. No. 102

5′ [X¹-(GGGGA)-X³-X⁴-X⁵] 3′

wherein A represents an adenosine nucleotide and G represents a guanosine nucleotide, X¹ represents A, C or T, X³, X⁴, and X⁵ have the meanings defined in Formula 1, and X⁴ and X⁵ may be present or absent such that the total number of nucleotides in the G-rich region is 7, 8 or 9.

The nucleotide X¹ in any one of Formulae 5.1.1, 5.1.2, 5.1.3, 5.1.4 or 5.1.5 above may typically be T or C. In such cases, the 5′ G-rich region preferably has one of the following sequences

5′ TGAGGG 3′,
5′ CGGGAG 3′,
5′ TAGGGG 3′.

Alternatively, the nucleotide X¹ in any one of Formulae 5.1.1, 5.1.2, 5.1.3, 5.1.4 or 5.1.5 above, may represent A or G. In such cases, preferred examples of 5′ G-rich regions are those having the sequence

5′ GAGGGG 3′

Specific examples of cytotoxic oligonucleotides having G-rich regions according to Formula 5.1.2 are the following:

| | |
|---|---|
| Oligo 1 | TGAGGGGCAGGCTAGCTACAACGACGTCGTGA(3′-3′C) |
| Oligo 2 | TGAGGGGCAAGCAACATCGATCGGCGTCGTGA(3′-3′C) |
| Oligo 3 | TGAGGGGCAGGCTAGCTACAACGACGTCGCGG(3′-3′G) |
| Oligo 14 | (5′-5′T)GAGGGGCAGGCTAGCTACAACGACGTCGTGAC |
| Oligo 15 | (5′-5′T)GAGGGGCAGGCTAGCTACAACGACGTCGTGA |
| | (3′-3′C) |
| Oligo 16 | TGAGGGGCAGGCTAGCTACAACGACGTCGTGAC |
| Oligo 17 | TGAGGGGCAGGCTAGCTACAACGACGTCGTGAC |
| Oligo 18 | TGAGGGGCAGGCTAGCTACAACGACGTCGTGAC |
| Oligo 25 | TGAGGGGCAGGCTAGCTACAACGACGTCGTGAC |
| Oligo 66 | TGAGGGGCAGN₂₅ |
| Oligo 67 | TGAGGGGCN₂₇ |
| Oligo 68 | TGAGGGN₂₉ |

wherein each N independently represents G, T, C or A, and may be the same or different, and (3′-3′) and (5′-5′) signifies an inverted 3′ or 5′ linkage respectively.

Specific examples of cytotoxic oligonucleotides having G-rich regions according to Formula 5.1.3 are the following:

| | |
|---|---|
| Oligo 4 | CGGGAGGAAGGCTAGCTACAACGAGAGGCGTTG(3′-3′T) |
| Oligo 9 | CₘGGGAGGAAGGCTAGCTACAACₘGAGA |
| | GGCₘGTTG(3′-3′T) |
| Oligo 10 | CGGGAGGAAGGCTAGCTACAAGCAGAGGGCTTG |
| | (3′-3′T) |
| Oligo 11 | CGGGAGGAAGGCTAGCTACAACGAGAGGCGTTG-X |
| Oligo 12 | CGGGAGGAAG(N₂₀) |
| Oligo 13 | CGGGAGGAAG(N₂₅) |
| Oligo 19 | CGGGAGGAAGGCTAGCTACAACGAGAGGCGTTG |
| Oligo 20 | CGGGAGGAAGGCTAGCTACAACGAGAGGCGTUG |
| | (3′-3′T) |
| Oligo 27 | CGGGAGGAAGGCTAGCTACAACAAGAGGCGTTG |
| | (3′-3′T) |
| Oligo 28 | CGGGAGGAAAGCAACATCGATCGG(3′-3′T) |
| Oligo 29 | CGGGAGGAAGGCTAGCACACAGAGGGTCATGGT |
| | (3′-3′T) |
| Oligo 30 | CGGGAGGAAG(N₂₃)[3′-3′T] |
| Oligo 31 | (5′P)CGGGAGGAAGGCTAGCTACAACGAGAG |
| | GCGTTG |
| Oligo 43 | CGGGAGGAAG(N₁₅) |
| Oligo 63 | CGGGAGGAN₂₇ |
| Oligo 64 | CGGGAGN₂₉ |
| Oligo 70 | CGGGAGGAAG(TAG)₈ |
| Oligo 80 | AGGGAGGGAGGAAGGGAGGGAGGGAGGG |
| Oligo 81 | AGGGAGGGAGGAAGGGAGGGAGGGAGGGAGGG |
| Oligo 83 | CGGGAGGAAGGCTAGCTACAACGAGAGGCGTTG-B |

wherein each N independently represents G, T, C or A, and may be the same or different, X represents cholesteryl-TEG, (5′P) represents a 5′ phosphorylation, Cₘ represents a methylated cytosine, B represents biotin, and (3′-3′) and (5′-5′) signifies an inverted 3′ or 5′ linkage respectively.

Specific examples of cytotoxic oligonucleotides having G-rich regions according to Formula 5.1.1 are the following:

| | |
|---|---|
| Oligo 101 | GAGGGGGAAGGCTAGCTACAACGAAGTTCGTCC |
| Oligo 24 | GAGGGGCAGGCTAGCTACAACGACGTCGTGA |

A further preferred group of oligonucleotides having G-rich regions according to Formula 5 are those wherein the (R⁵-R¹-R²-R³-R⁴) purine tract is devoid of adenosine nucleotides and the G-rich region has the formula 5.2:

| | |
|---|---|
| 5′ [X¹-(G-G-G-G-G)] 3′ | Formula 5.2 Seq. ID. No. 103 |

wherein X¹ represents A, C or T.

Another preferred group of oligonucleotides are those having G-rich regions according to Formula 6, wherein the (R⁵-R¹-R²-R³-R⁴) purine tract is adenosine-containing. These molecules have G-rich regions having the formula 6.1

Formula 6.1 Seq. ID. No. 104

5′ [(R⁵-R¹-R²-R³-R⁴)-X³-X⁴-X⁵-X⁶] 3′

wherein at least one of R⁵, R¹, R³ and R⁴ represents A,

X³, X⁴, X⁵ and X⁶ have the meanings defined in Formula 1 and may be present or absent such that the total number of nucleotides in the G-rich region is from 7 to 9.

Typical examples of molecules having G-rich regions according to Formula 6.1 are those wherein the 5′ G-rich region is chosen from the group consisting of:

Formula 6.1.1 Seq. ID. No. 105

5′ [(AGGGG)-X³-X⁴-X⁵-X⁶] 3′

Formula 6.1.2 Seq. ID. No. 106

5′ [(GAGGG)-X³-X⁴-X⁵-X⁶] 3′

Formula 6.1.3 Seq. ID. No. 107

5′ [(GGGAG)-X³-X⁴-X⁵-X⁶] 3′

Formula 6.1.4 Seq. ID. No. 108

5′ [(GGGGA)-X³-X⁴-X⁵-X⁶] 3′

Formula 6.1.5 Seq. ID. No. 109

5′ [(AGGGA)-X³-X⁴-X⁵-X⁶] 3′

wherein A represents adenosine and G represents guanosine, and
X³, X⁴, X⁵ and X⁶ have the meanings defined in Formula 1, and
X⁴, X⁵ and X⁶ may be present or absent such that the total number of nucleotides in the G-rich region is from 6 to 9.

The nucleotide X³ in any one of Formulae 6.1.1, 6.1.2, 6.1.3, 6.1.4, 6.1.5 may typically be A or C and the G-rich region has 7, 8 or 9 nucleotides. An example of such a G-rich region is:

5′ AGGGGCAG 3′

Alternatively, the nucleotide X³ in any one of Formulae 6.1.1, 6.1.2, 6.1.3, 6.1.4 or 6.1.5 may be G, and the G-rich region thus has 6 nucleotides. Examples of such 5′ G-rich regions include:

5′ GGGAGG 3′
5′ AGGGAG 3′
5′ AGGGGG 3′

The nucleotide X³ in any one of Formulae 6.1.4 or 6.1.5 may be T, and the G-rich region has 7, 8 or 9 nucleotides.

Specific examples of cytotoxic oligonucleotides having G-rich regions according to Formula 6.1.3 are the following:

| | |
|---|---|
| Oligo 8 | GGGAGGAAGGCTAGCTACAACGAGAGGCGTT(3′-3′T) |
| Oligo 72 | GGGAGGAAAGN₂₅ |
| Oligo 73 | GGGAGGAAAGN₂₀ |
| Oligo 74 | GGGAGGAAAGN₁₅ |

where each N independently represents G, T, C or A, and may be the same or different, and (3′-3′) signifies an inverted 3′ linkage.

Specific examples of cytotoxic oligonucleotides having G-rich regions according to Formula 6.1.5 are the following:

| | |
|---|---|
| Oligo 80 | AGGGAGGGAGGAAGGGAGGGAGGGAGGG |
| Oligo 81 | AGGGAGGGAGGAAGGGAGGGAGGGAGGGAGGG |

A specific example of a cytotoxic oligonucleotide having a G-rich region according to Formula 6.1.1 is the following:

| | |
|---|---|
| Oligo 23 | AGGGGCAGGCTAGCTACAACGACGTCGTG |

Yet another preferred group of oligonucleotides are those having G-rich regions according to Formula 6, wherein the (R⁵-R¹-R²-R³-R⁴) purine tract is devoid of adenosine nucleotides and the G-rich region has the formula 6.2:

Formula 6.2 Seq. ID. No. 110

5′ [(G-G-G-G-G)-X³-X⁴-X⁵-X⁶] 3′

wherein X³ represents A or C, and

X⁴, X⁵ and X⁶ have the meanings defined in Formula 1 and may be present or absent such that the total number of nucleotides in the G-rich region is from 7 to 9.

According to a further variant of the invention, the oligonucleotide capable of inducing cell death in non-quiescent eukaryotic cells, is an oligonucleotide consisting exclusively of purine nucleotides. According to this variant, the oligonucleotide has a length of 20 to 50 nucleotides, for example 25 to 50 nucleotides, and consists of
iii) a 5′ G-rich region having 6 to 9 nucleotides, and
iv) a 3′ tail region,
   wherein the 5′ G-rich region has the formula 7:

Formula 7 Seq. ID. No. 111

5′ [R⁶-R⁵-(R¹-R²-R³-R⁴)-R⁷-R⁸-R⁹-R¹⁰-R¹¹] 3′

in which
each R represents a purine nucleotide,
(R¹-R²-R³-R⁴) represents a tract of four consecutive purine nucleotides,
each of R⁵, R⁶, R⁷, R⁸, R⁹, R¹⁰ and R¹¹ independently represents a purine nucleotide which may be present or absent, such that the total number of nucleotides in the G-rich region is from 6 to 9,
   provided that:
at least 50% of the nucleotides in the G-rich region are guanosine nucleotides,
the portion of the G-rich region represented by R⁵-(R¹-R²-R³-R⁴) contains a triple guanosine motif (G-G-G),
the G-rich region is not composed exclusively of guanosine nucleotides,
the nucleotide defining the 3′ extremity of the G-rich region is a guanosine nucleotide,
and the 3′ tail region consists of purine nucleotides.

Examples of all-purine oligonucleotides according to this embodiment of the invention are those having G-rich regions chosen from the group consisting of:

Formula 7.1 Seq. ID. No. 112

5′ [R⁶-(AGGGG)] 3′

Formula 7.2 Seq. ID. No. 113

5′ [R⁶-(GAGGG)] 3′

Formula 7.3 Seq. ID. No. 114

5′ [R⁶-(GGGAG)] 3′

Formula 7.4 Seq. ID. No. 115

5′ [R⁶-(GGGGA)-R⁷-R⁸-R⁹] 3′

Formula 7.5 Seq. ID. No. 116

5′ [R⁶-(AGGGA)-R⁷-R⁸-R⁹] 3′

Formula 7.6 Seq. ID. No. 117

5′ [(AGGGG)-R⁷-R⁸-R⁹-R¹⁰] 3′

Formula 7.7 Seq. ID. No. 118

5′ [(GAGGG)-R⁷-R⁸-R⁹-R¹⁰] 3′

Formula 7.8 Seq. ID. No. 119

5′ [(GGGAG)-R⁷-R⁸-R⁹-R¹⁰] 3′

Formula 7.9 Seq. ID. No. 120

5′ [(GGGGA)-R⁷-R⁸-R⁹-R¹⁰] 3′

Formula 7.10 Seq. ID. No. 121

5′ [(AGGGA)-R⁷-R⁸-R⁹-R¹⁰] 3′

wherein each of R⁶, R⁷, R⁸, R⁹, R¹⁰ independently represent a purine nucleotide, and may be present or absent such that the total number of nucleotides in the G-rich region is from 6 to 9.

The all-purine oligonucleotides of the invention have a length of from 20 to 50 nucleotides, particularly 21 to 50 nucleotides. Molecules of this type having lengths as short as 20, 21, 22, 23 or 24 nucleotides have been shown to have efficacy in inducing cell-death, having characteristics of programmed cell death. Particularly preferred lengths of all-purine cytotoxic oligonucleotides of the invention are 21 to 50 nucleotides, for example 22 to 48 nucleotides, or 24 to 45 nucleotides, or 25 to 40 nucleotides.

Specific examples of active all-purine oligonucleotides of the invention include the following

| | |
|---|---|
| Oligo 78 | AGGGAGGGAGGAAGGGAGGG |
| Oligo 79 | AGGGAGGGAGGAAGGGAGGGAGGG |
| Oligo 80 | AGGGAGGGAGGAAGGGAGGGAGGGAGGG |
| Oligo 81 | AGGGAGGGAGGAAGGGAGGGAGGGAGGGAGGG |
| Oligo 82 | (AGGG)₆ |

It can thus be seen from the above that cytotoxic oligonucleotides of the invention can have G-rich regions corresponding to any one of the Formulae 1, 2, 3, 4, 5, 6 and 7 or to any of the above-defined sub-groups of these Formulae, covalently linked to a 3′ tail region, giving rise to a molecule having a total length of 20 to 50 nucleotides, or 25 to 50 nucleotides.

The 3′-tail region of the oligonucleotides can be substantially any nucleotide sequence. Indeed, there are no rigid sequence requirements for this part of the molecule, as shown by the conservation of cytotoxic activity even after randomization of tail sequences. It is however preferred that the 3′-tail region be composed of a mixture of at least 2 different nucleotides, preferably a mixture of purine and pyrimidine nucleotides, and most preferably a mixture of the four principal nucleotides A, C, T and G.

According to a preferred embodiment, the 3′ tail region is generated randomly from an equimolar mix of A, C, T and G nucleotides. This gives rise to a pool of oligonucleotides. The pool has cytotoxic activity according to the invention, and can be used as such for inducing cell death, or can be further purified to isolate individual cytotoxic oligonucleotides. The invention thus also encompasses pools or mixtures of oligonucleotides wherein at least one oligonucleotide within the mixture has a G-rich region according to any one of Formulae 1 to 7, and has cytotoxic activity as defined herein.

Alternatively, the 3′ tail region may contain only purine nucleotides. In this case, it is preferred that the 3′ tail be a mixture of A's and G's rather than exclusively A's or G's.

Typical oligonucleotides of the invention are those consisting of a G-rich region according to any one of Formula 1 to 7 as defined above, covalently linked to a 3′ tail containing at least two different nucleotides, preferably at least 3 different nucleotides including G, and most preferably four different nucleotides generated randomly. Tails consisting of a single nucleotide such as polyA tails, or homoG polymers are not preferred.

Whilst the oligonucleotides of the invention are single stranded, it has nevertheless been observed by the inventors that 3′ tail regions containing two sequences capable of together forming a hairpin structure within the tail, do not have reduced cytotoxicity. Thus a region of double strandedness within the 3′ tail of the oligonucleotide may be tolerated. However, it is preferred that the tail region of the oligonucleotide be devoid of sequences capable of forming a hairpin structure with sequences within the G-rich region, as such hairpin formation may have a detrimental effect on the cytotoxicity of the oligonucleotide.

According to a further embodiment of the invention, the cytotoxic oligonucleotides are devoid of sequences defining ribozyme or DNAzyme catalytic regions, for example sequences defining functional ribozyme or DNAzyme catalytic regions. Indeed, the inventors have demonstrated that the cytotoxic activity of G-rich oligonucleotides herein which comprise functional DNAzyme catalytic regions, does not correlate with their catalytic activity. Cytotoxic activity of the oligonucleotides of the invention is thus independent of their catalytic activity. In other words, this invention demonstrates the cytotoxic acitivity is related to the Formulae, as disclosed in this invention, and this activity is separate and distinct from a determination of whether or not the oligonucleotides have catalytic activity. Examples of such a further class of oligonucleotides of the invention are those which do not contain the DNAzyme catalytic region having the sequence 5′-GGCTAGCTACMCGA-3′ or its reverse sequence 5′-AGCMCATCGATCGG-3′, or variants of these sequences having one or two base substitutions or deletions. In particular, according to this embodiment, the cytotoxic oligonucleotides are free of the sequence 5′-GGCTANCTACMCGA-3′, where N represents a guanosine or a cytosine nucleotide, or its reverse sequence 5′-AGCAACATCNATCGG-3′. According to this variant, the oligonucleotides of the invention thus consist of a G-rich region according to any one of Formulae 1 to 7 as defined above, and a 3′ tail region, the oligonucleotide being devoid of the sequence 5′-GGCTANCTACAACGA-3′, or its reverse sequence. As an example, this category of oligonucleotides of the invention may have a G-rich region according to any one of Formulae 1 to 7 as defined above, and a 3′ tail region which does not comprise the sequence GGCTAGCTACMCGA, or its reverse sequence.

For example, in accordance with this variant of the invention, the 3′ tail region of the oligonucleotide does not comprise:
the sequence GGCTAGCTACMCGAGAGGCGTT, or
the sequence GGCTAGCTACMCGACGTTGC, or
the sequence GGCTAGCTACAACGACAGCTGCAT.

Further examples of oligonucleotides according to this embodiment, are those wherein the 3′ tail region of the oligonucleotide does not consist of:
the sequence GAAGGCTAGCTACAACGMGTTCGTCC(3′-3′T), or
the sequence TCAGGCTAGCTACAACGACGTGGTGGT, or
the sequence GAAGGCTACCTACAACGAGAGGCGTTG(3′-3′T), or
the sequence GCAGGCTAGCTACAACGACGTCGTGA(C) wherein (C) represents a 3′ terminal cytosine which may or may not be modified by a 3′-terminal inversion, or
the sequence GCTAGCTACAACGACGTCG(C), wherein (C) represents a 3′ terminal cytosine which may or may not be modified by a 3′-terminal inversion, or
the sequence CAGGCTAGCTACAACGACGTCGC(G), wherein (G) represents a 3′ terminal guanosine which may or may not be modified by a 3′-terminal inversion, or
the sequence GCAGGCTAGCTACMCGACGTCGCG(G), wherein (G) represents a 3′ terminal guanosine which may or may not be modified by a 3′-terminal inversion, or
the sequence GCMGCMCATCGATCGGCGTCGTGA(3′-3′C).

Specific examples of oligonucleotides of the invention which are free of DNAzyme catalytic regions such as 5′-GGCTANCTACAACGA-3′ and its reverse sequence as defined above include the following:

| | |
|---|---|
| Oligo 12 | CGGGAGGAAG(N₂₀) |
| Oligo 13 | CGGGAGGAAG(N₂₅) |
| Oligo 27 | CGGGAGGAAGGCTAGCTACAACAAGAGGCGTTG(3′-3′T) |
| Oligo 29 | CGGGAGGAAGGCTAGCACACAGAGGGTCATGGT(3′-3′T) |
| Oligo 30 | CGGGAGGAAG(N₂₃)[3′-3′T] |
| Oligo 43 | CGGGAGGAAG(N₁₅) |
| Oligo 63 | CGGGAGGA(N₂₇) |
| Oligo 64 | CGGGAG(N₂₉) |
| Oligo 65 | CGGG(N₃₁) |
| Oligo 66 | TGAGGGGCAG(N₂₅) |
| Oligo 67 | TGAGGGGC(N₂₇) |
| Oligo 68 | TGAGGG(N₂₉) |
| Oligo 70 | CGGGAGGAAG(TAG)₈ |
| Oligo 72 | GGGAGGAAAG(N₂₅) |
| Oligo 73 | GGGAGGAAAG(N₂₀) |
| Oligo 74 | GGGAGGAAAG(N₁₅) |
| Oligo 78 | AGGGAGGGAGGAAGGGAGGG |
| Oligo 79 | AGGGAGGGAGGAAGGGAGGGAGGG |
| Oligo 80 | AGGGAGGGAGGAAGGGAGGGAGGGAGGG |
| Oligo 81 | AGGGAGGGAGGAAGGGAGGGAGGGAGGGAGGG |
| Oligo 82 | (AGGG)₆ |

According to another embodiment of the invention, the oligonucleotides of the invention may contain sequences defining DNAzyme catalytic regions, for example the GGCTAGCTACAACGA sequence referred to above, particularly when the G-rich regions of the oligonucleotide have a sequence corresponding to any one of Formulae 5.1.4, 5.1.5, 5.2 and 6.2 as herein defined.

The oligonucleotides of the invention have a length of 25 to 50 nucleotides, for example 26 to 45 nucleotides. Particularly preferred oligoucleotides have a length of 30 to 44 nucleotides, for example 31 to 42 nucleotides.

The oligonucleotides of the invention are active in a chemically unmodified form. However various substitutions by analogues and chemical derivatives of nucleotides can be made to improve characteristics such as stability, bioavailability, solubility, transfection efficiency etc. For example, oligonucleotides having 2′-OH modified nucleotides such as 2′O-methyl, 2′O-alkyl, 2′-methoxyethyl or those with other modified ribose chemistries may have desirable properties. Such modifications can be made throughout the molecule. Further examples of analogues and derivatives are listed in Table A above.

The oligonucleotides of the invention having a native phosphodiester backbone are active. However, activity may be modulated, and secondary properties enhanced, by judicious use of modified backbone chemistries such as phosphoroamidate, phosphorothioate, amide-3, methylenemethylimino, peptide nucleic acid, methyl phosphonate, phosphorodithioate chemistries among others. Extensive modification of the sequence with alternative base, sugar and backbone chemistries may, however, have a deleterious effect on the biological activity. In particular, total replacement of the phosphodiester backbone with phosphorothioate linkages greatly reduces the activity of these oligonucleotide sequences. Partial replacement is therefore preferred.

Chemical modifications that protect the termini of the oligonucleotides from exonucleases are particularly beneficial. These include but are not limited to the use of 3′-3′ and 5′-5′ linked nucleotides (inverted linkages).

The oligonucleotides may also be substituted using groups such as cholesterol, biotin, dyes with linkers etc. These substitutions are made at the 3′ end of the molecule, so as not to adversely affect cytotoxicity.

Thus, according to a preferred embodiment, an oligonucleotide of the invention, consisting of a 5′ G-rich region according to any one of Formulae 1 to 7 as defined above, and a 3′ tail region, can be chemically modified such that it comprises one or more of the following:
at least one nucleotide which is modified at the 2′-OH position, for example substitution with a 2′-O-methyl, particularly in nucleotides at the 5′ and 3′ extremities, or
at least one methylated cytosine, particularly a cytosine in a GC dinucleotide sequence, or
a partially modified phosphodiester backbone, for example from one to nine phosphorothioate linkages at the 5′ and/or 3′ end, or
a 5′ phosphorylation, or
a 3′-3′ and/or 5′-5′ inverted linkage(s), or
a 3′ terminal substitution by groups such as cholesterol, biotin, dyes, markers etc.

Typical examples of modified oligonucleotides according to the invention are those derived from Oligo 4 by any one or more of the above modifications. Examples include Oligo 9 (methylated cytosines), Oligo 11 (cholesterol substitution), Oligo 83 (3′ biotinylated), Oligo 20 (2′-O-methyl substitutions) and Oligo 31 (5′ phosphorylation). Other examples include molecules derived from Oligo 1 by any one or more of the above modifications, for example Oligos 16, 17 and 18 (phosphorothioate linkages), and Oligos 14 and 15 (inverted 5′-5′ and/or 3′-3′ linkages).

With regard to the biological effect of the oligonucleotides of the invention, they specifically induce cell death in non-quiescent eukaryotic cells when they are introduced into the cells. The induced cell death has features characteristic of programmed cell death, including caspase activation, phospholipid phosphatidylserine translocation and mitochondrial depolarisation. According to the invention, within a population of cells, whilst the majority of cells undergo cell death having features of the programmed-type, cell death by necrosis may also occur in a minority of cells. In the context of the invention, the programmed cell death induced by the cytotoxic oligonucleotides is mediated by mechanisms intrinsic to the cell, not by the suppression of genes of infectious agents such as viruses or bacteria, or the products of such genes, for example LMP1 encoded by EBV etc. The cytotoxic effect of the oligonucleotides of the invention can therefore be obtained in cells and cell-lines which are not infected by infectious agents.

The cytotoxic effect according to the invention is non-species specific i.e., following appropriate transfection, an oligonucleotide of the invention induces cell death in cell lines originating from different species, for example from human or rodent species. Moreoever, within a given species, the cytotoxic effect is seen in cells of different tissue or neoplastic origin for example in vascular endothelial cells, smooth muscle cells, embryonic kidney cells, cervical cancer cell lines etc. The cytotoxic effect is thus not tissue-specific in any given species.

Within a population of cells, the cell death obtained according to the invention may be accompanied by inhibition of cell-cycling, proliferation and migration, and by reductions in the secretion of cytokines.

According to the invention, the oligonucleotides produce the cytotoxic effect with marked potency in actively proliferating and/or migrating cells, but show no significant cytotoxic effect on quiescent cells, particularly in contact-inhibited quiescent cells. Cell death induced by the oligonucleotides of the invention is thus specific for proliferating and/or migrating cells.

The design rules elaborated in the context of the present invention and summarised in Formulae 1 to 7, define groups of oligonucleotides, the vast majority of which have the desired cytotoxic effect. Once the rules have been used to design an oligonucleotide having a length of 20 to 50 nucleotides, for example 21 to 50, preferably 25 to 50 nucleotides, and a 5′ G-rich region according to the invention, the oligonucleotide is tested for its capacity to induce cell death by carrying out the following cytotoxicity assay (a), together with at least one of additional tests (b) to (e):
a) a cytotoxicity assay (or cell viability assay) is carried out on cells transfected with the oligonucleotide under test to determine the proportion of cells surviving, 48 hours after transfection, in comparison with a mock-transfected control. The assay system distinguishes between viable and non-viable cells, for example by exploiting the ability of viable cells to convert a redox dye such as resazurin into a fluorescent compound such as resorufin. The cytotoxicity assay is carried out on proliferating, non-confluent cells of two different animal species, for example human and rat endothelial or smooth muscle cells, in order to ascertain the non-species specificity of the induced cell death. The oligonucleotides are generally used at concentrations ranging from 0 to 400 nM, particularly 0 to 200 nM. Oligonucleotides are considered to be cytotoxic (or “active”) when they reproducibly demonstrate significant concentration-dependent cytotoxicity over the range 0-200 nM in non-confluent vascular endothelial or smooth muscle cells of two different species. A reduction of at least 20% cell survival, preferably at least 25%, more preferably at least 30% and most preferably at least 40% at concentrations of 100 nM compared to mock-transfected controls is considered to represent significant cytotoxicity. The reduction in cell survival of at least 20% at 100 nM is preferably accompanied by a reduction of at least 50% of cell survival at concentrations of 200 nM.
b) microscopic signs of cell death are assessed in cells transfected with the oligonucleotide under test, 24 and 48 hours after treatment at concentrations sufficient to induce cell death (approximately 25 to 200 nM). These signs include shrinking and detachment of cells and formation of cell debris;
c) assessment of mitochondrial deDolarization is carried out in vascular endothelial cell-lines transfected with the oligonucleotide under test at a concentration sufficient to induce significant cell death (50-200 nM). Cells are trypsinized and harvested 12-48 hours later, making sure that detached cells are also collected. The cells are then incubated with an appropriate marker of mitochondrial potential, for example JC-1, and analysed in a Fluorescence Activated Cell Sorter (FACS) to determine the frequency of cells demonstrating green-shifted fluorescence. An active oligonucleotide as described in this invention may, at concentrations capable of inducing cytotoxicity, cause significant depolarization of mitochondria relative to untreated or mock-transfected vascular endothelial cells.
d) detection of activation of caspases: vascular endothelial or smooth muscle cells are transfected with 50-200 nM of the relevant oligonucleotide. Cells are harvested and contacted with a suitable marker of caspase activation such as a fluorogenic caspase substrate. Approximately 2 hours later, the cells are analyzed by flow cytometry or fluorescence microscopy. Polyclonal and monoclonal antibodies for detecting caspase activation by Western blot, immunoprecipitation, and immunohistochemistry may also be used. Active oligonucleotides of the invention may have the ability to induce significant caspase activation above and beyond the baseline caspase activity in the endothelial or smooth muscle cells.
e) staining of cells with Annexin V: active oligonucleotides of the invention may cause the exposure of the inner membrane phosphatidyl serines in vascular endothelial cells. Harvested cells having undergone transfection with the relevant oligonucleotide are stained with labeled recombinant annexin V, as a marker of programmed cell death. The cells can be simultaneously stained with propidium iodide, as a marker of membrane permeabilization, so that the various populations of cells with characteristic staining can be estimated.

Positivity of staining with Annexin V is considered characteristic of programmed forms of death such as apoptosis and autophagy. Cells that are positive only for propidium iodide staining are understood to be undergoing necrotic cell death. Active oligonucleotides according to the invention may give rise to positive staining with Annexin V, and additionally may also be positive for propidium iodide staining.

Preferably, in determining whether oligonucleotides fitting the design rules are cytotoxic according to the invention, the cytotoxicity assay (a) is carried out in association with at least one of the tests (b) to (e), for example tests (b), (d) and (e). Oligonucleotides which give positive results in test (a) and in one of tests (b) to (e) are considered to be active in the context of the invention. Whilst a variety of cells or cell lines can be used for the above tests, it is preferred that human microvascular endothelial cells (HMEC-1 cells) and rat smooth muscle cells (RSMCs) be used.

Full details of the activity tests (a) to (e) are presented in Example 5 in the Experimental Section below.

The precise mechanism by which the ODNs of the invention bring about cell death is not yet fully elucidated. However, it is postulated that the oligonucleotides are recognised by an intracellular protein, triggering the engagement of cell death programmes. In particular, the inventors have demonstrated the binding of the oligonucleotides of the invention to eukaryotic elongation factor 1 alpha 1 (eEFA1,A1, formerly designated EF1alpha1). This “moonlighting” protein is, amongst other things, a major sensor of growth-related signals and an apoptotic regulator in times of endoplasmic reticulum stress. It is a key factor in protein synthesis, where it promotes the transfer of aminoacylated tRNAs to the A site of the ribosome (Ejiri S. I. 2002). The induction of cell death in non-quiescent cells by the oligonucleotides of the invention is thus possibly related to their ability to bind to eEF1A-1.

Furthermore, it has been demonstrated that the mechanism underlying the cytotoxic effect of the oligonucleotides of the invention is unrelated to “CpG” effects. Indeed, methylation of CpG motifs contained within the oligonucleotides of the invention does not affect cytotoxic potency compared to the unmethylated version of the same molecule, and inversion of the CpG motif to GpC also has no effect on capacity of the molecules to induce cell death.

It has also been demonstrated that the cytotoxic effect of the oligonucleotides of the invention is apparently not brought about by known or hypothesized RNA targeting mechanisms. According to the invention, cytotoxicity is maintained after scrambling (or randomizing) of the 3′ region of the oligonucleotides. As a result of scrambling, any complementarity which the oligonucleotide might have had towards a cellular target molecule, is destroyed, and yet the cytotoxicity is conserved. Consequently, the mechanism underlying the activity of the oligonucleotides of the invention appears to be distinct from that underlying antisense, ribozyme, DNAzyme, RNAi effects.

The oligonucleotide-induced cell death according to the invention Is thus characterised by:
i) the conservation of the cytotoxic properties of the oligonucleotide after scrambling of the 3′ tail region and/or,
ii) the ability of the oligonucleotide to bind to eukaryotic elongation factor 1 alpha 1 (eEF1A1).

It can therefore be determined whether an oligonucleotide is inducing cell death by the present invention by testing one or both of the above parameters.

According to the invention, the cytotoxic oligonucleotides induce cell death having features of programmed cell death, in a variety of different types of cells and cell lines of higher eukaryotic organisms, particularly mammalian cells such as human, mouse, rat, pig, horse, dog, monkey, cat, rabbit cells etc. With regard to tissue origin of the cells which are sensitive to the oligonucleotides of the invention, it has been found that vascular endothelial and smooth muscle cells, fibroblasts, retinal epithelium and embryonic kidney cells are particularly sensitive. Cell death can be induced according to the invention in both primary cells and established cell lines. Moreover, a variety of cell types of neoplastic origin are susceptible to the cytotoxic oligonucleotides, for example human cervical carcinoma cell lines, lung carcinoma etc.

The invention also relates to a method of inducing, in a population of non-quiescent eukaryotic cells, cell death having at least one characteristic of programmed cell death, the method comprising contacting cells of said population in vitro, in vivo or ex vivo with at least one G-rich oligonucleotide, said oligonucleotide consisting of two contiguous regions, namely:
i) a 5′ G-rich region corresponding to any one of Formulae 1 to 7 as defined above, and
ii) a 3′ tail region,
   the combined length of the G-rich region and the 3′ tail region being from 20 to 50 nucleotides, particularly 25 to 50 nucleotides. Optionally the method may further comprise a step of detecting cell death having at least one characteristic of programmed cell death in at least a portion of the population of cells. Such detection step may be carried out in vitro, in vivo or ex vivo. The provisos listed above in connection with the G-rich region of Formulae 1 to 7 also apply to oligonucleotides used in the in vivo, in vitro or ex vivo method of the invention.

According to this method, the G-rich oligonucleotides are used in an amount sufficient to induce cell death in at least a portion of the population of cells containing said oligonucleotide. Preferably, at least 20%, for example at least 25%, and preferably at least 40%, of the cells in the population undergo cell death having features of programmed cell death, within 24 to 48 hours of the introduction of the oligonucleotide(s).

When the method is carried out in vitro, the cells may be primary cells or established cell lines, and may be of mammalian, for example human origin. They are used in conditions in which the cells proliferate. Such in vitro methods are useful for screening cytotoxic oligonucleotides of the invention for example with a view to selecting oligonucleotides having optimized properties as a result of sequence variations, chemical modifications, inclusion of analogues, substituents etc. The in vitro method of the invention may also provide a diagnostic method, for example for the detection of proliferating cells, or for the selection of quiescent cells. The method may also be an ex-vivo method.

When the method is carried out in vivo the cell population is a population within a higher eukaryotic organism, for example a mammal, particularly a human. Such in vivo methods include therapeutic and/or prophylactic methods in the context of diseases involving aberrant proliferation of cells. In vivo methods may also include in vivo screening of cytotoxic oligonucleotides of the invention with a view to selecting oligonucleotides having optimized cytotoxic activity, stability, absence of side-effects etc. In such methods, the higher eukaryotic organism may or may not be suffering from a disorder involving aberrant proliferation of cells.

Thus, according to the invention, the cytotoxic oligonucleotides having G-rich regions of any one of Formulae 1 to 7 as defined above are used in methods of treatment or prevention of disorders involving aberrant cell proliferation and/or migration. They are also used in the manufacture of medicaments for the treatment or prevention of such disorders.

Specifically, this aspect of the invention relates to a method of treating or preventing a disorder involving aberrant cell proliferation, comprising administering to a patient in need of such treatment a cytotoxic oligonucleotide of the invention to induce cell death in abnormally proliferating cells and to treat or prevent the disorder.

More particularly, in a first aspect, the invention relates to a method of inducing, in non-quiescent eukaryotic cells, cell death having at least one characteristic of programmed cell death for treating or preventing a disorder involving abnormal cell proliferation or migration comprising administering to a subject in need of such treatment a pharmaceutically effective amount of an oligonucleotide, wherein said oligonucleotide has a length of 25 to 50 nucleotides and consists of:
i) a 5′ G-rich region having 6 to 9 nucleotides, and
ii) a 3′ tail region,
   wherein the 5′ G-rich region has the formula 1:

Formula 1

5′ [X¹-X²-(R¹-R²-R³-R⁴)-X³-X⁴-X⁵-X⁶-X⁷] 3′

in which
(R¹-R²-R³-R⁴) represents a tract of four consecutive purine nucleotides, each R representing a purine nucleotide,
each of X¹, X², X³, X⁴, X⁵, X⁶ and X⁷ independently represents a nucleotide which may be present or absent, such that the total number of nucleotides in the G-rich region is from 6 to 9,
each of X¹, X² X³, X⁴, X⁵, X⁶ and X⁷ independently represents a purine or pyrimidine nucleotide,
   provided that:
at least 50% of the nucleotides in the G-rich region are guanosine nucleotides,
the portion of the G-rich region represented by X²-(R¹-R²-R³-R⁴) contains a triple guanosine motif (G-G-G),
the portion of the G-rich region represented by X³-X⁴-X⁵-X⁶-X⁷ does not contain a thymidine nucleotide downstream of a guanosine nucleotide,
the G-rich region is not composed exclusively of guanosine nucleotides,
the nucleotide defining the 3′ extremity of the G-rich region is a guanosine nucleotide,
the total number of pyrimidine nucleotides in the G-rich region does not exceed 2, and these pyrimidine nucleotides are not consecutive to each other, and the 3′ tail region is any nucleotide sequence.

Examples of compounds which are useful in this first method of treatment or prevention are compounds having G-rich regions which meet any of the following formulae as defined herein: Formula 1, Formula 2, Formula 3, Formula 4, Formula 5, Formula 5.1, Formula (5.1.1); Formula (5.1.2); Formula (5.1.3), Formula (5.1.4); Formula (5.1.5); Formula 5.2; Formula 6; Formula 6.1; Formula (6.1.1); Formula.(6.1.2); Formula (6.1.3); Formula (6.1.4); Formula (6.1.5) or Formula 6.2, the provisos and definitions of the 3′ tail region as listed above for the first aspect also applying to these compounds.

In a second aspect, the invention relates to another method of inducing, in non-quiescent eukaryotic cells, cell death having at least one characteristic of programmed cell death for treating or preventing a disorder involving abnormal cell proliferation or migration comprising administering to a subject in need of such treatment a pharmaceutically effective amount of an oligonucleotide, wherein said oligonucleotide has a length of 20 to 50 nucleotides and consists of
i) a 5′ G-rich region having 6 to 9 nucleotides, and
ii) a 3′ tail region,
   wherein the 5′ G-rich region has the formula 7:

Formula 7

5′ [R⁶-R⁵-(R¹-R²-R³-R⁴)-R⁷-R⁸-R⁹-R¹⁰-R¹¹] 3′

in which
each R represents a purine nucleotide,
(R¹-R²-R³-R⁴) represents a tract of four consecutive purine nucleotides, each of R⁵, R⁶, R⁷, R⁸, R⁹, R¹⁰ and R¹¹ independently represents a purine nucleotide which may be present or absent, such that the total number of nucleotides in the G-rich region is from 6 to 9,
   provided that:
at least 50% of the nucleotides in the G-rich region are guanosine nucleotides,
the portion of the G-rich region represented by R⁵-(R¹-R²-R³-R⁴) contains a triple guanosine motif (G-G-G),
the G-rich region is not composed exclusively of guanosine nucleotides,
the nucleotide defining the 3′ extremity of the G-rich region is a guanosine nucleotide,
   and the 3′ tail region consists of purine nucleotides.

Examples of compounds which are useful in this second method of treatment or prevention are compounds having G-rich regions which meet any of the following formulae as defined herein: Formula 7, Formula 7.1, Formula 7.2, Formula 7.3, Formula 7.4, Formula 7.5, Formula 7.6, Formula 7.7, Formula 7.8, Formula 7.9, Formula 7.10, the provisos and definitions of the 3′ tail region as listed above for the second aspect also applying to these compounds.

In a third aspect, the invention relates to yet another method of inducing, in non-quiescent eukaryotic cells, cell death having at least one characteristic of programmed cell death for treating or preventing a disorder involving abnormal cell proliferation or migration comprising administering to a subject in need of such treatment a pharmaceutically effective amount of an oligonucleotide, said oligonucleotide having a length of 25 to 50 nucleotides and consisting of
i) a 5′ G-rich region having from 6 to 9 nucleotides, and
ii) a 3′ tail region,
   wherein the 5′ G-rich region has the formula 1a:

Formula 1a

5′ [X¹-X²-(R¹-R²-R³-R⁴)-X³-X⁴-X⁵-X⁶-X⁷] 3′

in which

(R¹-R²-R³-R⁴) represents a tract of four consecutive purine nucleotides, each R representing a purine nucleotide,

each of X¹, X², X³, X⁴, X⁵, X⁶ and X⁷ independently represents a nucleotide which may be present or absent, such that the total number of nucleotides in the G-rich region is from 6 to 9,

each of X¹, X² X³, X⁴, X⁵, X⁶ and X⁷ independently represents a purine or pyrimidine nucleotide,

provided that:
at least 50% of the nucleotides in the G-rich region are guanosine nucleotides,
the portion of the G-rich region represented by X²-(R¹-R²-R³-R⁴) contains a triple guanosine motif (G-G-G),
the portion of the G-rich region represented by X³-X⁴-X⁵-X⁶-X⁷ does not contain a thymidine nucleotide downstream of a guanosine nucleotide,
the nucleotide defining the 3′ extremity of the G-rich region is a guanosine nucleotide,
the total number of pyrimidine nucleotides in the G-rich region does not exceed 2, and these pyrimidine nucleotides are not consecutive to each other,
if the first 4 nucleotides at the 5′ end of the G-rich region are 4 consecutive guanosine nucleotides, the fifth nucleotide of the G-rich region is a cytosine nucleotide,
   and the 3′ tail region is any nucleotide sequence,

provided the oligonucleotide does not contain the sequence 5′-GGCTANCTACMCGA-3′, or its inverse sequence 5′-AGCAACATCNATCGG-3′ wherein N represents a guanosine or cytosine nucleotide.

Examples of compounds which are useful in this third method of treatment or prevention are compounds having G-rich regions which meet any of the following formulae as defined herein: Formula 2, Formula 3, Formula 5.1a, Formula 5.1b, Formula (5.1.1), Formula (5.1.2), Formula (5.1.5), Formula (5.1.3b), Formula (5.1.4b), Formula 5.2, Formula 6.1, Formula (6.1.1), Formula (6.1.2), Formula (6.1.3), Formula (6.1.5), the provisos and definitions of the 3′ tail region as listed above for the third aspect also applying to these compounds.

The conditions in which the oligonucleotide is administered to the subject (for example the dose, schedule, mode of administration etc) are in pharmaceutically acceptable amounts such that cell death having at least one characteristic of programmed cell death, is obtained in the abnormally proliferating cells. In general, the oligonucleotides of the invention are administered at doses ranging from 0.1 to 100 mg per kilo of patient body weight, for example 0.1 to 50 mg/kg. Systemic administration may require doses in the upper part of said range, for example 5 to 100 mg/kg, whereas routes of administration directly at the site of the lesion may require lower doses such as 0.1 to 10 mg/kg. The dose is preferably such as to achieve a concentration of active agent at the site of action of 1 to 200 nM.

Cell death induced by the oligonucleotides of the invention has been shown to be specific for proliferating cells. Consequently, it is envisaged that treatment according to the invention will be free from harmful side effects arising from non-specific cell death. Cell death, having characteristics of programmed cell death, is induced in at least a part of the abnormally proliferating cellular population.

The oligonucleotides of the present invention can be administered in a variety of dosage forms adapted to the chosen route of administration. Thus, the oligonucleotides can be administered, orally or parenterally, intravenously, intra-arterially, intramuscularly, topically, subcutaneously, intradermally, vaginally, rectally, or nasally or as an inhalation. Additional routes of administration include intraocular, intravitreal, juxtascleral, subretinal, intraconjunctival, intra-articular, intra-lesional, intra-vesicular, intraportal, intraperitoneal or intrathecal routes. The oligonucleotides can be systemically administered by infusion or injection. Solutions of the oligonucleotides can be prepared in sterile water that can be mixed with a nontoxic surfactant. Dispersions can be prepared in glycerol liquid polyethylene glycols, and oils. Drug-eluting solid forms may also be used. The preparations may contain a preservative to prevent the growth of microorganisms.

Formulations for oral administration can be presented in the form of capsules, cachets, or tablets each containing a pharmaceutically acceptable amount of the oligonucleotide of the present invention. They can also be in the form of powder or granules, as a solution or suspension in an aqueous or non-aqueous liquid or an oil-in-water liquid emulsion or a water-in-oil liquid emulsion. The oligonucleotide may be presented as a bolus, electuary or paste.

Tablets may be made by compression or molding. One or more accessory ingredients such as binders, lubricants, diluents, preservatives, disintegrants, surface-active or dispersing agents may be added. The tablets may be compressed using a suitable machine. Molded tablets can be made by molding In a suitable machine a mixture of the powdered oligonucleotide moistened with an inert diluent. The tablets may further be optionally coated or formulated with hydroxypropylmethyl cellulose in varying proportions to provide a sustained release tablet.

In another aspect the oligonucleotides of the present invention can be formulated in the form of lozenges for oral application. The lozenges may contain a flavoring, as well as the oligonucleotides of the present invention in a pharmaceutically acceptable amount. Pastilles comprising the oligonucleotide in an inert vehicle such as gelatin and glycerin are also contemplated by the present invention.

The liquid formulation may contain inert diluents commonly used in the art such as water and other drinkable solvents. This formulation may contain solubilizing agents and emulsifiers such as ethyl alcohol, isopropyl alcohol, propylene glycol, various oils and glycerol.

Vaginal or rectal formulations can be prepared, for example, using cocoa butter, polyethylene glycol, a suppository wax or a salicylate. They can be delivered as a suppository and therefore are solid at room temperature, but liquid at body temperature and therefore melt in the rectum or vaginal cavity.

According to the Invention, the patient to be treated is a human or animal subject.

Moreover, because the oligonucleotides of the invention induce cell death rather than simply exerting a cytostatic effect, beneficial effects going beyond disease stabilization are to be expected. Such effects include cell shrinkage and regression of lesions and neoformations resulting from aberrant proliferation, for example tumour regression and vessel regression in cases of unwanted neovascularization.

In another embodiment the present invention relates to a method for shrinking cells and regressing lesions, said method comprising administering to a patient in need of such treatment a pharmaceutically acceptable amount of the oligonucleotides of the present invention in a pharmaceutically acceptable carrier. In this case the pharmaceutically acceptable amounts are those which cause shrinkage of the cells and regression of lesions.

In accordance with the invention, the in vivo cytotoxic effects of the oligonucleotides may be observed in a number of different forms, and may be tested using a variety of models. For example, in the area of ocular angiogenesis, a widely-recognized model is the laser-induced Choroidal Neovascularization (CNV) in rats, as described in the examples below, or any other suitable model representative of angiogenesis. The oligonucleotides of the invention, when administered after onset of aberrant proliferation, cause significant shrinkage of neoformations and prevent their further development. In a clinical setting, the oligonucleotides give rise to a regression of lesions through cell death which can be detected inter alia by in situ assays for apoptosis (e.g. TUNEL method) and/or caspase activation or other suitable in situ techniques.

Disorders involving aberrant cell proliferation which are treated or prevented in accordance with the present invention include angiogenesis related disorders, cancer, proliferative dermatological and muscle disorders and inflammatory diseases. The highly specific cytotoxic treatments according to the invention are particularly suitable for individuals in whom significant pathological cell proliferation has already taken place.

Angiogenesis related disorders include solid tumors; blood-borne tumors such as leukemias; tumor metastasis; benign tumors, for example hemangiomas, neurofibromas, trachomas; pre-malignant tumors; rheumatoid arthritis; psoriasis; ocular angiogenic diseases, for example, diabetic retinopathy, retinopathy of prematurity, age-related macular degeneration (AMD), corneal graft rejection, neovascular glaucoma myocardial angiogenesis; plaque neovascularization; angiofibroma; restenosis, pre-neoplastic lesions.

In yet another embodiment, the present invention relates to a method of treating angiogenesis related disorders, the method comprising administering to a patient in need of such treatment a pharmaceutically acceptable amount of the cytotoxic oligonucleotides of the present invention in a pharmaceutically acceptable carrier. For ophthalmic applications the oligonucleotide of the present invention can be formulated in a solution or as eye drops or for injection or eye ointments. Conventional additives in this type of formulation include isotonizing agents such as sodium chloride, mannitol and sorbitol, buffers such as phosphate, borate or citrate, pH adjusting agents, preservatives such as paraoxybenzoic acid esters, sorbic acid and chlorhexidine and chelating agents.

Cancers which can be treated using the oligonucleotides of the invention include melanoma, skin, bladder, non-small cell lung, small cell lung, lung, hepatocarcinoma, retinoblastoma, astrocytoma, glioblastoma, neuroblastoma, head, neck, breast, pancreatic, prostate, renal, bone, testicular, ovarian, cervical, gastrointestinal lymphoma, brain, and colon cancer.

In yet another aspect the present invention concerns a method for treating cancer said method comprising administering to a patient in need of such treatment a pharmaceutically acceptable amount of an oligonucleotide of the present invention in a pharmaceutically acceptable carrier. This pharmaceutically acceptable amount may vary depending on the type of cancer one wants to treat.

Proliferative dermatologic disorders include conditions such as keloids, seborrheic keratosis, verruca arising from papilloma virus infection, eczema and psoriasis.

Thus, the present invention also relates to treating or preventing dermatological disorders by topically administering to a patient in need of such treatment a pharmaceutically acceptable amount of an oligonucleotide of the present invention in a pharmaceutically acceptable carrier. In this particular aspect the oligonucleotide can be formulated in a cream, a gel, lotions, ointments, foams, patches, solutions and sprays for topical application. In another aspect the oligonucleotides can be formulated into a skin covering or a dressing containing a pharmaceutically acceptable amount. In another aspect the oligonucleotides of the present invention can be formulated in a controlled release system. The skin coverings or dressing material can be any material used in the art such as bandage, gauze, sterile wrapping, hydrogel, hydrocolloid and similar materials. The oligonucleotide may also be administered via the intra-lesion route.

The ointments, pastes, creams and gels may contain in addition to the pharmaceutically acceptable amount of the oligonucieotide of the present invention, excipients such as animal and vegetable fats, silicones, starch, tragacanth, cellulose derivatives, oils, waxes, parrafins, zinc oxide and talc, or mixtures thereof.

Sprays can contain in addition to the oligonucleotides of the present invention, excipients such -as aluminium hydroxides and calcium silicates, as well as propellants such as chlorofluorohydrocarbons, butane and/or propane.

Inflammatory diseases include rheumatoid arthritis, uveitis and retinitis. In yet another aspect the present invention relates to a method of treating or preventing inflammatory diseases by administering to a patient in need of such treatment a pharmaceutically effective amount of the oligonucleotide of the present invention in a pharmaceutically acceptable carrier.

An oligonucleotide of the invention, which may be wholly synthetic, is administered to an animal or human in a suitable pharmaceutical carrier at an appropriate dose to generate the desired therapeutic effect i.e., the effect of induction of cell death.

The oligonucleotides may also be expressed in the target cells by transfection with a plasmid encoding for the sequence or by transduction with a genetically-engineered virus encoding the sequence.

Acceptable pharmaceutical carriers include aqueous solutions such as, but not limited to: water, saline, buffers, dextrose-saline. Non-aqueous carriers include oils, oil-water emulsions, liposomes, nanoparticulate carriers, cationic lipids, dendrimers, poly-lysine and other poly-cationic macromolecules or polymers. Both aqueous and non-aqueous carriers may include excipients, stabilisers, anti-microbials, bacteriostats, anti-oxidants as well as bulking agents.

Direct conjugation of the oligonucleotide to targeting ligands such as the RGB peptide sequence, folic acid, transferrin and cholesterol is considered for cell-specific delivery of the sequence when direct application of the sequence to the target cell is not practicable. Additionally, conjugation with other moieties such as poly-ethylene glycol, albumin and other carrier polymers and macromolecules may enhance the biopharmaceutical properties of the sequence. In particular, these may assist with preventing non-specific uptake of the sequence by non-target tissues.

The desired sequences can be administered alone or as a combination of several active sequences. Judicious mixing of several active sequences can result in synergistic activity.

As discussed above, the desired route of administration may include the intravenous, sub-cutaneous, inhalation, intramuscular, intradermal, oral, nasal, topical and rectal routes of administration. In addition, specific anatomical sites of injection such as intra-articular, intra-vesicular, intraperitoneal, intraocular, juxtascleral, subretinal, intravitreal, transdermal may also be used to achieve the desired therapeutic effect. In general, the preferred routes of administration for particular medical conditions are exemplified below in Table 3.

The mode of administration will depend on the route of delivery and will include but not be limited to the use of syringe, catheter, suppository, nebulizer, inhaler, particle-gun, transdermal patch, iontophoresis device, implant, stent, cream, ointment, salve, drops, tablet, capsule and powder.

The required dose is commensurate with the mode of administration and the properties of the sequence in relation to administration of the said sequence.

The duration and frequency of treatment will be as required for the generation and maintenance of the desired therapeutic effect.

Treatment with the sequence may take the form of monotherapy or be part of a broader treatment involving other active treatment modalities as required, for example with one or more additional pharmaceutical agents as a combined preparation for separate, simultaneous or sequential use in therapy.

A number of embodiments of the Invention have been described. Nevertheless, it will be understood that various modifications may be made without departing from the spirit and scope of the invention.

TABLE 3

| ADMINISTRATION ROUTE | EXAMPLE OF MEDICAL CONDITION |
|---|---|
| Intraocular, intravitreal, | Neoangiogenic disorders of the back of |
| juxtascleral, subretinal | eye; |
| | Proliferative retinopathy; |
| | Uveitis |
| Intraconjunctival | Corneal neoangiogenesis; |
| | Pterygium |
| Topical | Psoriasis; |
| | Skin cancer (BCC, melanoma, SCC); |
| | Inflammation; |
| | Eczema; |
| | Conjunctivitis; |
| | Corneal angiogenesis |
| Intra-articular | Arthritis |
| Intra-lesional | Solid tumours; |
| | Skin cancer; |
| | Sarcoid |
| Intra-vesicular | Bladder cancer |
| Intravenous, intra-arterial, | Systemic disorders; |
| sub-cutaneous, intramuscular | Solid tumours; |
| | Leukemia |
| Drug-eluting solid forms | Cardiac by-pass stent; |
| | CNS tumours (eg glioblastoma); |
| | Orthopaedic conditions treated by implant |
| Intraportal/intrarterial | Cirrhosis; |
| | Liver cancer; |
| | Metastatic cancer; |
| | Biliary duct cancer |
| Intraperitoneal | Ovarian cancer; |
| | Peritoneal disease; |
| | Pelvic disease |
| Intrathecal | CNS (spinal cord) conditions |
| Colonic irrigation/ | GIT inflammatory diseases eg Crohn's |
| administration | Disease |
| Aerosol | Asthma; |
| | Pulmonary fibrosis |

### Examples

### 1. Cytotoxicity Studies

### Example 1.1

### Cytotoxicity Assay in HMEC-1 Cell Line

The SV-40 transformed human dermal microvascular endothelial cell line (HMEC-1) was maintained in MCDB131 medium containing 10% fetal bovine serum (FBS), 2 mM L-glutamine, 10 ng/mL epidermal growth factor, 1 μg/mL hydrocortisone and 5 U/mL penicillin-streptomycin. The SV-40 transformed rat smooth muscle cells (RSMC) were grown in Waymouth's medium containing 10% FBS, 2-mM L-glutami ne and 5 U/mL penicillin-streptomycin. Cytotoxicity assays were performed as follows: cells were seeded at 5000 cells per well in 96-well black microclear plates (Greiner). After 24 hours, HMEC-1 cells in growth medium containing 5% FBS or RSMCs in growth medium containing 10% FBS were transfected with different concentrations of ODNs in triplicates using FuGENE6 (Roche). FuGENE6: DNA ratio of 3:1 (μL FuGENE6/μg DNA) was used for all transfections. FuGENE6 reagent alone was used as the mock transfection control. Complexation was routinely performed at an ODN concentration of 2 μM and the DNA complex was then serially diluted two-fold prior to a further 10× dilution upon addition to cells. Cell survival was assessed 48 hours post-transfection using a fluorometric cell viability assay for viable cell dehydrogenase activity (CellTiter™-Blue Cell Viability Assay; Promega). Media in wells were replaced with 100 μL OptiMEM to which 20 μL of the assay mix was added. After 2 hours at 37° C., fluorescence was measured at 544_{Ex}/590_{Em} using FLUOstar OPTIMA (BMG Labtechnologies).

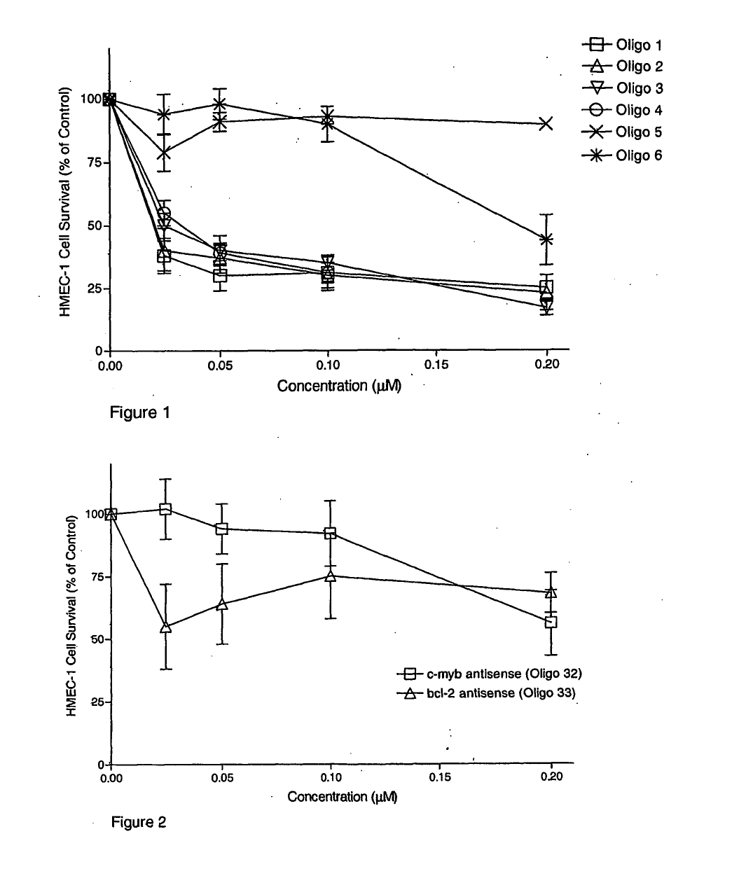

FIG. 1 shows cytotoxicity of 6 ODNs tested in HMEC-1 cells. Similar dose-response curves were obtained for the RSMCs (data not shown). The 4 active ones have a common feature in that they contain purine-rich 5′-ends that include either a G quartet or triplet. The 4 active ODN sequences are aligned below:

| | |
|---|---|
| Oligo 1: | TGAGGGGCAGGCTAGCTACAACGACGTCGTGA(3′-3′C) |
| Oligo 2: | TGAGGGGCAAGCAACATCGATCGGCGTCGTGA(3′-3′C) |
| Oligo 3: | TGAGGGGCAGGCTAGCTACAACGACGTCGCGG(3′-3′G) |
| Oligo 4: | CGGGAGGAAGGCTAGCTACAACGAGAGGCGTTG(3′-3′T) |

A comprehensive list of oligonucleotides that induced HMEC-1 (human microvascular endothelial cell) cell death at less than or equal to 100 nM is shown in Table 1. Oligonucleotides which were ineffective (at ≦100 nM) are presented in Table 2. Representative dose response curves for oligonucleotides from Tables 1 and 2 are shown in the Figures.

### Example 1.2

### Comparison with Oligonucleotides Reported to have Pleiotropic Effects on HMEC-1 Cells

Fully phosphorothioated antisense molecules reported to have “pleiotropic”, non-antisense-mediated effects due to CpG or polyG motifs were tested against HMEC-1 cells. These include the antisense to c-myb, GTGCCGGGGTCTTCGGGC (Oligo 32) (Anfossi et al, 1989) and the antisense to bcl-2, G3139, TCTCCCAGCGTGCGCCAT (Oligo 33) (Cotter et al., 1994). Both molecules were inactive (FIG. 2).

Additional oligonucleotides with 5′ G-rich sequences that have been reported to act by non-antisense mechanisms were investigated using the same procedures.

The nucleolin binding GRO29A (Oligo 84) was without activity over the same concentration range. Likewise, Oligo 87, a topoisomerase I binding aptamer was without activity. Oligo 86, a 36mer ATM-inducing oligonucleotide (nur-E-karnal, JBC 278:12475-12481, 2003) was active, but only appreciably so in the HMEC-1 cells and not the RSMC. Investigation of ATM function in response to Oligo 4 in HMEC-1 cells showed a lack of induction of p53 and no increased phosphorylation of NBS-1 (an ATM substrate). Furthermore, the cytotoxicity of Oligo 4 was not inhibited by Wortmannin, an inhibitor of ATM. This indicates that Oligo 86 acts on cells by a mechanism different to that of the class of oligonucleotides according to the present invention.

TABLE 1
OLIGONUCLEOTIDES SHOWING CYTOTOXIC ACTIVITY

| | |
|---|---|
| N° | |
| SEQ ID | FIG. |

| | | | | |
|---|---|---|---|---|
| 5 | Oligo 1 | TGAGGGGCAGGCTAGCTACAACGACGTCGTGA(3′-3′C) | Inverted 3′ linkage | 1, 11, 20, 23, |
| | | | | 24 |
| 14 | Oilgo 2 | TGAGGGGCAAGCAACATCGATCGGCGTCGTGA(3′-3′C) | Inverted 3′ linkage | 1 |
| 6 | Oligo 3 | TGAGGGGCAGGCTAGCTACAACGACGTCGCGG(3′-3′G) | Inverted 3′ linkage | 1 |
| 20 | Oligo 4 | CGGGAGGAAGGCTAGCTACAACGAGAGGCGTTG(3′-3′T) | Inverted 3′ linkage | 1, 3, 5-7, 9, |
| | | | | 12-17, 19, 27, |
| | | | | 29, 30 |
| 30 | Oligo 8 | GGGAGGAAGGCTAGCTACAACGAGAGGCGTT(3′-3′T) | Oligo 4 minus 1 | 6 |
| | | | base at each end | |
| 26 | Oligo 9 | CGGGAGGAAGGCTAGCTACAACGAGAGGCGTTG(3′-3′T) | Oligo 4 with | 4 |
| | | | methylated | |
| | | | cytosines at CpGs | |
| 23 | Oligo 10 | CGGGAGGAAGGCTAGCTACAAGCAGAGGGCTTG(3′-3′T) | CpGs of oligo 4 | 4 |
| | | | changed to GpCs | |
| 28 | Oligo 11 | CGGGAGGAAGGCTAGCTACAACGAGAGGCGTTG-X | Oligo 4 with X = | 13 |
| | | | cholesterylTEG | |
| 39 | Oligo 12 | CGGGAGGAAG(N₂₀) | N = A, C, G or T | 8 |
| 40 | Oligo 13 | CGGGAGGAAG(N₂₅) | N = A, C, G or T | 8 |
| 2 | Oligo 14 | (5′-5′T)GAGGGGCAGGCTAGCTACAACGACGTCGTGAC | Oligo 1 with 5′-5′T | 11 |
| 3 | Oligo 15 | (5′-5′T)GAGGGGCAGGCTAGCTACAACGACGTCGTGA(3′- | Oligo 1 with 5′-5′T | 11 |
| | | 3′C) | and 3′-3′C | |
| 9 | Oligo 16 | TGAGGGGCAGGCTAGCTACAACGACGTCGTGAC | Oligo 1 with 9 + 9 | 10 |
| | | | phosphorothioate | |
| | | | linkages (PS) | |
| 10 | Oligo 17 | TGAGGGGCAGGCTAGCTACAACGACGTCGTGAC | Oligo 1 with 7 + 7 | 10 |
| | | | phosphorothioate | |
| | | | linkages (PS) | |
| 11 | Oligo 18 | TGAGGGGCAGGCTAGCTACAACGACGTCGTGAC | Oligo 1 with 5 + 5 | 10 |
| | | | phosphorothioate | |
| | | | linkages (PS) | |
| 25 | Oligo 19 | CGGGAGGAAGGCTAGCTACAACGAGAGGCGTTG | Unmodified Oligo 4 | 12 |
| 27 | Oligo 20 | CGGGAGGAAGGCTAGCTACAACGAGAGGCGTUG(3′-3′T) | Oligo 4 with 2 × 2′- | 12 |
| | | | OMethyl at both | |
| | | | ends | |
| 1 | Oligo 23 | AGGGGCAGGCTAGCTACAACGACGTCGTG | | 20 |
| 4 | Oligo 24 | GAGGGGCAGGCTAGCTACAACGACGTCGTGA | | 20 |
| 13 | Oligo 25 | TGAGGGGCAGGCTAGCTACAACGACGTCGTGAC | | 20, 28 |
| 67 | Oligo 26 | GGGGCAGGAAGCAACATCGATCGGGACTTTTGA | | 21 |
| 21 | Oligo 27 | CGGGAGGAAGGCTAGCTACAACAAGAGGCGTTG(3′-3′T) | Inverted 3′ linkage | 19, 22 |
| 22 | Oligo 28 | CGGGAGGAAAGCAACATCGATCGG(3′-3′T) | Inverted 3′ linkage | 23 |
| 24 | Oligo 29 | CGGGAGGAAGGCTAGCACACAGAGGGTCATGGT(3′-3′T) | Inverted 3′ linkage | 25 |
| 41 | Oligo 30 | CGGGAGGAAG(N₂₃)[3′-3′T] | N = A, C, G or T | 25 |
| 29 | Oligo 31 | (5′P)CGGGAGGAAGGCTAGCTACAACGAGAGGCGTTG | 5′-phosphorylated oligo 4 | 26 |
| 45 | Oligo 43 | CGGGAGGAAG(N₁₅) | N = A, C, G or T | 8 |
| 31 | Oligo 47 | CGGGAGGAAGGCTACCTACAACGAGAGGCGTTG(3′-3′T) | Inverted 3′ linkage | 19 |
| 47 | Oligo 63 | CGGGAGGA(N₂₇) | N = A, C, G or T | |
| 48 | Oligo 64 | CGGGAG(N₂₉) | N = A, C, G or T | |
| 49 | Oligo 65 | CGGG(N₃₁) | N = A, C, G or T | |
| 16 | Oligo 66 | TGAGGGGCAG(N₂₅) | N = A, C, G or T | 28 |
| 17 | Oligo 67 | TGAGGGGC(N₂₇) | N = A, C, G or T | 28 |
| 18 | Oligo 68 | TGAGGG(N₂₉) | N = A, C, G or T | 28 |
| 42 | Oligo 70 | CGGGAGGAAG(TAG)₈ | | |
| 54 | Oligo 72 | GGGAGGAAAG(N₂₅) | N = A, C, G or T | |
| 55 | Oligo 73 | GGGAGGAAAG(N₂₀) | N = A, C, G or T | |
| 56 | Oligo 74 | GGGAGGAAAG(N₁₅) | N = A, C, G or T | |
| 63 | Oligo 78 | AGGGAGGGAGGAAGGGAGGG | | |
| 59 | Oligo 79 | AGGGAGGGAGGAAGGGAGGGAGGG | | 18 |
| 60 | Oligo 80 | AGGGAGGGAGGAAGGGAGGGAGGGAGGG | | 18 |
| 61 | Oligo 81 | AGGGAGGGAGGAAGGGAGGGAGGGAGGGAGGG | | 18 |
| 62 | Oligo 82 | (AGGG)₆ | | 18 |
| 46 | Oligo 83 | CGGGAGGAAGGCTAGCTACAACGAGAGGCGTTG-B | B = biotin | |
| 66 | Oligo 100 | GCGGGGACAGGCTAGCTACAACGACAGCTGCAT | | |
| 65 | Oligo 101 | GAGGGGGAAGGCTAGCTACAACGAAGTTCGTCC | | |

TABLE 2
Inactive Oligonucleotides:

FIG.

| | | | | |
|---|---|---|---|---|
| 74 | Oligo 5 | CCGCTGCCAGGCTAGCTACAACGACCCGGACGT(3′-3′T) | | 1 |
| 75 | Oligo 6 | GCCAGCCGCGGCTAGCTACAACGATGGCTCCAC(3′-3′T) | | 1 |
| 76 | Oligo 7 | GCGACGTGAGGCTAGCTACAACGAGTGGAGGAG(3′-3′T) | | 14-17, 29, |
| | | | | 30 |
| 77 | Oligo 32 | GTGCCGGGGTCTTCGGGC | c-myb antisense all | 2 |
| | | | PS | |
| 78 | Oligo 33 | TCTCCCAGCGTGCGCCAT | bcl-2 antisense all PS | 2 |
| 79 | Oligo 34 | CTGGAGGAAGGCTAGCTACAACGAGAGGCGTTG(3′-3′T) | Oligo 4 G->T at 2nd | 6 |
| | | | base | |
| 80 | Oligo 35 | GGAGGAAGGCTAGCTACAACGAGAGGCGT(3′-3′T) | Oligo 4 minus 2 bases | 6 |
| | | | at both 5′ & 3′ ends | |
| 81 | Oligo 36 | TTAGGGTTAGGGTTAGGGTTAGGG(3′-3′T) | Telomere motif which | 5 |
| | | | neutralizes CpG effect | |
| 82 | Oligo 37 | TCCTGGCGGGGAAGT(3′-3′T) | CpG inhibitory motif | 5 |
| 35 | Oligo 38 | CXGGAGGAAGGCTAGCTACAACGAGAGGCGTTG(3′-3′T) | X = 7-deaza-dG | 7 |
| 36 | Oligo 39 | CGXGAGGAAGGCTAGCTACAACGAGAGGCGTTG(3′-3′T) | X = 7-deaza-dG | 7 |
| 37 | Oligo 40 | CGGXAGGAAGGCTAGCTACAACGAGAGGCGTTG(3′-3′T) | X = 7-deaza-dG | 7 |
| 43 | Oligo 41 | CGGGAGGAAG(Nx5) | N = A, C, G or T | 8 |
| 44 | Oligo 42 | CGGGAGGAAG(Nx10) | N = A, C, G or T | 8 |
| 12 | Oligo 44 | TGAGGGGCAGGCTAGCTACAACGACGTCGTGAC | Oligo 1 completely PS | 10 |
| 68 | Oligo 45 | ACGGGAGGAAGGCTAGCTACAACGAGAGGCGTTGA(3′-3′T) | Inverted 3′ linkage | |
| 69 | Oligo 46 | GACGGGAGGAAGGCTAGCTACAACGAGAGGCGTTGAG(3′- | Inverted 3′ linkage | |
| | | 3′T) | | |
| 32 | Oligo 48 | TGGGAGCCAGGCTAGCTACAACGAAGCGAGGCT | | 21 |
| 33 | Oligo 50 | CGGGAGGAACGAGGCGTTG(3′-3′T) | Inverted 3′ linkage | 22 |
| 72 | Oligo 51 | CTGGAGGAACGAGGCGTTG(3′-3′T) | Inverted 3′ linkage | 22 |
| 73 | Oligo 52 | CAACGAGAGGCGTTG(3′-3′T) | Inverted 3′ linkage | 22 |
| 53 | Oligo 53 | CGGGAGGAA(3′-3′T) | Inverted 3′ linkage | 23 |
| 34 | Oligo 54 | CGGGAGGAAGGCTAGCTACAACGA(3′-3′T) | Inverted 3′ linkage | 23 |
| 8 | Oligo 55 | TGAGGGGCA(3′-3′T) | Inverted 3′ linkage | 24 |
| 15 | Oligo 56 | TGAGGGGCAAGCAACATCGATCGG(3′-3′T) | Inverted 3′ linkage | 24 |
| 7 | Oligo 57 | TGAGGGGCAGGCTAGCTACAACGA(3′-3′T) | Inverted 3′ linkage | 24 |
| 50 | Oligo 58 | CGGGAGGAAG(A₂₃)[3′-3′T] | PolyA Tail | 25 |
| 70 | Oligo 59 | TTGGAGGGGGTGGTGGGG | G rich oligo | 26 |
| 51 | Oligo 60 | CGGGAGGAAG(R₂₅) | R = A, C or T | 26 |
| 38 | Oligo 61 | CAACGCCTCTCGTTGTAGCTAGCCTTCCTCCCG | | 27 |
| 19 | Oligo 69 | TGAG(N₃₁) | N = A, C, G or T | 28 |
| 52 | Oligo 71 | CGGGAGGAAG(TAGGAT)₄ | | |
| 57 | Oligo 75 | GGGAGGAAAG(N₁₀) | N = A, C, G or T | |
| 58 | Oligo 76 | GGGAGGAAAG(N₅) | N = A, C, G or T | |
| 64 | Oligo 77 | AGGGAGGGAGGAAGGG | | 18 |
| 71 | Oligo 84 | TTTGGTGGTGGTGGTTGTGGTGGTGGTGG | | |
| 83 | Oligo 85 | TGTTTGTTTGTTTGTTTGTTTGTTTGT | | |
| 84 | Oligo 86 | AAGAGGTGGTGGAGGAGGTGGTGGAGGAGGTGGAGG | | |
| 85 | Oligo 87 | GGTGGGTGGTGGTGGG | | |

### Example 1.3

### The CpG Motifs in Oligo 4 are not Responsible for its Cytotoxic Effect

There are several classes of immunostimulatory oligonucleotides and these are broadly referred to as CpG oligonucleotide's. The immunostimulatory mechanism has been shown to involve the Toll-like receptor 9 (TLR9). It has evolved to recognize the presence of bacterial pathogens, exploiting the fact that unmethylated CpG motifs are much less frequent in mammalian genomes as compared to bacteria.

Many of the active oligonucleotides, in the context of this invention, contain several CpG motifs, although none appeared to have the optimum flanking sequences that have been documented by other researchers. For example, Oligo 4 contains 3 CpG motifs:

CGGGAGGAAGGCTAGCTACAACGAGAGGCGTTG(3′-3′T)

Therefore, the cytotoxicity of several variants of Oligo 4 were tested in the Cell-Titer Blue assay with HMEC-1 cells as described in other sections. It is demonstrated that these motifs do not account for its cytotoxic effect (3′-3′T denotes a 3′-inverted T modification).

a) Inhibition of Endosomal Maturation:

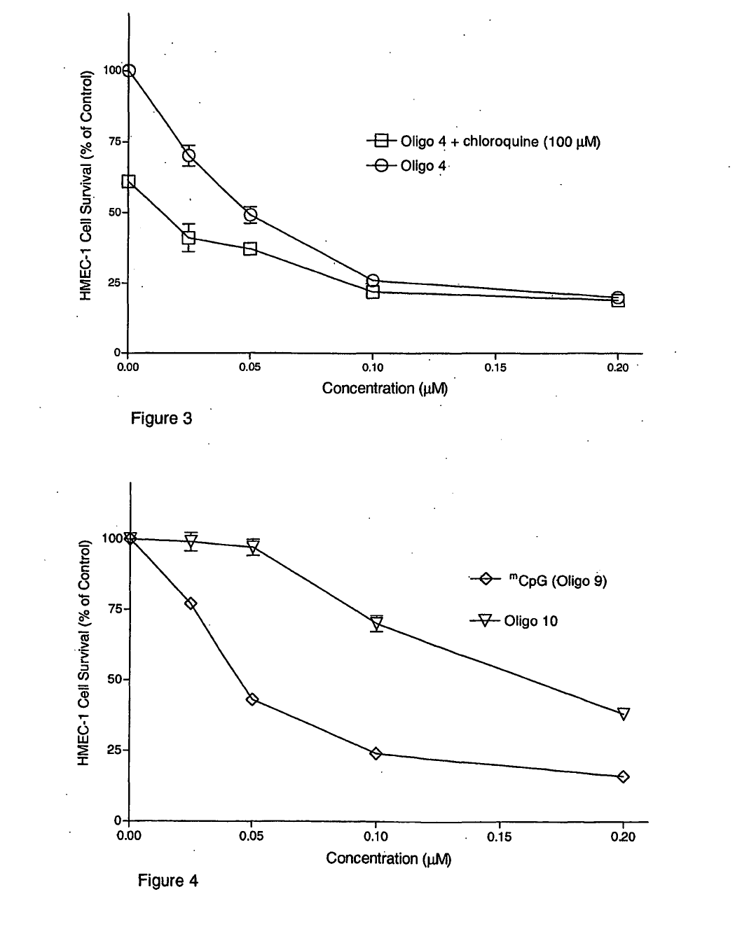

This was first ascertained by using chloroquine, an inhibitor of endosomal maturation. CpG ODN activation is dependent on internalization and endosomal maturation in macrophages and other immune cells. CpG DNA is recognized by toll-like receptor 9 (TLR-9), which is expressed mainly on the inner surface of endosomes (Hemmi et al, 2000). Oligo 4 retained substantial activity relative to the inactive control oligonucleotide (Oligo 7) in HMEC-1 cells preincubated with 25-100 μM chloroquine, although chloroquine alone was slightly toxic to the cells (FIG. 3).

The inability of chloroquine to diminish the activity of Oligo 4 (as shown in FIG. 3) suggests that TLR-9 is not involved in the cytotoxic cascade: Oligo 4 might, therefore, be binding to a different receptor thereby engaging a different pathway.

Moreover, HEK-293 cells do not express the TLR-9 receptor and these cells have been shown to be sensitive to CpG oligonucleotides only following transfection of this cell line with recombinant TLR-9. However, Oligo 4 was fully active in HEK-293 cells, indicating that the mechanism of toxicity of these novel oligonucleotides is independent of the CpG receptor TLR-9.

b) Methylation of CpG Cytosines:

Secondly, methylation of the CpG cytosines, which is known to prevent CpG immunostimulation (Goeckeritz, et al., 1999), produced an oligonucleotide (Oligo 9) with similar potency to Oligo 4. Oligo 10, in which two of the three CpG dinucleotides were inverted into GC dinucleotides

| | |
|---|---|
| Oligo 10: | [CGGGAGGAAGGCTAGCTACAAGCAGAGGGCTTG(3′- |
| | 3′T)], |

was less potent (FIG. 4), but nevertheless remained active.

c) Effect of CpG Oligonucleotides:

Thirdly, two CpG inhibitory ODNs were tested:

| | |
|---|---|
| Oligo 36: | TTAGGGTTAGGGTTAGGGTTAGGG(3′-3′T), |
| | and |
| Oilgo 37: | TCCTGGCGGGGAAGT(3′-3′T), |

Oligo 36 is a repetitive element in mammalian telomeres which blocks the colocalization of CpG DNA with TLR-9 within endosomal vesicles (Gursel, 2003). Oligo 37 is a CpG inhibitory sequence motif which blocks AP-1 transcriptional activation by CpG DNA (Lenert, 2003).

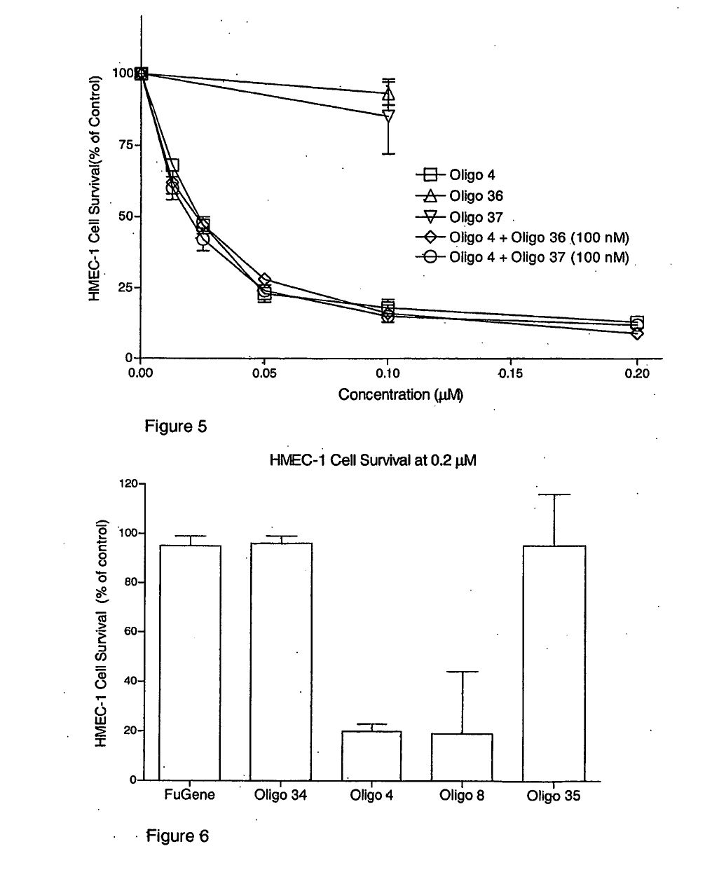

Both ODNs failed to inhibit the cytotoxic effect of Oligo 4 further demonstrating that CpG immune activation is not involved in the process (FIG. 5). Furthermore, neither was active on its own at the 100 nM concentration used.

### Example 1.4

### Essential Role of the G Triplet

This example demonstrates that the G triplet at the 5′end of the oligonucleotides is essential for cytotoxic activity.

a) Mutation Analysis:

Base mutation of the first guanosine of oligo 4 to thymine:

| | |
|---|---|
| Oligo 34: | CTGGAGGAAGGCTAGCTACAACGAGAGGCGTTG(3′- |
| | 3′T), |

yielded an oligonucleotide (Oligo 34) with no cytotoxic activity (FIG. 6).

Deleting two bases at the 5′ (which includes the first G of the triplet) and 3′ends:

| | |
|---|---|
| Oligo 35: | GGAGGAAGGCTAGCTACAACGAGAGGCGT(3′-3′T), |

also abolished this activity (FIG. 6; Oligo 35).

b) Formation of Tertiary Structures:

The involvement of the G triplet in the formation of tertiary structures such as G quadruplexes was tested by substituting either one of the three Gs in the triplet with 7-deaza guanine (7-dG), which inhibits the formation of Hoogsteen-type hydrogen bonds between the guanines (Beriimetskaya et al., 1997).

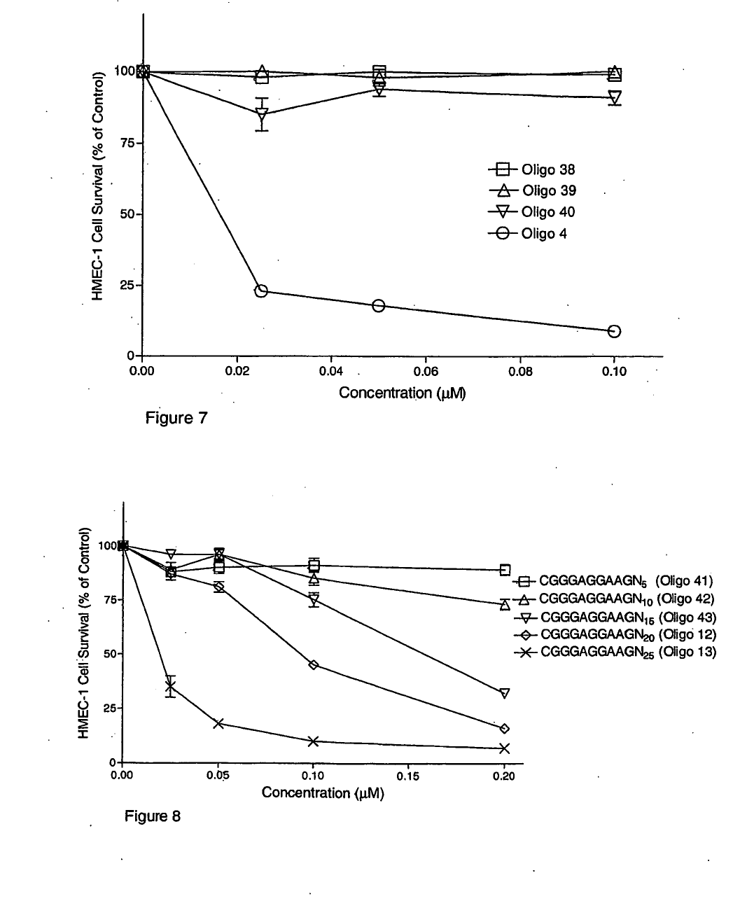

As shown in FIG. 7, ODNs presumably devoid of tertiary structures due to 7-deaza guanine substitutions (Oligos 38-40) had no suppressive activity as compared to Oligo 4 suggesting the possible role of higher order structures in the cytotoxic effect.

To further investigate the possible role of tertiary structures in the cytotoxic effect, circular dichroism studies, examining the rotational bias in the absorption of polarized light, were carried out. Solutions of G-quartet oligonucleotides have characteristic spectra when examined by circular dichroism (CD). Circular dichroism studies of Oligo 4 in solution were performed by Dr Max Keniry from the Australian National University. Solutions of Oligo 4 were prepared at a final concentration of 25 μmL (A₂₆₀=0.73) in 10 mM Tris pH 7.0 containing either 100 mM NaCl (Sample A) or 50 mM KCl (Sample B). Although previous experiments had indicated that the choice of monovalent cation during complexation did not impact on Oligo 4 cytotoxicity, the presence of each of K⁺ and Na⁺ was studied. Spectra were recorded at room temperature on a Jobin Yvon CD6 spectrometer using a 1 cm path length. Both samples demonstrated significant peaks at λ=218 and 275 nm, a trough at λ 245 nm and a cross-over point at λ 260 nm. This CD spectrum is typical of unstructured DNA, indicating the lack of formation of quadruplex structures under these conditions. Folded quadruplexes have a characteristic positive CD band at 295 nm and a negative band at 260 nm, whereas linear quadruplexes have a strong positive band at 260 nm and a negative band at 240 nm. Therefore, this indicates that the mechanism of cytotoxicity of Oligo 4 is different from that of other reported G-rich oligonucleotides, for which activity correlated closely with the ability to form G-quadruplex structures.

### Example 1.5

### The Cytotoxic Effect is Length-Dependent

Besides the requirement of a polyG motif at the 5′end, there also appears to be a length requirement for this cytotoxic effect. This was tested by adding random nucleotides to the first 10 bases of Oligo 4 (CGGGAGGMG). Results show that the cytotoxic effect was length-dependent; the potency of the ODN increased as the 3′end was extended with random bases (FIG. 8). ODNs≧30 bases in length demonstrated maximal activity (for example Oligos 12 & 13).

Similar results were obtained with the analogous variations to Oligo 1, with a reduced potency of the shorter tailed random oligonucleotide mixtures.

Finally, the same approach was taken using the sequence of GGGAGGAAAG as the 5′ sequence for random tailed oligonucleotide mixtures. Again, there was a length-dependence of the cytotoxic potential, with the 35 mer (Oligo 72) being the most potent.

### Example 1.6

### Sequence and Length Requirements for the 5′ Terminus

The sequence requirements for the 5′ terminus were further defined using 35 mer variants of Oligo 13, in which the 5′ sequence was increasingly replaced with random base mixes. Although Oligo 65, with only the first 4 bases of Oligo 4, still retained some activity, the requirement for a longer purine-rich stretch was seen with Oligo 63 and Oligo 13. In Oligo 65, although the defined G-rich region in the non-random part of the molecule is only 4 bases long, one skilled in the art would appreciate that a substantial proportion of this synthetic random pool would meet the required structural criteria. Indeed, by probability alone, one half of the random pool mix would satisfy the requirement for a 4 purine stretch containing one guanosine triplet.

Similar experiments with the first 10 bases of Oligo 1 (Oligo 66) provided further supporting data and, in particular, further stressed the need for 3 consecutive guanosines in this sequence. These results are shown in FIG. 28.

The fact that mixtures of random tailed oligonucleotides such as Oligo 13 or Oligo 66 are as active as Oligo 4 and Oligo 1, respectively, appears to indicate a lack of specific sequence requirements in the non 5′ sequence of these active oligonucleotides. However, Oligo 58 was inactive and Oligo 10 less active than Oligo 4, indicating that some tail sequences might have deleterious effects on the desired biological activity. Oligo 60, which has a G-free tail, was also substantially inactive. Oligonucleotides with tails consisting of ordered repeats of TAG (Oligo 70) and TAGGAT (Oligo 71), respectively also had reduced activity.

A series of oligonucleotides entirely composed of purines with repeated GGG sequences was tested. Again, there was a marked dependency on length with the 16 mer oligonucleotide (Oligo 77) having little activity. Although less active than Oligo 4 in HMEC-1, the 20 mer Oligo 78 had some activity, indicating that for some sequences, reduced length requirements might be obtainable. Nevertheless, Oligo 78 was clearly less potent than its longer congeners, particularly when tested with RSMC cells. The hexameric repeat oligonucleotide (Oligo 82) was more potent than Oligo 4 in HMEC-1 cells and had similar potency against the RSMC cells.

### Example 1.7

### Requirement for Single Strandedness

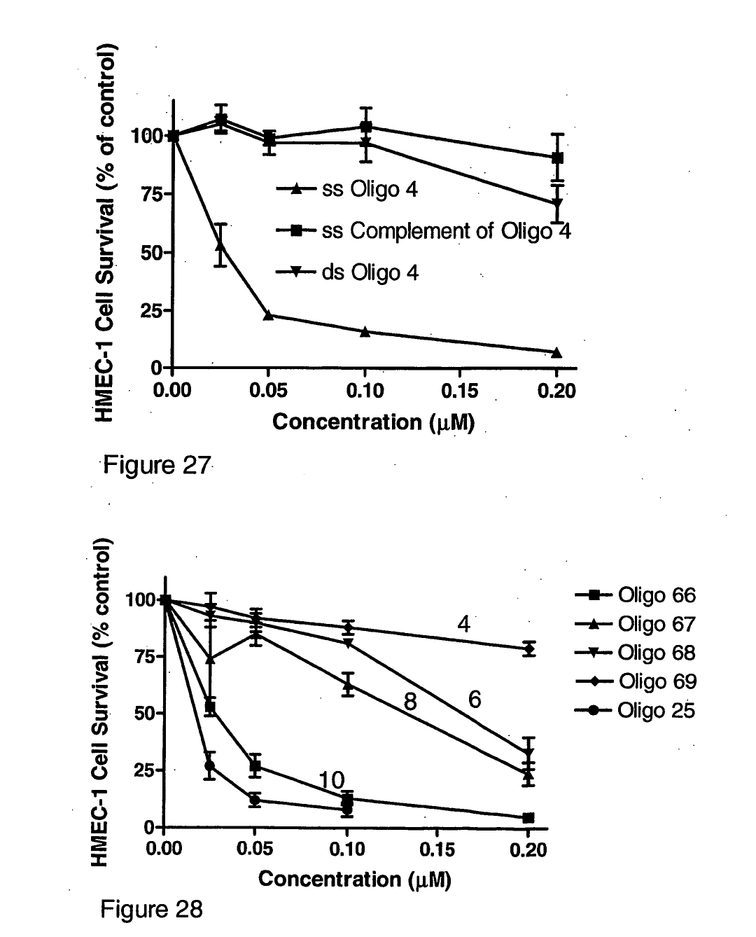

The requirement for single strandedness was investigated with Oligo 4. The complementary sequence to Oligo 4 (Oligo 61—inactive) was synthesised and annealed to Oligo 4 prior to complexation into HMEC-1 cells. As shown in FIG. 27, Oligo 61 and its duplex with Oligo 4 were both without significant activity as compared to Oligo 4 indicating that the mechanism of action of Oligo 4, and by inference other oligonucleotides of its class, is greatly suppressed when ODNs are present as fully double stranded duplexes.

Furthermore, oligonucleotides that are predicted to hybridize substantially in the 5′ region, i.e. form double-stranded regions within the 5′ region, could have diminished cytotoxic activity as exemplified by Oligo 47. Several available software programmes are able to predict DNA folding and these can be used to screen possible candidates for potential folding in this region, permitting elimination of such molecules.

### Example 1.8

### Cytotoxic Activity of the ODNs in Different Cell Lines

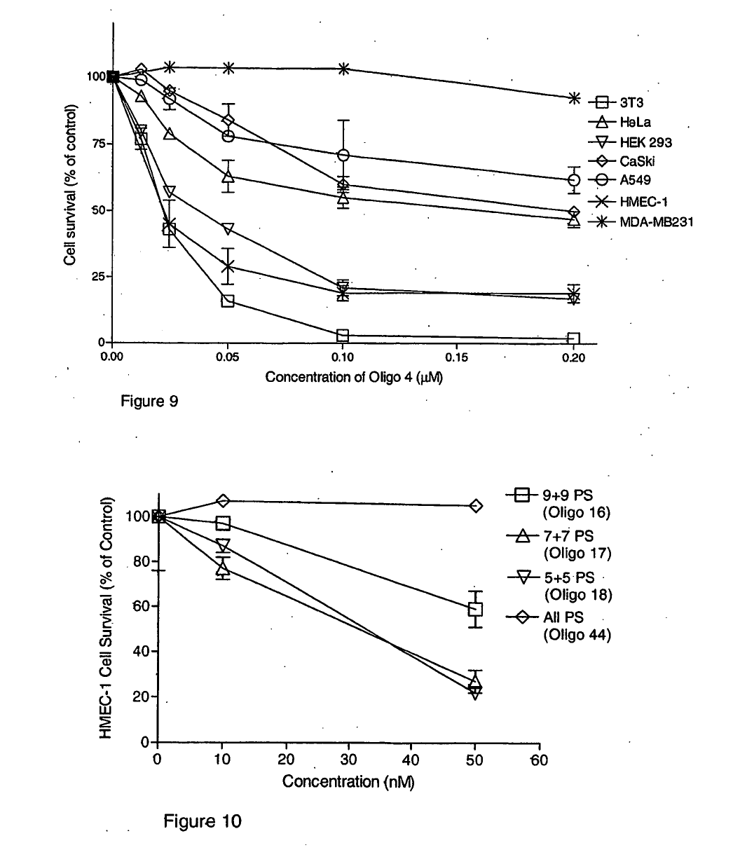

The activity of Oligo 4 was investigated in a variety of cell lines of different tissue- and species origin. The results are shown in FIG. 9.

Oligo 4 showed concentration-dependent cytotoxic activities in many of the cell lines tested which include mouse embryonic fibroblasts (3T3), transformed human embryonic kidney cells (HEK 293), human cervical cancer cell lines (HeLa and CaSki) and lung carcinoma (A549). A range in potency was observed in the human cancer cell lines tested.

### Example 1.9

### Chemical Modification of the Oligonucleotides

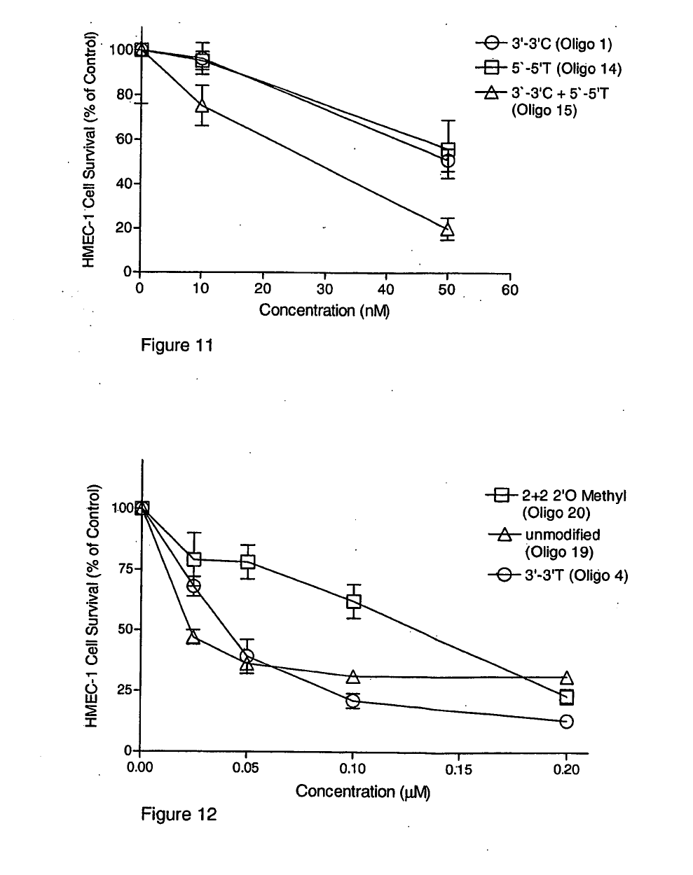

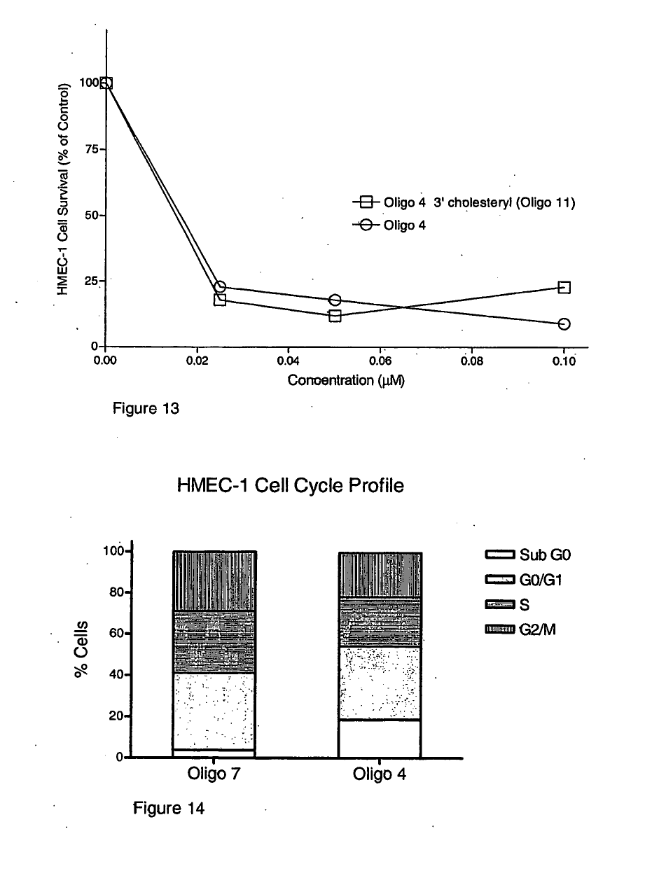

Chemical modifications designed to improve intracellular stability and uptake were envisaged to improve the cytotoxic activity of these ODNs. Results of the effect of different chemical modifications on Oligos 1 and 4 are presented in FIGS. 10-13.

Protecting the termini of the sequence from exonucleases by using partial phosphorothioate modifications (i.e. 5+5 PS denotes five phosphorothioate linkages at both 5′ and 3′ends; Oligo 18), and 5′ and 3′ inversions (Oligos 14 & 15) yielded active ODNs so that dose-dependent cytotoxic effects were maintained. A bulky 3′ modification such as cholesterol, designed for target cell-specific delivery, also preserved activity (Oligo 11). 2′O-methyl modifications were partially tolerated (Oligo 20) whereas total replacement of the phosphodiester backbone with phosphorothioate linkages (Oligo 44) greatly suppressed the activity.

### 2: Flow Cytometry Studies

### Example 2.1

### Induction of Cell Death

The HMEC-1 cell cytotoxicity work with Oligo 4 revealed morphological evidence of cell death at early times during the incubation (blebbing, nucleolar condensation) and by 48 hours, very few live cells remained in the wells treated at the high end of the concentration range. Extensive debris formation was observed in these wells. The survival decline in the presence of Oligo 4 could be due to induction of either cell necrosis or apoptosis. To decipher the specific mechanism, flow cytometry was employed in examining nuclear DNA content and measuring plasma membrane asymmetry and caspase activation. All studies were performed using HMEC-1 cells (1.2×10⁵ cells in a 6-well plate) transfected with either Oligo 4, or its “partially scrambled” counterpart which lacks the polyG motif, Oligo 7, GCGACGTGAGGCTAGCTACAACGAGTGGAGGAG(3′-3′T), complexed with Fugene6 at 100 nM final concentration. FuGENE6 reagent alone was used as the mock transfection control and FACS analyses were performed 24 h or 48 h post-transfection.

a) Cell-Cycle Analyses: ODNs Cause Accumulation of sub G₀ Cells:

For DNA cell cycle analyses, cells were harvested 24 h post transfection, permeabilised with 1% (v/v) Triton X-100/PBS and labeled with 10 μg/mI Propidium Iodide (PI). DNA content distribution was analysed and the percentage of cells in each distinct phase of the cell cycle is represented in FIG. 14. Cells that were transfected with Oligo 7 displayed a typical DNA profile of asynchronised cell growth, with very few cells in sub G₀ phase which represents the population of dead cells. In contrast, cells that were transfected with Oligo 4 displayed a significant increase from 3.7% to 18.6% in the sub Go population with coincident decrease in both S and G₂/M phase populations. These data suggest that Oligo 4 has caused HMEC-1 cell death, thus resulting in the accumulation of sub G₀ cells. Sub G₀ cells are considered to be cells in the terminal stages of programmed cell death.

b) Annexin V Analysis:

Forms of programmed cell death are often accompanied in the early stages by the translocation of the phospholipid phosphatidylserine (PS) from the inner to the outer leaflet of the plasma membrane. Once exposed extracellularly, PS can bind to Annexin V, a phospholipid binding protein with high affinity for PS. Later in programmed cell death, cells may become positive also for Propidium Iodide staining.

Cells were dual labelled with both FITC-conjugated antibody against Annexin V and Propidium Iodide (PI). PI intercalates double-stranded DNA of non-viable cells that have lost plasma membrane integrity as occurs in both necrosis and late stage apoptosis. Analyses of the dual labeled cells at both 24 and 48 h post transfection are represented as dot plots in FIG. 15. In this Figure, six analyses are shown in groups of two relating to analyses performed at 24 h (top row) and 48 h (bottom row) post-transfection with (from left to right) mock, Oligo 7 and Oligo 4, respectively. Treated cells were harvested, washed and analysed by flow cytometry after dual staining with labeled Annexin V and propidium iodide. The X and Y-axes represent the intensity of staining of individual cells to these latter two labels, respectively. At both time points, the mock and Oligo 7 transfected cells showed low percentages (2-6%) of cells undergoing programmed cell death (represented by lower and upper right hand quadrants—positive for Annexin V only, early programmed cell death, or for both Annexin V and PI, late programmed cell death, respectively). Furthermore, there was little indication of necrotic cell death (negligible number of cells in the upper left quadrant—positive for PI only). Cells that were transfected with Oligo 4 induced programmed cell death either at an early phase (lower right quadrant) or at late stages (upper right quadrant). After 24 h, 21% of cells underwent programmed cell death of which 10% were in early phase and 11% in late phase. This increased to 13% and 14% respectively at 48 h, representing a total of 27%. These results demonstrate that the cytotoxic effect of Oligo 4 had features of programmed cell death although a percentage of cells appeared necrotic.

c) Activation of Caspases:

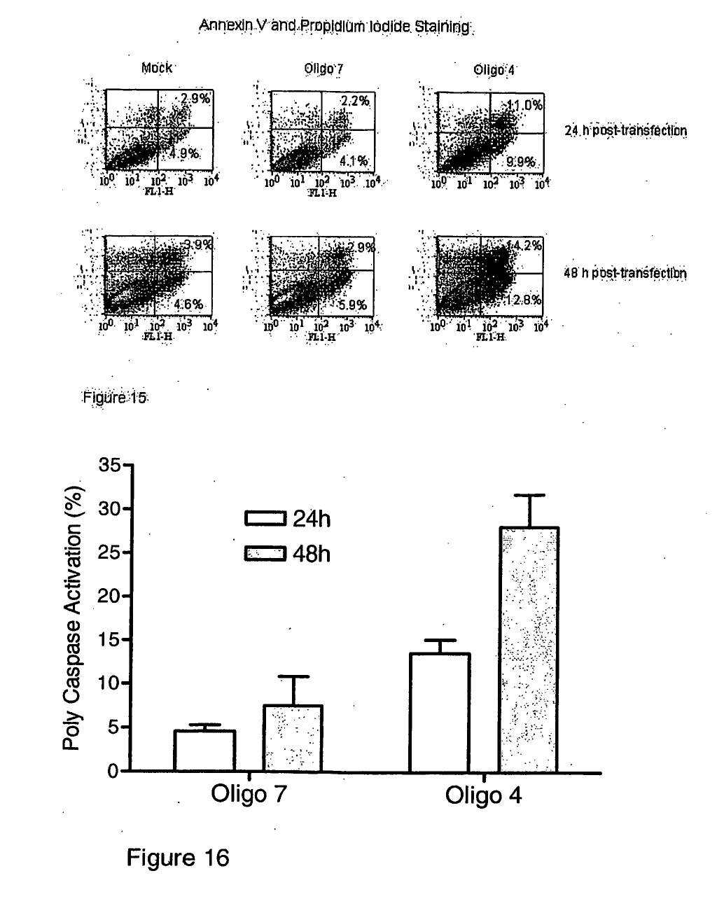

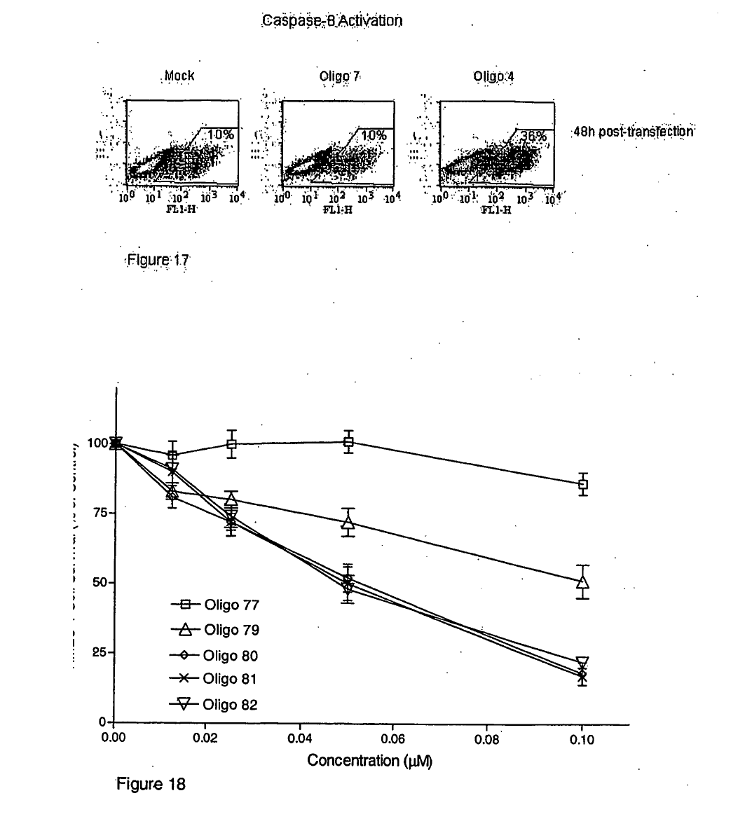

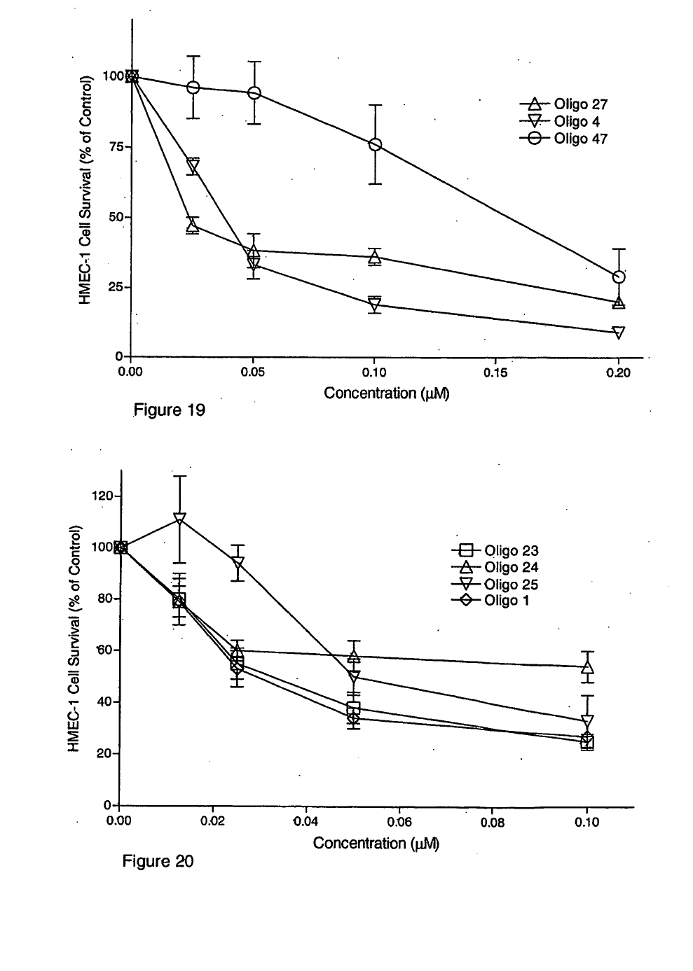

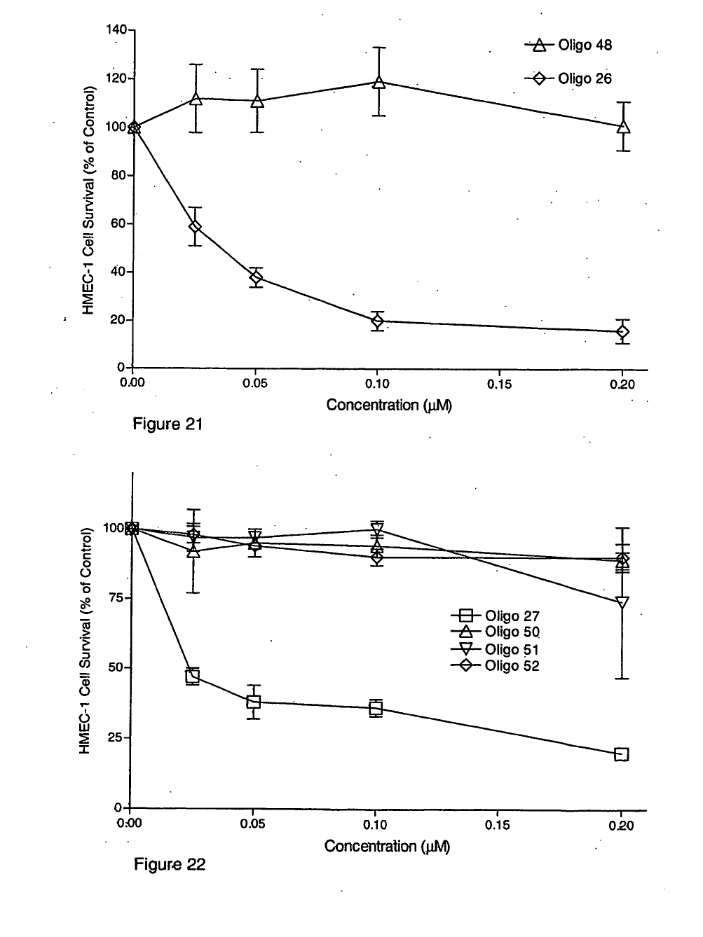

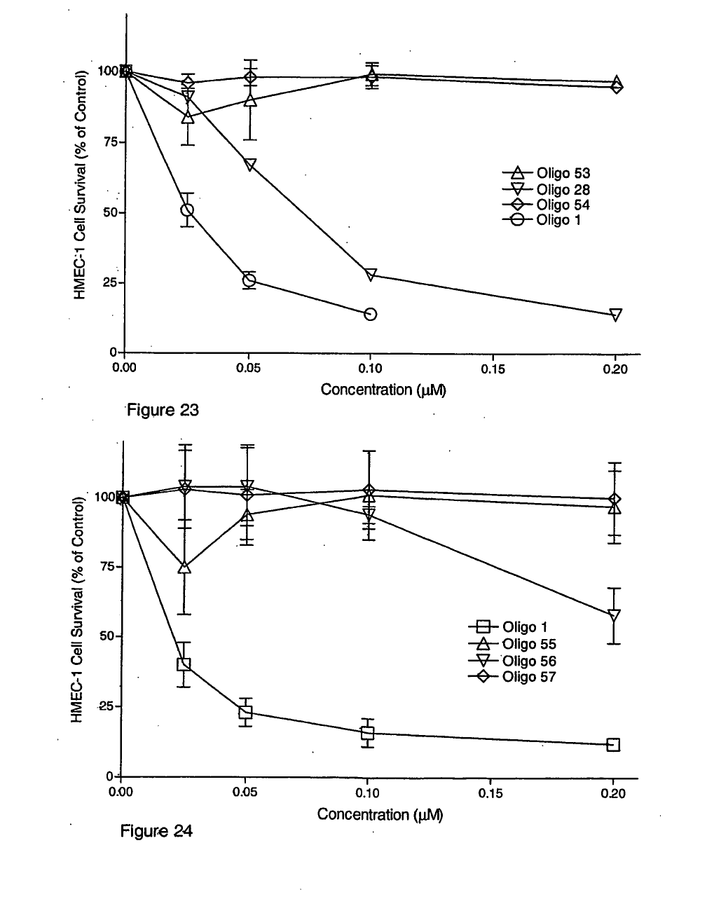

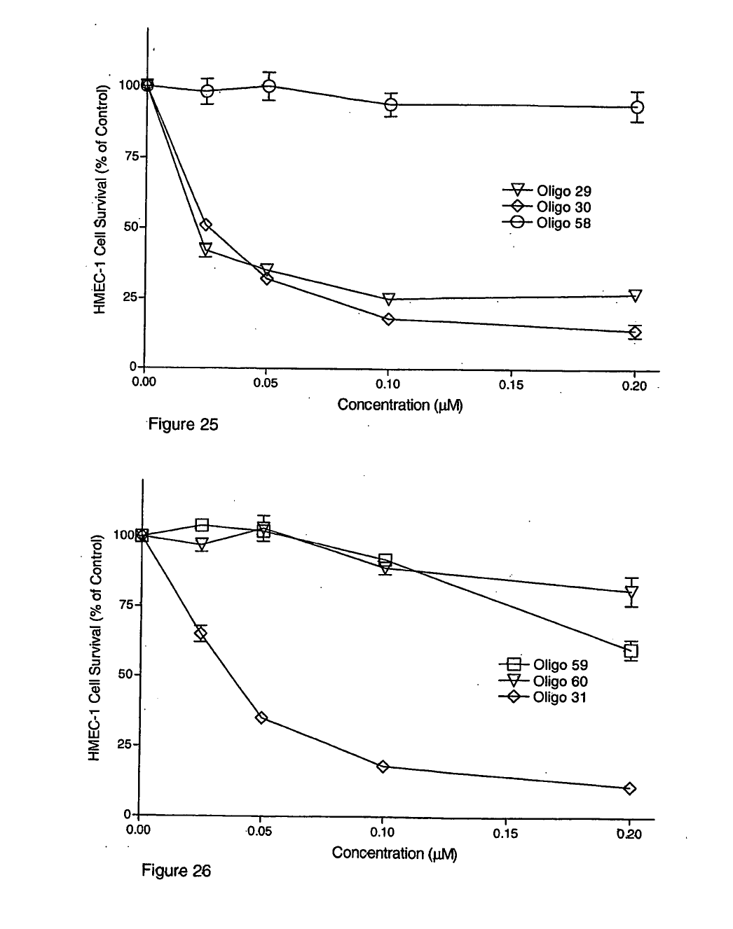

Apoptosis, a form of programmed cell death, usually involves activation of caspases in the death signaling pathways. In a concurrent set of experiments, transfected cells were stained with CaspACE FITC-VAD-FMK (Promega, Wis.), a polycaspase substrate which irreversibly binds to the intracellular active site of caspases. As shown in FIG. 16, Oligo 7—treated cells showed low ˜8% caspase activation as compared with the 14% and 28% caspase activation of cells transfected with Oligo 4 (24 h and 48 h post-transfection, respectively).

To investigate the potential involvement of initiator caspases -8 and -10 (which are immediately downstream of the death receptors Fas, TRAIL and TNFR1) through their recruitment via the death effector domain of the receptor signaling complex, FAM-conjugated caspase substrates (Immunochemistry Technologies, Minn.), LETD-FMK (caspase-8) and AEVD-FMK (caspase-10) were used and transfected cells were analysed. Results are shown as dot plots in FIG. 17. In FIG. 17, the results of flow cytometry are shown for three representative analyses with (from left to right) cells treated for 48 h with Mock, Oligo 7 and Oligo 4. The X-axis represents the intensity of staining to fluorescent LETD-FMK as a marker of caspase-8 activation. The Y-axis is the conventional side-scatter channel. Those cells that are located within the polygon R1 are considered positive for activated caspase-8.

Mock and Oligo 7 transfected cells showed a basal 10% caspase-8 activation. Cells transfected with the polyG rich Oligo 4, however, showed a significant increase in caspase-8 activation reaching approximately 36%. No significant activation of caspase-10 was observed following mock, Oligo 4, or Oligo 7′transfection (data not shown) ruling out the involvement of caspase-10 in the cascade. The observed induction of the cell death pathways by the polyG rich Oligo 4 may therefore be accompanied by caspase activation, more specifically by initiator caspase-8.

### Example 2.2

### Mitochondrial Depolarisation

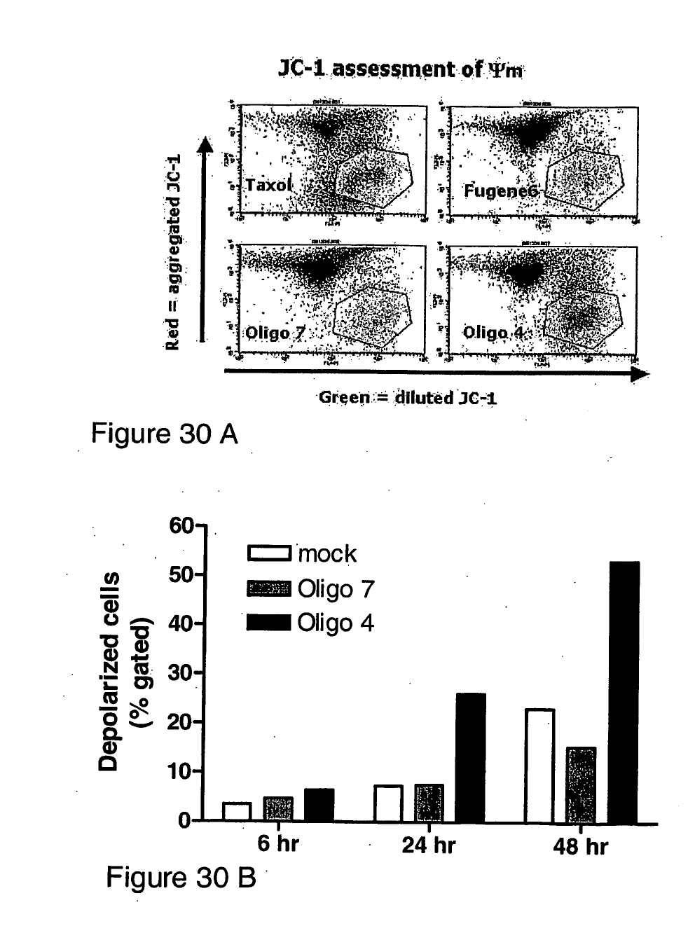

The role of mitochondrial depolarization in the death of treated cells was investigated by FACS using the mitochondrial dye JC-1. Representative analyses are shown in FIG. 30A. These analyses demonstrate the intensity of “red” (Y-axis) and “green” (X-axis) light emitted by JC-1 in cells treated for 48 h with (clockwise from upper left) taxol, mock (ie Fugene 6), Oligo 4 and Oligo 7. Cells emitting “green” light are considered to have disaggregated JC-1 as a result of mitochondrial depolarization and are shown gated in the, irregular hexagonal polygon. Taxol was included as positive control for mitochondrial-depolarization. Some evidence of mitochondrial depolarization could be observed in HMEC-1 cells as early as 6 hours post transfection with Oligo 4, but this increased with time with greater than 50% of cells showing mitochondrial depolarization 48 hours post-transfection (FIG. 30B). In comparison, the depolarization with oligo 7 was comparatively less than with oligo 4.

Both Oligo 82 and Oligo 79 induced mitochondrial depolarization to a similar extent as Oligo 1 and Oligo 4, suggesting that these oligonucleotides all share a common pathway of inducing cell death.

Using a NFkB-luciferase reporter plasmid, it was found that Oligo 4 reduced NFkB signalling in HMEC-1 cells when transfected 18 hours prior to evaluation of luciferase activity. This effect was also observed when HMEC-1 cells were stimulated with IL-1 beta and TNFalpha 5 hours prior to the luciferase reading.

Also, ICAM-1 protein expression on the surface of HMEC-1 cells, when induced by IL-1beta for 5 hours, was found to be inhibited by Oligo 4 when transfected into the cells 18 hours prior.

Because NFkB and ICAM-1 are important mediators of inflammatory response and leucocyte migration, this indicates that inter alia, these active oligonucleotides may be of use in clinical disorders in which inflammation is important. Also, Oligo 4 Induced the cell surface expression of FasL relative to the inactive oligonucleotide control (Oligo 7). Because FasL is deregulated in ocular angiogenesis, this indicates potential utility of the oligonucleotides of the invention in the treatment of AMD and diabetic retinopathy.

3. The ODNs of the Invention have Insignificant Cytotoxic Activity on Quiescent Cells

This example demonstrates that cells were most sensitive to the effects of Oligo 1 and Oligo 4 when grown under conditions of exponential growth.

HMEC-1 cells are known to exhibit some degree of contact inhibition and when seeded at 50,000/ well rather than the conventional 4,000/well, they formed a dense multilayer. Oligo 4 had no significant activity under these conditions.

This was shown not to be due to reduced transfection of the oligonucleotide with an Oregon green labelled Oligo 4. By FACS, transfection efficiency was ˜60% at 24 hours with both seeding densities.

A similar inhibition of activity of Oligo 4 due to contact inhibition was observed with two other cell lines, namely murine 3T3 cells and human ARPE-19 cells (human retinal pigmented epithelium).

Specifically, ARPE-19 cells were seeded at “low” and “high” densities of 4,000 and 50,000 cells per well, respectively. In the “high” seeding density wells, the ARPE cells rapidly reached a contact-inhibited quiescent state as determined by the formation of an organised monolayer.

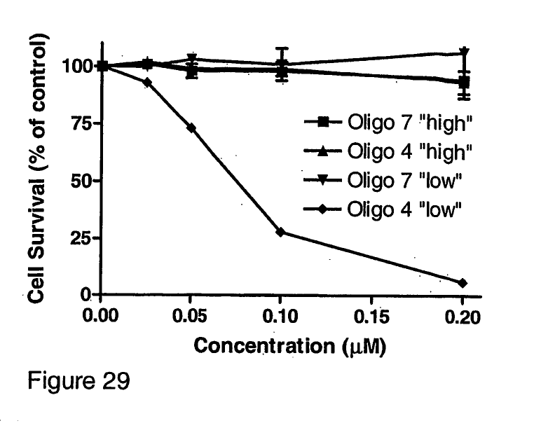

Oligonucleotides bearing the motif disclosed in the invention (eg Oligo 4) had potent activity against the rapidly dividing “active” cells when they were incubated for 48 hours with varying concentrations of oligonucleotide. In contrast, the effect was abolished (at concentrations <0.2 microM) for cells that were quiescent (FIG. 29).

Inactive oligonucleotides (eg Oligo 7) did not have appreciable activity in either set of conditions. Similar results were obtained with 3T3 (murine fibroblasts) which also attain contact-inhibited quiescence. This property indicates that cells are more susceptible to the oligonucleotides of the invention under conditions of active proliferation and/or migration. The oligonucleotides may have utility in disorders characterized by abnormal RPE, endothelium and fibroblast proliferation such as angiogenesis, proliferative retinal vitreopathy and scarring, granuloma etc.

### 4. Binding Of Oligonucleotides To Elongation Factor 1 Alpha 1

The binding protein sensor for Oligo 4 was identified as follows.

A 3′biotinylated analog of Oligo 4 was synthesized:

| | |
|---|---|
| Oligo 83: | CGGGAGGAAGGCTAGCTACAACGAGAGGCGTTG- |
| | B (B = biotin), |

and tested for cytotoxic activity as described previously. This oligonucleotide had equal activity against HMEC-1 cells.

In a separate experiment, total protein lysates of HMEC-1 cells were prepared in 2 mL MPER extraction buffer (Pierce, Rockford, Ill.). Streptavidin affinity beads were washed twice and resuspended in 2× affinity buffer (10 mM Tris-HCl, 1 mM EDTA, 2M NaCl (pH 7.4) and incubated for 10 minutes with an equal volume of 2 microM Oligo 83. The beads were then washed three times with binding buffer (20 mM HEPES, 100 mM KCl, 0.2 mM EDTA, 0.01% NP-40 and 10% glycerol, pH 7.5) and incubated with the protein lysate for 10 minutes at room temperature. The beads were washed 20 times with binding buffer and non-specific binding proteins eluted with two washes of a 1 microM solution of the non-cytotoxic oligonucleotide (Oligo 7) in binding buffer with alternating buffer washes.

Oligo 4 binding proteins were then eluted with two aliquots of Oligo 4 (1 microM) alternating with binding buffer washes. Aliquots of the Oligo 4 elutions as well as the Oligo 7 washes were concentrated (10000 mwt Centricon, 13800 g, 15 oC, 70 min) and electrophoresed under denaturing conditions on a gradient (4-12%) polyacrylamide gel. The gel was silver stained. Elutions with the inactive Oligo 7 yielded a large number of bands including a predominant band at ˜39 kDa. In contrast, the elution with Oligo 4 produced an intense-staining band at ˜51 kDa as well as 4-6 minor bands. The major band was identified to be eukaryotic elongation factor 1 alpha 1 by mass spectrometric analysis of a trypsin digested sample.

A further correlation for the binding of the active oligonucleotides, as defined in this invention, relative to those with little or no cytotoxic activity was sought. Briefly, beads prepared with Oligo 83 and processed as above with cell proteins were washed as above with Oligo 7. The beads fraction was then split 6 ways. These fractions were then incubated with 0.5 mL solutions of Oligo 4 (active), Oligo 1 (active), Oligo 34 (inactive variant of Oligo 4), Oligo 82 (purine-only active), as well as an additional oligonucleotide:

| | |
|---|---|
| Oligo 85: | TGTTTGTTTGTTTGTTTGTTTGTTTGT |

Oligo 85 is a 27-mer oligonucleotide capable of binding to a nuclear, basic and cancer-specific isoform of eEF1alpha1 (Dapas et al, Eur. J. Biochem, 270: 3251-3262).

Beads eluted with Oligos 4, 1 and 82 all produced a strong 51 kDa band, whereas Oligos 34 and 85 did not. This supports a possible correlation between cytotoxic activity and ability to bind at the Oligo 4 binding site of eEF1alpha1. Although Oligo 85 has been reported to bind to a cancer-specific isoform of this protein, it did not displace Oligo 4 from the affinity beads. This indicates that the binding of Oligo 85 to eEF1alpha1 is either isoform specific or is at a site distinct from that for Oligo 4. This is further supported by the fact that Oligo 85 was found to be inactive in HMEC-1 cells.

### Example 5

### Identification of Cytotoxic Oligonuleotides According to the Invention

The following tests (a) to (e) are used in determining whether oligonucleotides fitting the design rules of the invention have the capacity to induce cell death according to the invention

a) Cytotoxicitv Assay:
Active oligonucleotides are identified by testing these oligonucleotides for cytotoxic activity against SV-40 transformed human dermal microvascular endothelial (HMEC-1) and rat vascular smooth muscle (RSMC) cell lines, which can be obtained from the ATCC.
HMEC-1 cells are maintained in MCDB131 medium containing 10% fetal bovine serum (FBS), 2 mM L-glutamine, 10 ng/mL epidermal growth factor, 1 μg/mL hydrocortisone and 5 U/mL penicillin-streptomycin.
The RSMC cells are grown in DMEM F12 containing 10% FBS, 2 mM L-glutamine and 5U/mL penicillin-streptomycin. It should be appreciated by one skilled in the art that minor modifications to these culture conditions could be envisaged and that these may or may not modify the activity of the said oligonucleotides.
The oligonucleotides should be of high quality and preferably purified by reverse-phase chromatography to avoid accidental contamination with synthetic impurities which might cause non-specific toxicity. Likewise, the oligonucleotides to be tested must be devoid of microbiological contamination. Pure oligonucleotides can be reconstituted in pure water at a concentration suitable for subsequent dilution and use such as 50 microM.
Cytotoxicity assays are performed as follows: Cells are seeded at 4000 cells per well in 96-well black MicroClear plates (Greiner). After 24 hours, HMEC-1 cells in growth medium containing 5% FBS or RSMCs in growth medium containing 10% FBS are transfected with a range of concentrations of the oligonucleotides (0-400 nM) in triplicate using FuGENE6 (Roche). A FuGENE6: DNA ratio of 3:1 (μL FuGENE6/μg DNA) is particularly useful in testing oligonucleotides in these two cell lines because it is devoid of toxicity used for all transfections. The oligonucleotide complexation with FuGENE6 reagent can be performed in Optimem, complete medium or similar, without compromising complexation efficiency. Complexation can be performed with the oligonucleotide at an initial concentration of 2 μM and the DNA complex can then be serially diluted to conveniently generate a range of stock concentrations required to generate the final medium concentrations of oligonucleotide upon addition to cells in culture medium. Medium does not need to be changed prior to assessment of cell viability.
Cell survival is assessed 48 hours post-transfection using a fluorometric cell viability assay (CellTiter™-Blue Cell Viability Assay; Promega). Media in wells are replaced with 100 μL OptiMEM to which 20 μL of the assay mix is added. After 2 hours at 37° C., fluorescence is measured at 544_{Ex}/590_{Em} using FLUOstar OPTIMA (BMG Labtechnologies). One skilled in the art will appreciate that there are many other methods for assessing cell survival and cytotoxicity including direct counting with Trypan Blue exclusion and other colorimetric and fluorometric assays comparable with the CellTiter Blue Assay. The latter, however, is sensitive and produces reproducible results when assaying active oligonucleotides as described in this invention.
Results are normalized according to untreated cells (100%). Active oligonucleotides demonstrate significant, reproducible and concentration-dependent cytotoxicity over the range 0-100 nM in non-confluent HMEC-1 and SV40 RSMC cells when transfected with Fugene 6. Significant cytotoxicity means that at concentrations of 100 nM there is at least 20% reduction in cell survival compared to mock-transfected controls; preferably at least 25%.
One skilled in the art will appreciate that the results may vary from occasion to occasion and that tests should be repeated, for example at least twice, and preferably at least three times, to establish the significance of any result.
Furthermore, the active oligonucleotides described in the invention have greatly reduced efficacy against cells that have been grown to high levels of confluence. It will be appreciated that seeding densities of cells may need to be adjusted as required to ensure that confluence of cells at time of incubation with the oligonucleotides is not in excess of 40-50%. Variability in cell proliferation may be encountered due to differences in passage number, batch of serum used in the medium and additional incubation factors (glutamine, type of culture ware etc).

b) Assessment of Microscopic Signs of Cell Death:
According to the invention, cytotoxic activity against HMEC-1 and RSMC cells is accompanied by obvious microscopic signs of cell death with shrinking and detachment and formation of cell debris after 24 and 48 hours incubation at high concentrations (200 nM). Light microscopy is a suitable technique for detecting these changes.

c) Assessment of Depolarization of Mitochondria
In HMEC-1 cells, death is accompanied by depolarization of the mitochondria. This can be conveniently assessed by seeding 1.2×10⁵ cells/well in a 6-well cell culture plate. Cells are transfected 24 hours later with the oligonucleotide after complexation in Fugene6 as described above at a concentration sufficient to induce significant cell death (50-200 nM).
Cells are trypsinized and harvested 12-48 hours later, making sure that detached cells are also collected. The cells are incubated with JC-1 or similar. One skilled in the art would appreciate that JC-1 can be substituted for another appropriate marker of mitochondrial potential. Cells are then arialysed in a Fluorescence Activated Cell Sorter (FACS) to determine the frequency of cells demonstrating green-shifted fluorescence. An active oligonucleotide as described in this invention will, at concentrations capable of inducing. cytotoxicity, cause significant depolarization of mitochondria relative to untreated or mock-transfected HMEC-1 cells.
As described above, one skilled in the art will rapidly identify oligonucleotides that do not fit the design rules and that demonstrate a reproducible lack of cytotoxic activity when tested in HMEC-1 and RSM cells under the conditions described above. It is recommended that one or more of these inactive oligonucleotides be included in the experiments examining mitochondrial depolarization to ensure that the effects seen are robust and specific to active oligonucleotides.

d) Activation of Casoases:
Other signs of cytotoxicity can be detected as part of the testing of the oligonucleotides described in the invention. Amongst these, activation of caspases can be determined.
HMEC-1 cells are seeded into Swell plates (1.2×10⁵ cells/well) and transfected with 50-200 nM of the relevant oligonucleotide or the equivalent amount of Fugene6 alone 24 hours later. Cells are harvested by mild trypsin-EDTA digestion and stained with 10 μM CaspACE™ FITC-VAD-fmk (Promega). The cells are washed again after 2 hours and re analyzed for fluorescence by FACS. Oligonucleotides described in this invention have the ability of inducing significant caspase activation above and beyond the baseline caspase activity in HMEC-1 cells.

e) Staining with Annexin V:
The active oligonucleotides of the invention cause the exposure of the inner membrane phosphatidyl serines in HMEC-1 cells. This property is easily studied by staining cells harvested in the above-mentioned manner with 0.5 μg/ml FITC labeled recombinant annexin V. The cells can be simultaneously stained with 0.6 μg/ml propidium iodide (Oncogene Research Products) so that the various populations of cells with characteristic staining can be estimated.
Positivity of staining with Annexin V is considered characteristic of programmed forms of death such as apoptosis and autophagy. Cells that are positive only for propidium iodide staining are understood to be undergoing necrotic cell death.
HMEC-1 cells incubated with oligonucleotides that are disclosed in this invention have significantly increased staining with annexin V and propidium iodide.

Although the above mentioned tests (a) to (e) are described using HMEC-1 cells, several other readily cultured cell lines can be used to demonstrate the activity of the oligonucleotides disclosed in this invention. Notably, 3T3 fibroblasts, HEK293, HeLa, PC3 are amongst those in which the activity of the said oligonucleotides is exhibited. In contrast, the oligonucleotides described do not demonstrate cytotoxicity over the 0-200 nM concentration range when transfected into either human colon carcinoma cells HCT-116 and human breast cancer cells MbA-MB-231 using Fugene6 with the previously described conditions.

### Example 6

### Evaluation of In vivo Cytotoxicity of G-rich Oligonucleotides of the Invention

Cytotoxic ODNs can be further evaluated in vivo for inhibition of disease-related angiogenesis according to a number of validated preclinical models. For example, in the area of ocular angiogenesis, a widely-recognized model is the laser-induced Choroidal Neovascularization (CNV) model in rats.

Accordingly, a number of rcs/rdy⁺ pigmented rats are obtained. Rats are housed in cages at a constant temperature of 22° C, with a 12:1.2 hour light/dark cycle (light on at 0800 hours) and food and water are made available ad libitum. Rats are anaesthetised by intramuscular injection of xylazine (6 mg/kg, Bayer AG, Germany) and ketamine (50 mg/kg, Lambert Company, USA) injection. The. pupils are dilated with 2.5% phenylephrine and 1% Mydriacyl at least 10 minutes before photography and or laser photocoagulation.

Choroidal neovascularisation (CNV) is induced by krypton laser photocoagulation. This is performed using laser irradiation to either the left or alternatively, the right eye of each animal from all treatment groups through a Zeiss slit lamp. A total of 6-11 laser burns are applied to each eye surrounding the optic nerve at the posterior pole at a setting of 100 μm diameter, 0.1 seconds duration and 150 mW intensity.

At a suitable time following laser injury, the oligonucleotides are injected into the affected eyes. The suitable time can be the day following laser induction, or for an assessment against established CNV, the injections can be performed several days or weeks following injury. Intravitreal injections of the oligonucleotides are performed by inserting a 30- or 32-gauge needle into the vitreous at a site 1 mm posterior to the limbus of the eye. Insertion and infusion can be performed and directly viewed through an operating microscope. Care is taken not to injure the lens or the retina. Ideally, the test compounds are placed in the superior and peripheral vitreous cavity. An injection volume of 1 microlitre is appropriate.

Periodically after treatment, the neoangiogenesis is evaluated by either imaging and/or direct sampling (eg histology, immunohistochemistry). In all cases, the assessment of CNV is best performed by a skilled operator blinded to the actual treatment to ensure a lack of bias in the recording of the Information.

An example of a direct imaging method is Colour Fundus Photography (CFP). Again, under anaesthesia as described above, the pupils are dilated with 2.5% phenylephrine and 1% Mydriacyl at least 10 minutes before photography. The rat fundus is then photographed with a small animal fundus camera using the appropriate film.

Alternatively, or preferably in addition to FCP, fluorescein angiography is used to image the vessels and areas of vascular leakage in the retina. This is performed on all of the rats following the intraperitoneal injection of 0.3 to 0.4 ml 10% sodium fluorescein. The retinal vasculature is then photographed using the same camera as used for FCP but with a barrier filter for fluorescein angiography added. Single photographs can be taken at 0.5-1 minute intervals using monochrome Kodak 400 ASA professional film immediately after the administration of sodium fluorescein. The extent of fluorescein leakage is scored by a trained operator or alternatively, by other methods known in the art for measuring leakage. The mean severity scores from each of the time points are compared by ANOVA with a post hoc Fishers LSD analysis and differences considered significant at p<0.05. In addition, the frequency of each lesion score is counted, tabulated and represented graphically.

Rats treated with active oligonucleotides according to the present invention are expected to show a significantly lower severity score than control animals. Alternatively, or in addition, rats can be euthanased at selected time points following treatment (for example 7, 14 and 28 days post injection) with an overdose of sodium pentabarbital. For paraffin sectioning, eyes are enucleated and fixed for 4 hours in 10% neutral buffered saline or 4% paraformaldehyde. After routine processing through graded alcohol, the eyes are embedded in paraffin and sectioned at 5 μm, mounted on sialinated slides and stained with haematoxylin and eosin (H&E) for histopathological examination. A reduction in the number and severity of lesions is expected to be seen with samples treated by active oligonucleotides of the invention.

While the invention has been described in terms of various preferred embodiments, the skilled artisan will appreciate that various modifications, substitutions, omissions and changes may be made without departing from the scope thereof. Accordingly, it is intended that the scope of the present invention be limited by the scope of the following claims, including equivalents thereof.

### REFERENCES

Anfossi G, Gewirtz A M and Calabretta B. 1989: An oligomer complementary to c-myb-encoded mRNA inhibits proliferation of human myeloid leukemia cell lines. Proc. Natl. Acad. Sci. USA 86: 3379-83.

Benimetskaya L, Berton M, Kolbanovsky A, Benimetsky S. and Stein C A. 1997. Formation of a G-tetrad and higher order structures correlates with biological activity of the RelA (NF-kappaB p 64) “antisense” oligonucleotide. Nucleic Acids Res. 25: 2648-2656.

Burgstaller P, Jenne A and Blind M. 2002. Aptamers and aptazymes: accelerating small molecule drug discovery. Curr. Opin. Drug Discov. Devel. 5: 690-700.

Cotter F E, Johnson P, Hall P, Pocock C, Al Mahdi N, Cowell J K and Morgan G. 1994. Antisense oligonucleotides suppress B-cell lymphoma growth in a SCID-hu mouse model. Oncogene 9: 3049-55.

Dapas et al., 2003, Eur. J. Biochem., 270: 3251-3262.

Ejiri S. I., 2002, Biosci. Biotechnol. Biochem. 66(1), 1-21.

Goeckeritz B E, Flora M, Witherspoon K, Vos Q, Lees A, Dennis G J, Pisetsky D S, Kloinman D M, Snapper C M and Mond J J. 1999. Multivalent cross-linking of membrane Ig sensitizes murine B cells to a broader spectrum of CpG-contgaining oligodeoxynucleotide motifs, including their methylated counterparts, for stimulation of proliferation and Ig secretion. Int. Immunol. 11:1693.

Gursel I, Gursel M, Yamada H, lshii K J, Takeshita F and Klinman D M. 2003. Repetitive elements in mammalian telomeres suppress bacterial DNA-induced immune activation. J. Immunol. 171:1393-1400.

Guvakova M A, Yakubov L A, Vlodavsky I, Tonkinson J L and Stein C A. 1995. Phosphorothioate oligodeoxynucleotides bind to basic fibroblast growth factor, inhibit its binding to cell surface receptors, and remove it from low affinity binding sites on extracellular matrix. J. Biol. Chem. 270: 2620-27.

Hemmi H, Takeuchi O, Kawai T, Kaisho T, Sato S, Sanjo H, Matsumoto M, Hoshino K, Wagner H, and Akira S. 2000. A toll-like receptor recognizes bacterial DNA. Nature 408: 740-5.

Jueliger S and Bates P J. 2004. Expression of fluorescent-tagged nucleolin and co-localization with G-rich oligonucleotides in lung cancer cells. Proceedings of the AACR 45.

Khachigian L. et al. 2002, J. Biol. Chem., vol 277, n° 25, pp 22985-22991.

Lenert P, Yi A-K, Krieg A M, Stunz L L and Ashman R F. Inhibitory oligonucleotides block the induction of AP-1 transcription factor by stimulatory CpG oligonucleotides in B cells. 2003. Antisense Nucleic Acid Drug Dev 13: 143-150.

Sun L-Q. et al., 1999, J. Biol. Chem., vol 274, n° 24, pp 17236-17241.

Wu C C N, Lee J, Raz E, Corr M and Carson D A. 2004. Necessity of oligonucleotide aggregation for toll-like receptor 9 activation. J. Biol. Chem. 279: 33071-8

Wyatt J R, Davis P W and Freier S M. 1996. Kinetics of G-quartet-mediated tetramer formation. Biochemistiy 35: 8002-8.

Yaswen P, Stampfer M R, Ghosh K and Cohen J S. 1993. Effects of sequence of thioated oligonucleotides on cultured human mammary epithelial cells. Antisense Res. Dev. 3: 67-77.

Zhang G, et al., 2004, Journal of the National Cancer Institute, Vol. 96, n°9, pp 683-696

1. A method of treating or preventing a disorder involving aberrant cell proliferation or migration, comprising administering to a patient an oligonucleotide which induces, in non-quiescent eukaryotic cells, cell death,
wherein said oligonucleotide is to be administered to a subject in an amount such that it induces cell death having at least one characteristic of programmed cell death in said cells in said subject,
wherein said oligonucleotide has a length of 25 to 50 nucleotides and consists of
i) a 5′ G-rich region having 6 to 9 nucleotides, and
ii) a 3′ tail region,
wherein the 5′ G-rich region has the formula 1:

| | |
|---|---|
| 5′ [X¹-X²-(R¹-R²-R³-R⁴)-X³-X⁴-X⁵-X⁶-X⁷] 3′ | Formula 1 |

in which
(R¹-R²-R³-R⁴) represents a tract of four consecutive purine nucleotides, each R representing a purine nucleotide,
each of X¹, X², X³, X⁴, X⁵, X⁶ and X⁷ independently represents a nucleotide which may be present or absent, such that the total number of nucleotides in the G-rich region is from 6 to 9,
each of X¹, X² X³, X⁴, X⁵, X⁶ and X⁷ independently represents a purine or pyrimidine nucleotide, provided that:
at least 50% of the nucleotides in the G-rich region are guanosine nucleotides,
the portion of the G-rich region represented by X²-(R¹-R²-R³-R⁴) contains a triple guanosine motif (G-G-G),
the portion of the G-rich region represented by X³-X⁴-X⁵-X⁶-X⁷ does not contain a thymidine nucleotide downstream of a guanosine nucleotide,
the G-rich region is not composed exclusively of guanosine nucleotides,
the nucleotide defining the 3′ extremity of the G-rich region is a guanosine nucleotide,
the total number of pyrimidine nucleotides in the G-rich region does not exceed 2, and these pyrimidine nucleotides are not consecutive to each other,
and the 3′ tail region is any nucleotide sequence.

2. The method according to claim 1 wherein X¹ is present or absent, X2 is present and is a pyrimidine nucleotide (Py), and the 5′ G-rich region has the Formula 2:

| | |
|---|---|
| 5′ [X¹-Py-(R¹-R²-R³-R⁴)-X³-X⁴-X⁵-X⁶] 3′ | Formula 2 |

wherein R, X¹, X³ , X⁴, X⁵ and X⁶ have the previously defined meanings, the triple G motif (G-G-G) is present in the (R¹-R²-R³-R⁴) purine tract, and X³, X⁴, X⁵ and X⁶ may be present or absent such that the total number of nucleotides in the G-rich region is from 6 to 9.

3. The method according to claim 2 wherein Py in Formula 2 is a cytosine nucleotide.

4. The method according to claim 3 wherein the G-rich region comprises the sequence:
5′ GCGGGG 3′

5. The method according to claim 1 wherein X¹ and X² are absent, X³ represents a cytosine nucleotide and the 5′ G-rich region has the Formula 3:

| | |
|---|---|
| 5′ [(R¹-R²-R³-R⁴)-C-X⁴-X⁵-X⁶-X⁷] 3′ | Formula 3 |

wherein R, X⁴, X⁵, X⁶ and X⁷ have the previously defined meanings, the triple G motif (G-G-G) is present in the (R¹-R²-R³-R⁴) purine tract and X³, X⁴ X⁵, X⁶ and X⁷ may be present or absent such that the total number of nucleotides in the G-rich region is from 6 to 9.

6. The method according to claim 5 wherein the G-rich region comprises the sequence
5′ GGGGCAG 3′.

7. The method according to claim 1 wherein X¹ is present or absent, X² is present and is a purine nucleotide (R⁵), and the 5′ G-rich region has the Formula 4:

| | |
|---|---|
| 5′ [X¹-(R⁵-R¹-R²-R³-R⁴)-X³-X⁴-X⁵-X⁶] 3′ | Formula 4 |

wherein:
R, X¹, X³, X⁴, X⁵ and X⁶ have the previously defined meanings,
(R⁵-R¹-R²-R³-R⁴) represents a tract of five consecutive purine nucleotides,
the triple G motif (G-G-G) is present in the (R⁵-R¹-R²-R³-R⁴) purine tract, and
X³, X^{4,} X⁵ and X⁶ may be present or absent such that the total number of nucleotides in the G-rich region is from 6 to 9.

8. The method according to claim 7 wherein X¹ is present and the 5′ G-rich region has the Formula 5:

| | |
|---|---|
| 5′ [X¹-(R⁵-R¹-R²-R³-R⁴)-X³-X⁴-X⁵] 3′ | Formula 5 |

wherein:
R, (R⁵-R¹-R²-R³-R⁴), X¹, X³, X⁴ and X⁵ have the previously defined meanings, and
X³, X⁴ and X⁵ may be present or absent such that the total number of nucleotides in the G-rich region is from 6 to 9.

9. The method according to claim 7 wherein X¹ is absent, and the 5′ G-rich region has the Formula 6:

| | |
|---|---|
| 5′ [(R⁵-R¹-R²-R³-R⁴)-X³-X⁴-X⁵-X⁶] 3′ | Formula 6 |

wherein:
R, (R⁵-R¹-R²-R³-R⁴), X³, X⁴, X⁵ and X⁶ have the previously defined meanings, and
X³, X⁴, X⁵ and X⁶ may be present or absent such that the total number of nucleotides in the G-rich region is from 6 to 9.

10. The method according to claim 8 wherein the (R⁵-R¹-R²-R³-R⁴) tract is adenosine-containing, and the G-rich region has the formula 5.1:

| | |
|---|---|
| 5′ [X¹-(R⁵-R¹-R²-R³-R⁴)-X³-X⁴-X⁵] 3′ | Formula 5.1 |

wherein at least one of R⁵, R¹, R³ and R⁴ represents A,
X¹ represents a purine or pyrimidine nucleotide, and
X³, X⁴, and X⁵ have the previously defined meanings, and may be present or absent such that the total number of nucleotides in the G-rich region is from 6 to 9.

11. The method according to claim 10 wherein the 5′ G-rich region has 6 nucleotides and is chosen from the group consisting of:

| | |
|---|---|
| 5′ [X¹-(AGGGG)] 3′ | Formula 5.1.1 |
| 5′ [X¹-(GAGGG)] 3′ | Formula 5.1.2 |
| 5′ [X¹-(GGGAG)] 3′ | Formula 5.1.3 |

wherein A represents adenosine and G represents guanosine, and X¹ represents a purine or pyrimidine nucleotide.

12. The method according to claim 10 wherein the 5′ G-rich region has 7 to 9 nucleotides and is chosen from the group consisting of:

| | |
|---|---|
| 5′ [X¹-(GGGGA)-X³-X⁴-X⁵] 3′ | Formula 5.1.4 |
| 5′ [X¹-(AGGGA)-X³-X⁴-X⁵] 3′ | Formula 5.1.5 |

wherein A represents adenosine and G represents guanosine, X¹ represents a purine or pyrimidine nucleotide, X³, X⁴, and X⁵ have the previously defined meanings, and X⁴ and X⁵ may be present or absent such that the total number of nucleotides in the G-rich region is 7, 8 or 9.

13. The method according to claim 8 wherein the (R⁵-R¹-R²-R³-R⁴) tract is devoid of adenosine nucleotides and the G-rich region has the formula 5.2:

| | |
|---|---|
| 5′ [X¹-(G-G-G-G-G)] 3′ | Formula 5.2 |

wherein X¹ represents A, C or T.

14. The method according to claim 9 wherein the (R⁵-R¹-R²-R³-R⁴) tract is adenosine-containing, and the G-rich region has the formula 6.1:

| | |
|---|---|
| 5′ [(R⁵-R¹-R²-R³-R⁴)-X³-X⁴-X⁵-X⁶] 3′ | Formula 6.1 |

wherein at least one of R⁵, R¹, R³ and R⁴ represents A,
X³, X⁴, X⁵ and X⁶ have the previously defined meanings, and may be present or absent such that the total number of nucleotides in the G-rich region is from 6 to 9.

15. The method according to claim 14 wherein the 5′ G-rich region is chosen from the group consisting of:

| | |
|---|---|
| 5′ [(AGGGG)-X³-X⁴-X⁵-X⁶] 3′ | Formula 6.1.1 |
| 5′ [(GAGGG)-X³-X⁴-X⁵-X⁶] 3′ | Formula 6.1.2 |
| 5′ [(GGGAG)-X³-X⁴-X⁵-X⁶] 3′ | Formula 6.1.3 |
| 5′ [(GGGGA)-X³-X⁴-X⁵-X⁶] 3′ | Formula 6.1.4 |
| 5′ [(AGGGA)-X³-X⁴-X⁵-X⁶] 3′ | Formula 6.1.5 |

wherein A represents adenosine and G represents guanosine, and
X³, X⁴, X⁵ and X⁶ have the previously defined meanings,
and X⁴, X⁵ and X⁶ may be present or absent such that the total number of nucleotides in the G-rich region is from 6 to 9.

16. The method according to claim 9 wherein the (R⁵-R¹-R²-R³-R⁴) tract is devoid of adenosine nucleotides and the G-rich region has the formula 6.2:

| | |
|---|---|
| 5′ [(G-G-G-G-G)-X³-X⁴-X⁵-X⁶] 3′ | Formula 6.2 |

wherein X³ represents A or C, and
X⁴, X⁵ and X⁶ have the previously defined meanings, and may be present or absent such that the total number of nucleotides in the G-rich region is from 7 to 9.

17. The method according to claim 10 wherein X¹ in any one of Formulae 5.1.1, 5.1.2, 5.1.3, 5.1.4, 5.1.5 represents T or C.

18. The method according to claim 17 wherein the 5′ G-rich region has the sequence:
5′ TGAGGG 3′

19. The method according to claim 17 wherein the 5′ G-rich region has the sequence:
5′ CGGGAG 3′

20. The method according to claim 17 wherein the 5′ G-rich region has the sequence:
5′ TAGGGG 3′

21. The method according to claim 11 or 12 wherein X¹ in Formulae 5.1.1, 5.1.2, 5.1.3, 5.1.4, 5.1.5 represents A or G.

22. The method according to claim 21 wherein the 5′ G-rich region has the sequence:
5′ GAGGGG 3′

23. The method according to claim 15 wherein X³ in any one of Formulae 6.1.1, 6.1.2, 6.1.3, 6.1.4, 6.1.5 represents A or C and the G-rich region has 7, 8 or 9 nucleotides.

24. The method according to claim 23 wherein the 5′ G-rich region has the sequence:
5′ AGGGGCAG 3′

25. The method according to claim 15 wherein X³ in Formulae 6.1.1, 6.1.2, 6.1.3, 6.1.4, 6.1.5 represents G and the G-rich region has 6 nucleotides.

26. The method according to claim 25 wherein the 5′ G-rich region has the sequence:
5′ GGGAGG 3′

27. The method according to claim 25 wherein the 5′ G-rich region has the sequence:
5′ AGGGAG 3′

28. The method according to claim 25 wherein the 5′ G-rich region has the sequence:
5′ AGGGGG 3′

29. The method according to any one of claims 1, wherein the oligonucleotide comprises a 3′ tail region which contains only purine nucleotides.

30. The method according to claim 1, wherein the oligonucleotide comprises a 3′ tail region containing purine and pyrimidine nucleotides.

31. The method according to claim 30 wherein the 3′ tail region of the oligonucleotide is generated randomly from an equimolar mix of A, C, T and G nucleotides.

32. The method according to of claim 1 wherein the oligonucleotide has a length of 26 to 45 nucleotides.

33. The method according to of claim 1 wherein the oligonucleotide has a length of 30 to 40 nucleotides.

34. The method according to claims 1 wherein the tail region of the oligonucleotide contains two sequences capable of together forming a hairpin structure within the tail.

35. The method according to of claim 1 wherein the oligonucleotide is devoid of functional DNAzyme catalytic motifs, such as 5′-GGCTAGCTACAACGA-3′ (SEQ ID NO: 86).

36. The method according to claim 18, wherein the oligonucleotide is chosen from the group consisting of:
(SEQ ID NO: 5)

| | |
|---|---|
| Oligo 1 | TGAGGGGCAGGCTAGCTACAACGACGTCGTGA(3′-3′C) |

(SEQ ID NO: 14)

| | |
|---|---|
| Oligo 2 | TGAGGGGCAAGCAACATCGATCGGCGTCGTGA(3′-3′C) |

(SEQ ID NO: 6)

| | |
|---|---|
| Oligo 3 | TGAGGGGCAGGCTAGCTACAACGACGTCGC |
| | GG(3′-3′G) |

(SEQ ID NO: 2)

| | |
|---|---|
| Oligo 14 | (5′-5′T)GAGGGGCAGGCTAGCTACAACGACGTCGTGAC |

(SEQ ID NO: 3)

| | |
|---|---|
| Oligo 15 | (5′-5′T)GAGGGGCAGGCTAGCTACAACGACGTCGTGA |
| | (3′-3′C) |

(SEQ ID NO: 9)

| | |
|---|---|
| Oligo 16 | TGAGGGGCAGGCTAGCTACAACGACGTCGTGAC |
| | (with 9 + 9 phosphorothioate linkages) |

(SEQ ID NO: 10)

| | |
|---|---|
| Oligo 17 | TGAGGGGCAGGCTAGCTACAACGACGTCGTGAC |
| | (with 7 + 7 phosphorothioate linkages) |

(SEQ ID NO: 11)

| | |
|---|---|
| Oligo 18 | TGAGGGGCAGGCTAGCTACAACGACGTCGTGAC |
| | (with 5 + 5 phosphorothioate linkages) |

(SEQ ID NO: 13)

| | |
|---|---|
| Oligo 25 | TGAGGGGCAGGCTAGCTACAACGACGTCGTGAC |

(SEQ ID NO: 16)

| | |
|---|---|
| Oligo 66 | TGAGGGGCAGN₂₅ |

(SEQ ID NO: 17)

| | |
|---|---|
| Oligo 67 | TGAGGGGCN₂₇ |

(SEQ ID NO: 18)

| | |
|---|---|
| Oligo 68 | TGAGGGN₂₉ |

where each N independently represents G, T, C or A, and may be the same or different, and (3′-3′) and (5′-5′) signifies an inverted 3′ or 5′ linkage respectively.

37. The method according to claim 19, wherein the oligonucleotide is chosen from the group consisting of:
(SEQ ID NO: 20)

| | |
|---|---|
| Oligo 4 | CGGGAGGAAGGCTAGCTACAACGAGAGGCGTTG(3′-3′T) |

(SEQ ID NO: 26)

| | |
|---|---|
| Oligo 9 | CₘGGGAGGAAGGCTAGCTACAACₘGAGAGGCₘGTTG |
| | (3′-3′T) |

(SEQ ID NO: 23)

| | |
|---|---|
| Oligo 10 | CGGGAGGAAGGCTAGCTACAAGCAGAGGGCTTG(3′-3′T) |

(SEQ ID NO: 28)

| | |
|---|---|
| Oligo 11 | CGGGAGGAAGGCTAGCTACAACGAGAGGCGTTG-X |

(SEQ ID NO: 39)

| | |
|---|---|
| Oligo 12 | CGGGAGGAAG(N₂₀) |

(SEQ ID NO: 40)

| | |
|---|---|
| Oligo 13 | CGGGAGGAAG(N₂₅) |

(SEQ ID NO: 25)

| | |
|---|---|
| Oligo 19 | CGGGAGGAAGGCTAGCTACAACGAGAGGCGTTG |

(SEQ ID NO: 27)

| | |
|---|---|
| Oligo 20 | CGGGAGGAAGGCTAGCTACAACGAGAGGCGTUG(3′-3′T) |
| | (with 2 × 2′-Omethyl at both ends) |

(SEQ ID NO: 21)

| | |
|---|---|
| Oligo 27 | CGGGAGGAAGGCTAGCTACAACAAGAGGCGTTG(3′-3′T) |

(SEQ ID NO: 22)

| | |
|---|---|
| Oligo 28 | CGGGAGGAAAGCAACATCGATCGG(3′-3′T) |

(SEQ ID NO: 24)

| | |
|---|---|
| Oligo 29 | CGGGAGGAAGGCTAGCACACAGAGGGTCATGGT(3′-3′T) |

(SEQ ID NO: 41)

| | |
|---|---|
| Oligo 30 | CGGGAGGAAG(N₂₃)[3′-3′T] |

(SEQ ID NO: 29)

| | |
|---|---|
| Oligo 31 | (5′P)CGGGAGGAAGGCTAGCTACAACGAGAGGCGTTG |

(SEQ ID NO: 45)

| | |
|---|---|
| Oligo 43 | CGGGAGGAAG(N₁₅) |

(SEQ ID NO: 47)

| | |
|---|---|
| Oligo 63 | CGGGAGGA(N₂₇) |

(SEQ ID NO: 48)

| | |
|---|---|
| Oligo 64 | CGGGAG(N₂₉) |

(SEQ ID NO: 42)

| | |
|---|---|
| Oligo 70 | CGGGAGGAAG(TAG)₈ |

(SEQ ID NO: 46)

| | |
|---|---|
| Oligo 83 | CGGGAGGAAGGCTAGCTACAACGAGAGGCGTTG-B |

where each N independently represents G, T, C or A, and may be the same or different, X represents cholesteryl-TEG, (5′P) represents a 5′ phosphorylation, Cₘ represents a methylated cytosine, B represents biotin, and (3′-3′) and (5′-5′) signifies an inverted 3′ or 5′ linkage respectively.

38. The method according to claim 22, wherein the oligonucleotide is chosen from the group consisting of:
(SEQ ID NO: 65)

| | |
|---|---|
| Oligo 101 | GAGGGGGAAGGCTAGCTACAACGAAGTTCGTCC |

(SEQ ID NO: 4)

| | |
|---|---|
| Oligo 24 | GAGGGGCAGGCTAGCTACAACGACGTCGTGA |

39. The method according to claim 26, wherein the oligonucleotide is chosen from the group consisting of:
(SEQ ID NO: 30)

| | |
|---|---|
| Oligo 8 | GGGAGGAAGGCTAGCTACAACGAGAGGCGTT(3′-3′T) |

(SEQ ID NO: 54)

| | |
|---|---|
| Oligo 72 | GGGAGGAAAGN₂₅ |

(SEQ ID NO: 55)

| | |
|---|---|
| Oligo 73 | GGGAGGAAAGN₂₀ |

(SEQ ID NO: 56)

| | |
|---|---|
| Oligo 74 | GGGAGGAAAGN₁₅ |

where each N independently represents G, T, C or A, and may be the same or different, and (3′-3′) signifies an inverted 3″ linkage.

40. The method according to claim 27, wherein the oligonucleotide is chosen from the group consisting of:
(SEQ ID NO: 60)

| | |
|---|---|
| Oligo 80 | AGGGAGGGAGGAAGGGAGGGAGGGAGGG |

(SEQ ID NO: 61)

| | |
|---|---|
| Oligo 81 | AGGGAGGGAGGAAGGGAGGGAGGGAGGGAGGG |

41. The method according to claim 24, wherein the oligonucleotide is:
(SEQ ID NO: 1)

| | |
|---|---|
| Oligo 23 | AGGGGCAGGCTAGCTACAACGACGTCGTG |

42. The method according to claim 6, wherein the oligonucleotide is:
(SEQ ID NO: 67)

| | |
|---|---|
| Oligo 26 | GGGGCAGGAAGCAACATCGATCGGGACTTTTGA |

43. The method according to claim 4, wherein the oligonucleotide is:
(SEQ ID NO: 66)

| | |
|---|---|
| Oligo 100 | GCGGGGACAGGCTAGCTACAACGACAGCTGCAT |

44. The method according to claim 1 wherein the oligonucleotide consists of DNA.

45. The method according to claim 1 wherein the oligonucleotide comprises a mixture of DNA and RNA.

46. The method according to claim 1 wherein the oligonucleotide comprises a mixture of DNA and DNA analogues.

47. The method according to claim 1 wherein the oligonucleotide contains chemically modified nucleotides.

48. The method according to claim 1 wherein the oligonucleotide is single stranded.

49. The method according to claim 1 wherein the oligonucleotide induces cell death having at least one characteristic of programmed cell death in at least one of the following cell types: vascular endothelial cells, vascular smooth muscle cells, fibroblasts, neoplastic cells, retinal epithelium.

50. The method according to claim 49 wherein the induced cell death having at least one characteristic of programmed cell death, is accompanied by inhibition of cell proliferation.

51. The method according to claim 1 wherein the disorder involving abnormal cell proliferation and/or migration is an angiogenesis related disorder, such as psoriasis, age-related macular degeneration (AMD), diabetic retinopathy, cancer, arthritis.

52. The method according to claim 1 wherein the disorder involving abnormal cell proliferation and/or migration is a disease associated with smooth muscle proliferation such as post-angioplasty restenosis, atherosclerosis, pulmonary hypertension, asthma.

53. The method according to claim 1 wherein the disorder involving abnormal cell proliferation and/or migration is an inflammatory disorder, such as ocular inflammation, uveitis, retinitis.

54. The method according to claim 1 wherein the disorder involving abnormal cell proliferation and/or migration is corneal neovascularisation.

55. The method according to claim 1 wherein the disorder involving abnormal cell proliferation and/or migration is tumour growth or metastasis.

56. A method of treating or preventing a disorder involving aberrant cell proliferation or migration, comprising administering to a patient an oligonucleotide which induces, in non-quiescent eukaryotic cells, cell death
wherein said oligonucleotide is to be administered to a subject in an amount such that it induces cell death having at least one characteristic of programmed cell death in said cells in said subject,
wherein said oligonucleotide has a length of 20 to 50 nucleotides and consists of
i) a 5′ G-rich region having 6 to 9 nucleotides, and
ii) a 3′ tail region,
wherein the 5′ G-rich region has the formula 7:
Formula 7
5′ [R⁶-R⁵-(R¹-R²-R³-R⁴)-R⁷-R⁸-R⁹-R¹⁰-R¹¹] 3′
in which
each R represents a purine nucleotide,
(R¹-R²-R³-R⁴) represents a tract of four consecutive purine nucleotides,
each of R⁵, R⁶, R⁷, R⁸, R⁹, R¹⁰ and R¹¹ independently represents a purine nucleotide which may be present or absent, such that the total number of nucleotides in the G-rich region is from 6 to 9,
provided that:
at least 50% of the nucleotides in the G-rich region are guanosine nucleotides,
the portion of the G-rich region represented by R⁵-(R¹-R²-R³-R⁴) contains a triple guanosine motif (G-G-G)
the G-rich region is not composed exclusively of guanosine nucleotides,
the nucleotide defining the 3′ extremity of the G-rich region is a guanosine nucleotide,
and the 3′ tail region consists of purine nucleotides.

57. The method according to claim 56, wherein the G-rich region of the oligonucleotide is chosen from the group consisting of:

| | |
|---|---|
| 5′ [R⁶-(AGGGG)] 3′ | Formula 7.1 |
| 5′ [R⁶-(GAGGG)] 3′ | Formula 7.2 |
| 5′ [R⁶-(GGGAG)] 3′ | Formula 7.3 |
| 5′ [R⁶-(GGGGA)-R⁷-R⁸-R⁹] 3′ | Formula 7.4 |
| 5′ [R⁶-(AGGGA)-R⁷-R⁸-R⁹] 3′ | Formula 7.5 |
| 5′ [(AGGGG)-R⁷-R⁸-R⁹-R¹⁰] 3′ | Formula 7.6 |
| 5′ [(GAGGG)-R⁷-R⁸-R⁹-R¹⁰] 3′ | Formula 7.7 |
| 5′ [(GGGAG)-R⁷-R⁸-R⁹-R¹⁰] 3′ | Formula 7.8 |
| 5′ [(GGGGA)-R⁷-R⁸-R⁹-R¹⁰] 3′ | Formula 7.9 |
| 5′ [(AGGGA)-R⁷-R⁸-R⁹-R¹⁰] 3′ | Formula 7.10 |

wherein each of R⁶ R⁷ R⁸, R⁹, R¹⁰ independently represent a purine nucleotide, and may be present or absent such that the total number of nucleotides in the G-rich region is from 6 to 9.

58. The method according to claim 56, wherein the oligonucleotide has a length from 20 to 24 nucleotides.

59. The method according to claims 56, wherein the G-rich region does not contain two consecutive adenosine nucleotides.

60. The method according to claim 57, wherein the oligonucleotide is chosen from the group consisting of

| | | |
|---|---|---|
| Oligo 79 | AGGGAGGGAGGAAGGGAGGGAGGG | (SEQ ID NO: 59) |
| Oligo 82 | (AGGG)₆ | (SEQ ID NO: 62) |
| Oligo 78 | AGGGAGGGAGGAAGGGAGGG | (SEQ ID NO: 63) |

61. An Oligonucleotide capable of inducing, in non-quiescent eukaryotic cells, cell death having at least one characteristic of programmed cell death, said oligonucleotide having a length of 25 to 50 nucleotides and consisting of
i) a 5′ G-rich region having from 6 to 9 nucleotides, and
ii) a 3′ tail region,
wherein the 5′ G-rich region has the formula 1a:
Formula 1a
5′ [X¹-X²-(R¹-R²-R³-R⁴)-X³-X⁴-X⁵-X⁶-X⁷] 3′
in which
(R¹-R²-R³-R⁴) represents a tract of four consecutive purine nucleotides, each R representing a purine nucleotide,
each of X¹, X², X³, X⁴, X⁵, X⁶ and X⁷ independently represents a nucleotide which may be present or absent, such that the total number of nucleotides in the G-rich region is from 6 to 9,
each of X¹, X² X³, X⁴, X⁵, X⁶ and X⁷ independently represents a purine or pyrimidine nucleotide,
provided that:
at least 50% of the nucleotides in the G-rich region are guanosine nucleotides,
the portion of the G-rich region represented by X²-(R¹-R²-R³-R⁴) contains a triple guanosine motif (G-G-G),
the portion of the G-rich region represented by X³-X⁴-X⁵-X⁶-X⁷ does not contain a thymidine nucleotide downstream of a guanosine nucleotide,
the nucleotide defining the 3′ extremity of the G-rich region is a guanosine nucleotide,
the total number of pyrimidine nucleotides in the G-rich region does not exceed 2, and these pyrimidine nucleotides are not consecutive to each other,
if the first 4 nucleotides at the 5′ end of the G-rich region are 4 consecutive guanosine nucleotides, the fifth nucleotide of the G-rich region is a cytosine nucleotide,
and the 3′ tail region is any nucleotide sequence,
provided the oligonucleotide does not contain the sequence 5′-GGCTANCTACAACGA-3′ (SEQ ID NO: 88), or its inverse sequence 5′-AGCAACATCNATCGG-3′ (SEQ ID NO: 89) wherein N represents a guanosine or cytosine nucleotide.

62. An Oligonucleotide according to claim 61 wherein X¹ is present or absent, X² is present and is a pyrimidine nucleotide (Py), and the 5′ G-rich region has the Formula 2:

| | |
|---|---|
| 5′ [X¹-Py-(R¹-R²-R³-R⁴)-X³-X⁴-X⁵-X⁶] 3′ | Formula 2 |

wherein R¹, R², R³, R⁴, X¹, X³, X⁴, X⁵ and X⁶ have the previously defined meanings, and X³ , X⁴ , X⁵ and X⁶ may be present or absent such that the total number of nucleotides in the G-rich region is from 6 to 9.

63. An Oligonucleotide according to claim 62 wherein Py in Formula 2 is a cytosine nucleotide.

64. An Oligonucleotide according to claim 63 wherein the G-rich region has the sequence:
5′ GCGGGG 3′

65. An Oligonucleotide according to claim 61 wherein X¹ and X² are absent, X³ represents a cytosine nucleotide and the 5′ G-rich region has the Formula 3:

| | |
|---|---|
| 5′ [(R¹-R²-R³-R⁴)-C-X⁴-X⁵-X⁶-X⁷] 3′ | Formula 3 |

wherein R, X⁴, X⁵, X⁶ and X⁷ have the previously defined meanings, and X³, X⁴ X⁵, X⁶ and X⁷ may be present or absent such that the total number of nucleotides in the G-rich region is from 6 to 9.

66. An Oligonucleotide according to claim 61 wherein the G-rich region comprises the sequence
5′ GGGGCAG 3′.

67. An Oligonucleotide according to claim 61 wherein X¹ is present, X² is present and is a purine nucleotide (R⁵), and the G-rich region has the formula 5.1a:

| | |
|---|---|
| 5′ [X¹-(R⁵-R¹-R²-R³-R⁴)-X³-X⁴-X⁵] 3′ | Formula 5.1a |

wherein
(R⁵-R¹-R²-R³-R⁴) represents a tract of five consecutive purine nucleotides containing a triple guanosine (G-G-G) motif,
at least one of R⁵ and R¹ represents A,
X¹ represents a purine or pyrimidine nucleotide, and
X³, X⁴, and X⁵ have the previously defined meanings, and may be present or absent such that the total number of nucleotides in the G-rich region is from 6 to 9.

68. An Oligonucleotide according to claim 61 wherein X¹ is present, X² is present and is a purine nucleotide (R⁵), and the G-rich region has the formula 5.1b:

| | |
|---|---|
| 5′ [X¹-(R⁵-R¹-R²-R³-R⁴)-X³-X⁴-X⁵] 3′ | Formula 5.1b |

wherein
(R⁵-R¹-R²-R³-R⁴) represents a tract of five consecutive purine nucleotides containing a triple guanosine (G-G-G) motif,
at least one of R³ and R⁴ represents A,
X¹ represents A, C or T and
X³, X⁴, and X⁵ have the previously defined meanings, and may be present or absent such that the total number of nucleotides in the G-rich region is from 6 to 9.

69. An Oligonucleotide according to claim 61 wherein X¹ is present, X² is present and is a guanosine nucleotide (G), the purine tract is devoid of adenosine nucleotides, and the G-rich region has the formula 5.2:

| | |
|---|---|
| 5′ [X¹-(G-G-G-G-G)] 3′ | Formula 5.2 |

wherein
X¹ represents A, C or T.

70. An Oligonucleotide according to claim 61 wherein X¹ is absent, X2 is present and is a purine nucleotide (R⁵), and the G-rich region has the formula 6.1:

| | |
|---|---|
| 5′ [(R⁵-R¹-R²-R³-R⁴)-X³-X⁴-X⁵-X⁶] 3′ | Formula 6.1 |

wherein
(R⁵-R¹-R²-R³-R⁴) represents a tract of five consecutive purine nucleotides containing a triple guanosine (G-G-G) motif,
at least one of R⁵, R¹, R³ and R⁴ represents A,
X³ , X⁴, X⁵ and X⁶ have the previously defined meanings, and X⁴, X⁵ and X⁶ may be present or absent such that the total number of nucleotides in the G-rich region is from 6 to 9.

71. An Oligonucleotide according to claim 67 wherein the 5′ G-rich region is chosen from the group consisting of:

| | |
|---|---|
| 5′ [X¹-(AGGGG)] 3′ | Formula (5.1.1) |
| 5′ [X¹-(GAGGG)] 3′ | Formula (5.1.2) |
| 5′ [X¹-(AGGGA)-X³-X⁴-X⁵] 3′ | Formula (5.1.5) |

wherein A represents an adenosine nucleotide and G represents a guanosine nucleotide,
X¹ represents a purine or pyrimidinde nucleotide,
X³, X⁴ and X⁵ have the previously defined meanings, and X⁴ and X⁵ may be present or absent such that the total number of nucleotides in the G-rich region is 7, 8 or 9.

72. An Oligonucleotide according to claim 68 wherein the 5′ G-rich region is chosen from the group consisting of:

| | |
|---|---|
| 5′ [X¹-(GGGAG)] 3′ | Formula (5.1.3b) |
| 5′ [X¹-(GGGGA)-X³-X⁴-X⁵] 3′ | Formula (5.1.4b) |

wherein A represents an adenosine nucleotide and G represents a guanosine nucleotide,
X¹ represents A, C or T,
X³, X⁴, and X⁵ have the previously defined meanings, and X⁴ and X⁵ may be present or absent such that the total number of nucleotides in the G-rich region is 7, 8 or 9.

73. An Oligonucleotide according to claim 70 wherein the 5′ G-rich region is chosen from the group consisting of:

| | |
|---|---|
| 5′ [(AGGGG)-X³-X⁴-X⁵-X⁶] 3′ | Formula (6.1.1) |
| 5′ [(GAGGG)-X³-X⁴-X⁵-X⁶] 3′ | Formula (6.1.2) |
| 5′ [(GGGAG)-X³-X⁴-X⁵-X⁶] 3′ | Formula (6.1.3) |
| 5′ [(AGGGA)-X³-X⁴-X⁵-X⁶] 3′ | Formula (6.1.5) |

wherein A represents an adenosine nucleotide, and G represents a guanosine nucleotide,
and X³, X⁴, X⁵ and X⁶ have the previously defined meanings, and may be present or absent such that the total number of nucleotides in the G-rich region is from 6 to 9.

74. An Oligonucleotide according to claim 71 or 72 wherein X¹ in any one of Formulae (5.1.1), (5.1.2), (5.1.5), (5.1.3b), (5.1.4b) represents T or C.

75. An Oligonucleotide according to claim 74 wherein the 5′ G-rich region has the sequence:
5′ TGAGGG 3′

76. An Oligonucleotide according to claim 74 wherein the 5′ G-rich region has the sequence:
5′ CGGGAG 3′

77. An Oligonucleotide according to claim 74 wherein the 5′ G-rich region has the sequence:
5′ TAGGGG 3′

78. An Oligonucleotide according to claim 71 wherein X¹ in any one of Formulae (5.1.1), (5.1.2), (5.1.5) represents A or G.

79. An Oligonucleotide according to claim 78 wherein the 5′ G-rich region has the sequence:
5′ GAGGGG 3′

80. An Oligonucleotide according to claim 73 wherein X³ in any one of Formulae (6.1.1), (6.1.2), (6.1.3), (6.1.5), represents A or C and the G-rich region has 7, 8 or 9 nucleotides.

81. An Oligonucleotide according to claim 80 wherein the 5′ G-rich region has the sequence:
5′ AGGGGCAG 3′

82. An Oligonucleotide according to claim 73 wherein X³ in any one of Formulae (6.1.1), (6.1.2), (6.1.3), (6.1.5) represents G and the G-rich region has 6 nucleotides.

83. An Oligonucleotide according to claim 82 wherein the 5′ G-rich region has the sequence:
5′ GGGAGG 3′

84. An Oligonucleotide according to claim 82 wherein the 5′ G-rich region has the sequence:
5′ AGGGAG 3′

85. An Oligonucleotide according to claim 82 wherein the 5′ G-rich region has the sequence:
5′ GAGGGG 3′

86. An Oligonucleotide according to claim 61, wherein the oligonucleotide further comprises a 3′ tail region which contains only purine nucleotides.

87. An Oligonucleotide according to claim 61, wherein the oligonucleotide further comprises a 3′ tail region which contains each of the nucleotides A, C, T and G.

88. An Oligonucleotide according to claim 87 wherein the 3′ tail region of the oligonucleotide is generated randomly from an equimolar mix of A, C, T and G nucleotides.

89. An Oligonucleotide according to claim 61, wherein the oligonucleotide has a length of 26 to 45 nucleotides.

90. An Oligonucleotide according to claim 61, wherein the oligonucleotide has a length of 30 to 40 nucleotides.

91. An Oligonucleotide according to claim 61, wherein the tail region of the oligonucleotide contains two sequences capable of together forming a hairpin structure within the tail.

92. An Oligonucleotide according to claim 61, wherein the tail region of the oligonucleotide is devoid of sequences capable of forming a hairpin structure with sequences within the G-rich region.

93. An Oligonucleotide according to claim 75, wherein the oligonucleotide is chosen from the group consisting of:

| | | |
|---|---|---|
| Oligo 66 | TGAGGGGCAGN₂₅ | (SEQ ID NO: 16) |
| Oligo 67 | TGAGGGGCN₂₇ | (SEQ ID NO: 17) |
| Oligo 68 | TGAGGGN₂₉ | (SEQ ID NO: 18) |

where each N independently represents G, T, C or A, and may be the same or different.

94. An Oligonucleotide according to claim 76, wherein the oligonucleotide is chosen from the group consisting of:
(SEQ ID NO: 23)

| | |
|---|---|
| Oligo 10 | CGGGAGGAAGGCTAGCTACAAGCAGAGGGCTTG(3′-3′T) |

(SEQ ID NO: 21)

| | |
|---|---|
| Oligo 27 | CGGGAGGAAGGCTAGCTACAACAAGAGGCGTTG(3′-3′T) |

(SEQ ID NO: 24)

| | |
|---|---|
| Oligo 29 | CGGGAGGAAGGCTAGCACACAGAGGGTCATGGT(3′-3′T) |

where each N independently represents G, T, C or A, and may be the same or different, and (3′-3′) signifies an inverted 3′ linkage .

95. An Oligonucleotide according to claim 76, wherein the oligonucleotide is chosen from the group consisting of:

| | | |
|---|---|---|
| Oligo 12 | CGGGAGGAAG(N₂₀) | (SEQ ID NO: 39) |
| Oligo 13 | CGGGAGGAAG(N₂₅) | (SEQ ID NO: 40) |
| Oligo 30 | CGGGAGGAAG(N₂₃)[3′-3′T] | (SEQ ID NO: 41) |
| Oligo 43 | CGGGAGGAAG(N₁₅) | (SEQ ID NO: 45) |
| Oligo 63 | CGGGAGGAN₂₇ | (SEQ ID NO: 47) |
| Oligo 64 | CGGGAGN₂₉ | (SEQ ID NO: 48) |
| Oligo 70 | CGGGAGGAAG(TAG)₈ | (SEQ ID NO: 42) |

96. An Oligonucleotide according to claim 83, wherein the oligonucleotide is:

| | | |
|---|---|---|
| Oligo 72 | GGGAGGAAAGN₂₅ | (SEQ ID NO: 54) |

where N represents G, T, C or A.

97. An Oligonucleotide according to claim 83, wherein the oligonucleotide is:

| | | |
|---|---|---|
| Oligo 73 | GGGAGGAAAGN₂₀ | (SEQ ID NO: 55) |

where N represents G, T, C or A.

98. An Oligonucleotide according to claim 83, wherein the oligonucleotide is:

| | | |
|---|---|---|
| Oligo 74 | GGGAGGAAAGN₁₅ | (SEQ ID NO: 56) |

where N represents G, T, C or A.

99. An Oligonucleotide according to claim 84, wherein the oligonucleotide is chosen from the group consisting of:
(SEQ ID NO: 60)

| | |
|---|---|
| Oligo 80 | AGGGAGGGAGGAAGGGAGGGAGGGAGGG |

(SEQ ID NO: 61)

| | |
|---|---|
| Oligo 81 | AGGGAGGGAGGAAGGGAGGGAGGGAGGGAGGG |

100. An Oligonucleotide according to claim 61, wherein the oligonucleotide consists of DNA.

101. An Oligonucleotide according to claim 61, wherein the oligonucleotide comprises a mixture of DNA and RNA

102. An Oligonucleotide according to claim 61, wherein the oligonucleotide comprises a mixture of DNA and DNA analogues.

103. An Oligonucleotide according to claim 61, wherein the oligonucleotide contains chemically modified nucleotides.

104. An Oligonucleotide according to claim 103 which is modified in that it comprises
at least one nucleotide which is modified at the 2′-OH position,
or at least one methylated cytosine,
or is substituted at the 3′ terminal by groups such as cholesterol, biotin, dyes, markers;
or has a partially modified phosphodiester backbone.

105. An Oligonucleotide according to claim 61, wherein the oligonucleotide is single stranded.

106. An Oligonucleotide capable of inducing, in non-quiescent eukaryotic cells, cell death having at least one characteristic of programmed cell death,
said oligonucleotide consisting of
i) a 5′ G-rich region having 6 nucleotides, and
ii) a 3′ tail region,
wherein the oligonucleotide is a variant of the sequence
(SEQ ID NO: 13)

| | |
|---|---|
| Oligo 25 | TGAGGGGCAGGCTAGCTACAACGACGTCGTGAC, |

said variant being obtainable by carrying out at least one of the following modifications on the said sequence:
a 5′ terminal inversion,
a partial modification of the phosphodiester backbone wherein at least five phosphodiester linkages at both 5′ and 3′ extremities are modified,
a truncation of one or two nucleotides at the 5′ and/or 3′ extremities,
substitution of the nucleotides TGAC at the 3′ extremity by CGG(3′-3′G).

107. An Oligonucleotide capable of inducing, in non-quiescent eukaryotic cells, cell death having at least one characteristic of programmed cell death,
said oligonucleotide consisting of
iii) a 5′ G-rich region having 6 nucleotides, and
iv) a 3′ tail region,
wherein the oligonucleotide has the sequence
(SEQ ID NO: 25)

| | |
|---|---|
| Oligo 19 | CGGGAGGAAGGCTAGCTACAACGAGAGGCGTTG |

and is modified in that it comprises
at least one nucleotide which is modified at the 2′-OH position,
or at least one methylated cytosine,
or is substituted at the 3′ terminal by groups such as cholesterol, biotin, dyes, markers;
or has a partially modified phosphodiester backbone.

108. An Oligonucleotide capable of inducing, in non-quiescent eukaryotic cells, cell death having at least one characteristic of programmed cell death,
wherein said oligonucleotide has a length of 21 to 50 nucleotides and consists of
i) a 5′ G-rich region having 6 to 9 nucleotides, and
ii) a 3′ tail region,
wherein the 5′ G-rich region has the formula 7:
Formula 7
5′ [R⁶-R⁵-(R¹-R²-R³-R⁴)-R⁷-R⁸-R⁹-R¹⁰-R¹¹] 3′
in which
each R represents a purine nucleotide,
(R¹-R²-R³-R⁴) represents a tract of four consecutive purine nucleotides,
each of R⁵, R⁶, R⁷, R⁸, R⁹, R¹⁰ and R¹¹ independently represents a purine nucleotide which may be present or absent, such that the total number of nucleotides in the G-rich region is from 6 to 9,
provided that:
at least 50% of the nucleotides in the G-rich region are guanosine nucleotides,
the portion of the G-rich region represented by R⁵-(R¹-R²-R³-R⁴) contains a triple guanosine motif (G-G-G)
the G-rich region is not composed exclusively of guanosine nucleotides,
the nucleotide defining the 3′ extremity of the G-rich region is a guanosine nucleotide,
and the 3′ tail region consists of any sequence of purine nucleotides.

109. An Oligonucleotide according to claim 108 wherein the G-rich region of the oligonucleotide is chosen from the group consisting of:

| | |
|---|---|
| 5′ [R⁶-(AGGGG)] 3′ | Formula 7.1 |
| 5′ [R⁶-(GAGGG)] 3′ | Formula 7.2 |
| 5′ [R⁶-(GGGAG)] 3′ | Formula 7.3 |
| 5′ [R⁶-(GGGGA)-R⁷-R⁸-R⁹] 3′ | Formula 7.4 |
| 5′ [R⁶-(AGGGA)-R⁷-R⁸-R⁹] 3′ | Formula 7.5 |
| 5′ [(AGGGG)-R⁷-R⁸-R⁹-R¹⁰] 3′ | Formula 7.6 |
| 5′ [(GAGGG)-R⁷-R⁸-R⁹-R¹⁰] 3′ | Formula 7.7 |
| 5′ [(GGGAG)-R⁷-R⁸-R⁹-R¹⁰] 3′ | Formula 7.8 |
| 5′ [(GGGGA)-R⁷-R⁸-R⁹-R¹⁰] 3′ | Formula 7.9 |
| 5′ [(AGGGA)-R⁷-R⁸-R⁹-R¹⁰] 3′ | Formula 7.10 |

wherein each of R⁶, R⁷, R⁸, R⁹, R¹⁰ independently represent a purine nucleotide, and may be present or absent such that the total number of nucleotides in the G-rich region is from 6 to 9.

110. An Oligonucleotide according to claim 109, wherein the G-rich region has the sequence:
5′ AGGGAG 3′

111. An Oligonucleotide according to claim 110, wherein the oligonucleotide is chosen from the group consisting of:
(SEQ ID NO: 59)

| | |
|---|---|
| Oligo 79 | AGGGAGGGAGGAAGGGAGGGAGGG |

(SEQ ID NO: 60)

| | |
|---|---|
| Oligo 80 | AGGGAGGGAGGAAGGGAGGGAGGGAGGG |

(SEQ ID NO: 61)

| | |
|---|---|
| Oligo 81 | AGGGAGGGAGGAAGGGAGGGAGGGAGGGAGGG |

(SEQ ID NO: 62)

| | |
|---|---|
| Oligo 82 | (AGGG)₆ |

112. A pharmaceutical composition comprising as active principle at least one oligonucleotide according to claim 61, in association with a pharmaceutically acceptable carrier.

113. A pharmaceutical composition according to claim 112, comprising at least two oligonucleotides as active principle.

114. A pharmaceutical composition containing an oligonucleotide according to claim 61, in association with an additional therapeutic agent as a combined preparation for simultaneous, separate or sequential use in therapy.

115. A pharmaceutical composition containing an oligonucleotide according to claim 61, in association with an additional therapeutic agent as a combined preparation for simultaneous, separate or sequential use in therapy of disorders involving abnormal cell proliferation or migration.

116. A method for inducing cell death in a population of non-quiescent eukaryotic cells, said method comprising introducing at least one oligonucleotide according to claim 61, into cells of said population, in an amount sufficient to induce cell death having at least one characteristic of programmed cell death, in at least a portion of the population of cells containing said oligonucleotide.

117. The method according to claim 116, wherein the cell population is a population within a higher eukaryotic organism and the method is carried out in vivo.

118. The method according to claim 117, wherein the higher eukaryotic organism is a mammal.

119. The method according to claim 117 wherein the non-quiescent cell population is a population of vascular endothelial cells, vascular smooth muscle cells, fibroblasts, neoplastic cells, or retinal epithelium.

120. The method according to claim 116, wherein the cell population is a population in cell culture and the method is carried out in vitro.

121. Isolated eukaryotic cell containing an oligonucleotide according to claim 61.
